SEQUENCE LISTING (179 sequences)

SEQ ID NO: 1 - DNA, 20 bp, synthetic construct
  misc_feature 1..20: note = B2M gRNA-1
  source 1..20: mol_type = other DNA
actcacgctggatagcctcc

SEQ ID NO: 2 - DNA, 20 bp, synthetic construct
  misc_feature 1..20: note = B2M gRNA-2
  source 1..20: mol_type = other DNA
agtcacatggttcacacggc

SEQ ID NO: 3 - DNA, 20 bp, synthetic construct
  misc_feature 1..20: note = B2M gRNA-3
  source 1..20: mol_type = other DNA
ggccgagatgtctcgctccg

SEQ ID NO: 4 - DNA, 20 bp, synthetic construct
  misc_feature 1..20: note = B2M gRNA-4
  source 1..20: mol_type = other DNA
gcatactcatctttttcagt

SEQ ID NO: 5 - DNA, 20 bp, synthetic construct
  misc_feature 1..20: note = B2M gRNA-5
  source 1..20: mol_type = other DNA
tcacgtcatccagcagagaa

SEQ ID NO: 6 - DNA, 20 bp, synthetic construct
  misc_feature 1..20: note = B2M gRNA-6
  source 1..20: mol_type = other DNA
gtcttttcccgatattcctc

SEQ ID NO: 7 - DNA, 20 bp, synthetic construct
  misc_feature 1..20: note = B2M gRNA-7
  source 1..20: mol_type = other DNA
acttgtctttcagcaaggac

SEQ ID NO: 8 - DNA, 20 bp, synthetic construct
  misc_feature 1..20: note = B2M gRNA-8
  source 1..20: mol_type = other DNA
agggtaggagagactcacgc

SEQ ID NO: 9 - DNA, 20 bp, synthetic construct
  misc_feature 1..20: note = B2M gRNA-9
  source 1..20: mol_type = other DNA
ttcagacttgtctttcagca

SEQ ID NO: 10 - DNA, 20 bp, synthetic construct
  misc_feature 1..20: note = B2M gRNA-10
  source 1..20: mol_type = other DNA
ttaccccacttaactatctt

SEQ ID NO: 11 - DNA, 20 bp, synthetic construct
  misc_feature 1..20: note = B2M gRNA-11
  source 1..20: mol_type = other DNA
ctcaggtactccaaagattc

SEQ ID NO: 12 - DNA, 20 bp, synthetic construct
  misc_feature 1..20: note = B2M gRNA-12
  source 1..20: mol_type = other DNA
gctactctctctttctggcc

SEQ ID NO: 13 - DNA, 20 bp, synthetic construct
  misc_feature 1..20: note = B2M gRNA-13
  source 1..20: mol_type = other DNA
tgggctgtgacaaagtcaca

SEQ ID NO: 14 - DNA, 20 bp, synthetic construct
  misc_feature 1..20: note = B2M gRNA-14
  source 1..20: mol_type = other DNA
acaaagtcacatggttcaca

SEQ ID NO: 15 - DNA, 20 bp, synthetic construct
  misc_feature 1..20: note = B2M gRNA-15
  source 1..20: mol_type = other DNA
gaagttgacttactgaagaa

SEQ ID NO: 16 - DNA, 20 bp, synthetic construct
  misc_feature 1..20: note = B2M gRNA-16
  source 1..20: mol_type = other DNA
tttgactttccattctctgc

SEQ ID NO: 17 - DNA, 20 bp, synthetic construct
  misc_feature 1..20: note = B2M gRNA-17
  source 1..20: mol_type = other DNA
actctctctttctggcctgg

SEQ ID NO: 18 - DNA, 20 bp, synthetic construct
  misc_feature 1..20: note = B2M gRNA-18
  source 1..20: mol_type = other DNA
tggagagagaattgaaaaag

SEQ ID NO: 19 - DNA, 20 bp, synthetic construct
  misc_feature 1..20: note = B2M gRNA-19
  source 1..20: mol_type = other DNA
tcctgaattgctatgtgtct

SEQ ID NO: 20 - DNA, 20 bp, synthetic construct
  misc_feature 1..20: note = B2M gRNA-20
  source 1..20: mol_type = other DNA
ggcatactcatctttttcag

SEQ ID NO: 21 - DNA, 20 bp, synthetic construct
  misc_feature 1..20: note = B2M gRNA-21
  source 1..20: mol_type = other DNA
tcctgaattgctatgtgtct

SEQ ID NO: 22 - DNA, 20 bp, synthetic construct
  misc_feature 1..20: note = B2M gRNA-22
  source 1..20: mol_type = other DNA
gagtagcgcgagcacagcta

SEQ ID NO: 23 - DNA, 20 bp, synthetic construct
  misc_feature 1..20: note = TRAC gRNA-3.4
  source 1..20: mol_type = other DNA
cgtcatgagcagattaaacc

SEQ ID NO: 24 - DNA, 20 bp, synthetic construct
  misc_feature 1..20: note = TRAC gRNA-19.6
  source 1..20: mol_type = other DNA
gagaatcaaaatcggtgaat

SEQ ID NO: 25 - DNA, 20 bp, synthetic construct
  misc_feature 1..20: note = CD7 gRNA-11
  source 1..20: mol_type = other DNA
gggaccctgagaagtcgatg

SEQ ID NO: 26 - DNA, 3128 bp, synthetic construct
  misc_feature 1..3128: note = pDA-T7-B2M gRNA 1 (pDA B2M gRNA-M1)
  source 1..3128: mol_type = other DNA
cccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagactcacgctggatagcctccgttttagagctagaaatagcaagttaaaataaggctagtccgttatcaacttgaaaaagtggcaccgagtcggtgctttttttgaattcgagctcggtacctcgcgaatgcatctagatatcggatcccgggcccgtcgactgcagaggcctgcatgcaagcttctagtggcgcctgatgcggtattttctccttacgcatctgtgcggtatttcacaccgcataggccgctgtattctatagtgtcacctaaatggccgcacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggttt

SEQ ID NO: 27 - DNA, 18 bp, synthetic construct
  misc_feature 1..18: note = T7
  source 1..18: mol_type = other DNA
taatacgactcactatag

SEQ ID NO: 28 - DNA, 76 bp, synthetic construct
  misc_feature 1..76: note = gRNA
  source 1..76: mol_type = other DNA
gttttagagctagaaatagcaagttaaaataaggctagtccgttatcaacttgaaaaagtggcaccgagtcggtgc

SEQ ID NO: 29 - skipped

SEQ ID NO: 30 - DNA, 22 bp, synthetic construct
  misc_feature 1..22: note = PCR
  source 1..22: mol_type = other DNA
tgcagctggcacgacaggtttc

SEQ ID NO: 31 - DNA, 19 bp, synthetic construct
  misc_feature 1..19: note = PCR
  source 1..19: mol_type = other DNA
cgtcagactgtagcacgtt

SEQ ID NO: 32 - DNA, 20 bp, synthetic construct
  misc_feature 1..20: note = forward primer of Sanger sequencing
  source 1..20: mol_type = other DNA
aacaattgaattcaggagga

SEQ ID NO: 33 - DNA, 19 bp, synthetic construct
  misc_feature 1..19: note = Reverse primers for Sanger sequencing
  source 1..19: mol_type = other DNA
cctcctaagaagcgtagtc

SEQ ID NO: 34 - protein, 244 aa, synthetic construct
  REGION 1..244: note = anti-CD7 scFv H1,
QPVLTQSPSASETPGQRVTISCSGSTSNTGNNYFSWYQQVPGMAPKLLIYKNNLRPSGVPDRFSGSKSGTSASLSISGLRSEDEGDYYCATWDDNLTLVFGGGTKLTVLGGGGSGGGGSGGGGSQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGLGTLVTVSS

SEQ ID NO: 35 - protein, 109 aa, synthetic construct
  REGION 1..109: note = anti-CD7 scFv H1, VL,
QPVLTQSPSASETPGQRVTISCSGSTSNTGNNYFSWYQQVPGMAPKLLIYKNNLRPSGVPDRFSGSKSGTSASLSISGLRSEDEGDYYCATWDDNLTLVFGGGTKLTVL

SEQ ID NO: 36 - protein, 120 aa, synthetic construct
  REGION 1..120: note = anti-CD7 scFv H1, VH,
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGLGTLVTVSS

SEQ ID NO: 37 - protein, 488 aa, synthetic construct
  REGION 1..488: note = CD7H1.BBZ,
MALPVTALLLPLALLLHAARPQPVLTQSPSASETPGQRVTISCSGSTSNTGNNYFSWYQQVPGMAPKLLIYKNNLRPSGVPDRFSGSKSGTSASLSISGLRSEDEGDYYCATWDDNLTLVFGGGTKLTVLGGGGSGGGGSGGGGSQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGLGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 1464 aa, synthetic construct
  REGION 1..1464: note = CD7H1.BBZ,
ATGGCCTTACCAGTGACCGCCTTGCTCCTGCCGCTGGCCTTGCTGCTCCACGCCGCCAGGCCGCAGCCTGTGCTGACTCAGTCACCGTCAGCGTCTGAGACCCCCGGGCAGAGGGTCACCATCTCTTGTTCTGGGAGCACCTCCAACACCGGAAACAATTATTTTTCTTGGTACCAACAAGTCCCAGGAATGGCCCCCAAACTCCTCATTTATAAGAACAATCTGCGGCCCTCAGGGGTCCCTGACCGATTCTCTGGCTCCAAGTCTGGCACCTCAGCCTCCCTGTCCATCAGTGGACTCCGGTCCGAGGATGAGGGTGATTATTATTGTGCAACATGGGATGACAACTTGACTCTGGTGTTCGGCGGAGGGACCAAGCTGACCGTCCTAGGTGGTGGTGGTTCTGGCGGCGGCGGCTCCGGAGGTGGTGGATCCCAGGTGCAGCTGCAGGAGTCGGGGGGAGGCTTGGTACAGCCTGGGGGGTCCCTGAGACTCTCCTGTGCAGCCTCTGGATTCACCTTTAGCAGCTATGCCATGAGCTGGGTCCGCCAGGCTCCAGGGAAGGGGCTGGAGTGGGTCTCAGCTATTAGTGGTAGTGGTGGTAGCACATACTACGCAGACTCCGTGAAGGGCCGGTTCACCATCTCCAGAGACAATTCCAAGAACACGCTGTATCTGCAAATGAACAGCCTGAGAGCCGAGGACACGGCCGTATATTACTGTGCGAAAGGGTACAGTGGCTACCCCGCAGTGCTTGACTACTGGGGCCTGGGAACCCTGGTCACTGTCTCCTCAACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACAAGCAGGGCCAGAACCAGCTCTATAACGAGCTCAATCTAGGACGAAGAGAGGAGTACGACGTTTTGGACAAGAGACGTGGCCGGGACCCTGAGATGGGGGGAAAGCCGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGC

SEQ ID NO: 39 - protein, 244 aa, synthetic construct
  REGION 1..244: note = anti-CD7 scFv H4,
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGDWVFGGGTKLTVLGGGGSGGGGSGGGGSQVQLVQSGGGLVQPGGSLRLSCAASGFTFSTYWMSWVRQAPGKGLEWVANINQDGTEKYYVDSVKGRFTISRDNAKNSLYLQMNSLRAEDTAVYYCAREPGIALDYWGQGTLVTVSS

SEQ ID NO: 40 - protein, 112 aa, synthetic construct
  REGION 1..112: note = anti-CD7 scFv H4, VL,
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGDWVFGGGTKLTVL

SEQ ID NO: 41 - protein, 117 aa, synthetic construct
  REGION 1..117: note = anti-CD7 scFv H4, VH,
QVQLVQSGGGLVQPGGSLRLSCAASGFTFSTYWMSWVRQAPGKGLEWVANINQDGTEKYYVDSVKGRFTISRDNAKNSLYLQMNSLRAEDTAVYYCAREPGIALDYWGQGTLVTVSS

SEQ ID NO: 42 - protein, 488 aa, synthetic construct
  REGION 1..488: note = CD7H4.BBZ,
MALPVTALLLPLALLLHAARPQSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGDWVFGGGTKLTVLGGGGSGGGGSGGGGSQVQLVQSGGGLVQPGGSLRLSCAASGFTFSTYWMSWVRQAPGKGLEWVANINQDGTEKYYVDSVKGRFTISRDNAKNSLYLQMNSLRAEDTAVYYCAREPGIALDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 248 aa, synthetic construct
  REGION 1..248: note = anti-CD7 scFv H6,
QSALTQPPSASGSPGQSVTISCTGTSSDVGGYNYVSWYQQHPGKAPKLMIYEVRKRPSGVPDRFSGSKSGNTASLTVSGLQADDEADYYCSSYAGSNNLGFGGGTKLTVLGGGGSGGGGSGGGGSEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDPDPGYFDWVAGDYWGQGTLVTVSS

SEQ ID NO: 44 - protein, 110 aa, synthetic construct
  REGION 1..110: note = anti-CD7 scFv H6, VL,
QSALTQPPSASGSPGQSVTISCTGTSSDVGGYNYVSWYQQHPGKAPKLMIYEVRKRPSGVPDRFSGSKSGNTASLTVSGLQADDEADYYCSSYAGSNNLGFGGGTKLTVL

SEQ ID NO: 45 - protein, 123 aa, synthetic construct
  REGION 1..123: note = anti-CD7 scFv H6, VH,
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDPDPGYFDWVAGDYWGQGTLVTVSS

SEQ ID NO: 46 - protein, 492 aa, synthetic construct
  REGION 1..492: note = CD7H6.BBZ,
MALPVTALLLPLALLLHAARPQSALTQPPSASGSPGQSVTISCTGTSSDVGGYNYVSWYQQHPGKAPKLMIYEVRKRPSGVPDRFSGSKSGNTASLTVSGLQADDEADYYCSSYAGSNNLGFGGGTKLTVLGGGGSGGGGSGGGGSEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDPDPGYFDWVAGDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 244 aa, synthetic construct
  REGION 1..244: note = anti-CD7 scFv H8,
QSVLTQPPSVSEAPRQRVTISCSGSSSNIGNNAVNWYQQLPGKAPKLLIYYDDLLPSGVSDRFSGSKSGTSASLTISGLQSEDGADYYCASWDDSLNGRVFGGGTKLTVLGGGGSGGGGSGGGGSQVQLQESGAGLLKPSETLSLTCAVYGGSISGYYWSWIRQPPGKGLEWIGEINHSGSTNYNPSLKSRVTISVDTSKNQFSLKLSSVTAADTAVYYCARGGGSSGYYLDYWGQGALVTVSS

SEQ ID NO: 48 - protein, 110 aa, synthetic construct
  REGION 1..110: note = anti-CD7 scFv H8, VL,
QSVLTQPPSVSEAPRQRVTISCSGSSSNIGNNAVNWYQQLPGKAPKLLIYYDDLLPSGVSDRFSGSKSGTSASLTISGLQSEDGADYYCASWDDSLNGRVFGGGTKLTVL

SEQ ID NO: 49 - protein, 119 aa, synthetic construct
  REGION 1..119: note = anti-CD7 scFv H8, VH,
QVQLQESGAGLLKPSETLSLTCAVYGGSISGYYWSWIRQPPGKGLEWIGEINHSGSTNYNPSLKSRVTISVDTSKNQFSLKLSSVTAADTAVYYCARGGGSSGYYLDYWGQGALVTVSS

SEQ ID NO: 50 - protein, 488 aa, synthetic construct
  REGION 1..488: note = CD7H8.BBZ,
MALPVTALLLPLALLLHAARPQSVLTQPPSVSEAPRQRVTISCSGSSSNIGNNAVNWYQQLPGKAPKLLIYYDDLLPSGVSDRFSGSKSGTSASLTISGLQSEDGADYYCASWDDSLNGRVFGGGTKLTVLGGGGSGGGGSGGGGSQVQLQESGAGLLKPSETLSLTCAVYGGSISGYYWSWIRQPPGKGLEWIGEINHSGSTNYNPSLKSRVTISVDTSKNQFSLKLSSVTAADTAVYYCARGGGSSGYYLDYWGQGALVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 247 aa, synthetic construct
  REGION 1..247: note = anti-CD7 scFv H9,
QSVLTQPPSASGAPGERVTISCSGSSSNIGSNYVYWYQQLPGTAPKLLIYNSNQRPSGVPDRFSGSKSGASASLAISGLRSEDEADYYCAAWDDSLSGPVFGGGTKVTVLGGGGSGGGGSGGGGSEVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARGVGLWFGEPYFDYWGQGTLVTVSS

SEQ ID NO: 52 - protein, 110 aa, synthetic construct
  REGION 1..110: note = anti-CD7 scFv H9, VL,
QSVLTQPPSASGAPGERVTISCSGSSSNIGSNYVYWYQQLPGTAPKLLIYNSNQRPSGVPDRFSGSKSGASASLAISGLRSEDEADYYCAAWDDSLSGPVFGGGTKVTVL

SEQ ID NO: 53 - protein, 122 aa, synthetic construct
  REGION 1..122: note = anti-CD7 scFv H9, VH,
EVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARGVGLWFGEPYFDYWGQGTLVTVSS

SEQ ID NO: 54 - protein, 491 aa, synthetic construct
  REGION 1..491: note = CD7H9.BBZ,
MALPVTALLLPLALLLHAARPQSVLTQPPSASGAPGERVTISCSGSSSNIGSNYVYWYQQLPGTAPKLLIYNSNQRPSGVPDRFSGSKSGASASLAISGLRSEDEADYYCAAWDDSLSGPVFGGGTKVTVLGGGGSGGGGSGGGGSEVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARGVGLWFGEPYFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 246 aa, synthetic construct
  REGION 1..246: note = anti-CD7 scFv H10,
QSALTQPASVSGSPGQSITISCTGTSSDVGGSKYVSWYQQHPGKAPKLMIYDVSYRPSGVSDRFSGSKSGNTASLTISGLQAEDEADYYCSSYTSNSNLPLFGGGTKVTVLGGGGSGGGGSGGGGSQLQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 56 - protein, 111 aa, synthetic construct
  REGION 1..111: note = anti-CD7 scFv H10, VL,
QSALTQPASVSGSPGQSITISCTGTSSDVGGSKYVSWYQQHPGKAPKLMIYDVSYRPSGVSDRFSGSKSGNTASLTISGLQAEDEADYYCSSYTSNSNLPLFGGGTKVTVL

SEQ ID NO: 57 - protein, 120 aa, synthetic construct
  REGION 1..120: note = anti-CD7 scFv H10, VH,
QLQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 58 - protein, 490 aa, synthetic construct
  REGION 1..490: note = CD7H10.BBZ,
MALPVTALLLPLALLLHAARPQSALTQPASVSGSPGQSITISCTGTSSDVGGSKYVSWYQQHPGKAPKLMIYDVSYRPSGVSDRFSGSKSGNTASLTISGLQAEDEADYYCSSYTSNSNLPLFGGGTKVTVLGGGGSGGGGSGGGGSQLQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 245 aa, synthetic construct
  REGION 1..245: note = anti-CD7 scFv H12,
QPVLTQPPSASGTPGQRVIISCSGSGSNIGSNTVTWYRQFPGAAPKTLIYNSKLRPSWVPDRFTASKSGTSASLAISGLQSEDEADYYCAAWDDSLNGVVFGGGTKLTVLGGGGSGGGGSGGGGSEVHLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 60 - protein, 110 aa, synthetic construct
  REGION 1..110: note = anti-CD7 scFv H12, VL,
QPVLTQPPSASGTPGQRVIISCSGSGSNIGSNTVTWYRQFPGAAPKTLIYNSKLRPSWVPDRFTASKSGTSASLAISGLQSEDEADYYCAAWDDSLNGVVFGGGTKLTVL

SEQ ID NO: 61 - protein, 120 aa, synthetic construct
  REGION 1..120: note = anti-CD7 scFv H12, VH,
EVHLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 62 - protein, 489 aa, synthetic construct
  REGION 1..489: note = CD7H12.BBZ,
MALPVTALLLPLALLLHAARPQPVLTQPPSASGTPGQRVIISCSGSGSNIGSNTVTWYRQFPGAAPKTLIYNSKLRPSWVPDRFTASKSGTSASLAISGLQSEDEADYYCAAWDDSLNGVVFGGGTKLTVLGGGGSGGGGSGGGGSEVHLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 258 aa, synthetic construct
  REGION 1..258: note = anti-CD7 scFv H13,
QSVVTQPPSASGTPGQRVTISCSGSSSNIGSNSVNWYQQLPGTAPKLLIYNNNQRPSGVPDRFSGSKSGTSASLAISGLQSEDEADYYCATWDDSLNGNVVFGGGTKVTVLGGGGSGGGGSGGGGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDQGVVPAAEIPYYDFWSGPPFDPWGQGTLVTVSS

SEQ ID NO: 64 - protein, 111 aa, synthetic construct
  REGION 1..111: note = anti-CD7 scFv H13, VL,
QSVVTQPPSASGTPGQRVTISCSGSSSNIGSNSVNWYQQLPGTAPKLLIYNNNQRPSGVPDRFSGSKSGTSASLAISGLQSEDEADYYCATWDDSLNGNVVFGGGTKVTVL

SEQ ID NO: 65 - protein, 132 aa, synthetic construct
  REGION 1..132: note = anti-CD7 scFv H13, VH,
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDQGVVPAAEIPYYDFWSGPPFDPWGQGTLVTVSS

SEQ ID NO: 66 - protein, 502 aa, synthetic construct
  REGION 1..502: note = CD7H13.BBZ,
MALPVTALLLPLALLLHAARPQSVVTQPPSASGTPGQRVTISCSGSSSNIGSNSVNWYQQLPGTAPKLLIYNNNQRPSGVPDRFSGSKSGTSASLAISGLQSEDEADYYCATWDDSLNGNVVFGGGTKVTVLGGGGSGGGGSGGGGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDQGVVPAAEIPYYDFWSGPPFDPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 244 aa, synthetic construct
  REGION 1..244: note = anti-CD7 scFv H17,
QSALTQPPSVSGSPGQSVSISCTGSSSDIGASSRVSWYLQPPGKAPQLIIFGVNNRPSGVPDRFSGSKSGNTASLTISGLRAEDEADYYCSSHSSGNTLLFGGGTKLTVLGGGSGGGGSGGGGSQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 68 - protein, 110 aa, synthetic construct
  REGION 1..110: note = anti-CD7 scFv H17, VL,
QSALTQPPSVSGSPGQSVSISCTGSSSDIGASSRVSWYLQPPGKAPQLIIFGVNNRPSGVPDRFSGSKSGNTASLTISGLRAEDEADYYCSSHSSGNTLLFGGGTKLTVL

SEQ ID NO: 69 - protein, 120 aa, synthetic construct
  REGION 1..120: note = anti-CD7 scFv H17, VH,
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 70 - protein, 488 aa, synthetic construct
  REGION 1..488: note = CD7H17.BBZ,
MALPVTALLLPLALLLHAARPQSALTQPPSVSGSPGQSVSISCTGSSSDIGASSRVSWYLQPPGKAPQLIIFGVNNRPSGVPDRFSGSKSGNTASLTISGLRAEDEADYYCSSHSSGNTLLFGGGTKLTVLGGGSGGGGSGGGGSQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 248 aa, synthetic construct
  REGION 1..248: note = anti-CD7 scFv H18,
QSALTQPPSASGSPGQSVTISCTGTSSDVGGYTYVSWYQQHPGKAPKLMIYEVSKRPSGVPDRFSGSKSGNTASLTVSGLQAEDEADYYCSSYAGSNNVVFGGGTELTVLGGGGSGGGGSGGGGSEVQLVESGGGLVQPGGSLRLSCAASGFTFSSYAMSWVRQAPGKGLEWFSAISGSGGSTYYADSVKGRFTISRDNSKNTLYLQMNSLRAEDTAVYYCAKDPDPGYFDWVAGDYWGQGTLVTVSS

SEQ ID NO: 72 - protein, 110 aa, synthetic construct
  REGION 1..110: note = anti-CD7 scFv H18, VL,
QSALTQPPSASGSPGQSVTISCTGTSSDVGGYTYVSWYQQHPGKAPKLMIYEVSKRPSGVPDRFSGSKSGNTASLTVSGLQAEDEADYYCSSYAGSNNVVFGGGTELTVL

SEQ ID NO: 73 - protein, 123 aa, synthetic construct
  REGION 1..123: note = anti-CD7 scFv H18, VH,
EVQLVESGGGLVQPGGSLRLSCAASGFTFSSYAMSWVRQAPGKGLEWFSAISGSGGSTYYADSVKGRFTISRDNSKNTLYLQMNSLRAEDTAVYYCAKDPDPGYFDWVAGDYWGQGTLVTVSS

SEQ ID NO: 74 - protein, 492 aa, synthetic construct
  REGION 1..492: note = CD7H18.BBZ,
MALPVTALLLPLALLLHAARPQSALTQPPSASGSPGQSVTISCTGTSSDVGGYTYVSWYQQHPGKAPKLMIYEVSKRPSGVPDRFSGSKSGNTASLTVSGLQAEDEADYYCSSYAGSNNVVFGGGTELTVLGGGGSGGGGSGGGGSEVQLVESGGGLVQPGGSLRLSCAASGFTFSSYAMSWVRQAPGKGLEWFSAISGSGGSTYYADSVKGRFTISRDNSKNTLYLQMNSLRAEDTAVYYCAKDPDPGYFDWVAGDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 245 aa, synthetic construct
  REGION 1..245: note = anti-CD7 scFv H5,
DIRLTQSPSTVSASIGDRVTITCRASQGISNWLTWYQQKPGKAPKLLIYSASTLQSGVPSRFSGSGSGTDFTLTISSLQPEDFATYYCQQANNLRTLTFGGGTKVDIKGGGGSGGGGSGGGGSQVQLVQSGAEVKKPGESLRISCKGSGYSFTSYWISWVRQMPGKGLEWMGRIDPSDSYTNYSPSFQGHVTISADKSISTAYLQWSSLKASDTAMYYCARHPVTNYYYYGMDVWGQGTTVTVSS

SEQ ID NO: 76 - protein, 108 aa, synthetic construct
  REGION 1..108: note = anti-CD7 scFv H5, VL,
DIRLTQSPSTVSASIGDRVTITCRASQGISNWLTWYQQKPGKAPKLLIYSASTLQSGVPSRFSGSGSGTDFTLTISSLQPEDFATYYCQQANNLRTLTFGGGTKVDIK

SEQ ID NO: 77 - protein, 122 aa, synthetic construct
  REGION 1..122: note = anti-CD7 scFv H5, VH,
QVQLVQSGAEVKKPGESLRISCKGSGYSFTSYWISWVRQMPGKGLEWMGRIDPSDSYTNYSPSFQGHVTISADKSISTAYLQWSSLKASDTAMYYCARHPVTNYYYYGMDVWGQGTTVTVSS

SEQ ID NO: 78 - protein, 489 aa, synthetic construct
  REGION 1..489: note = CD7H5.BBZ,
MALPVTALLLPLALLLHAARPDIRLTQSPSTVSASIGDRVTITCRASQGISNWLTWYQQKPGKAPKLLIYSASTLQSGVPSRFSGSGSGTDFTLTISSLQPEDFATYYCQQANNLRTLTFGGGTKVDIKGGGGSGGGGSGGGGSQVQLVQSGAEVKKPGESLRISCKGSGYSFTSYWISWVRQMPGKGLEWMGRIDPSDSYTNYSPSFQGHVTISADKSISTAYLQWSSLKASDTAMYYCARHPVTNYYYYGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 242 aa, synthetic construct
  REGION 1..242: note = anti-CD7 scFv H7,
DIVMTQSPSSLSASVGDRVTITCRASQSISSYLNWYQQKPGKAPKLLIYAASSLQSGVPSRFSGSGSGTDFTLTISSLQPEDFATYYCQQSYSTLPYTFGQGTKLEIKGGGGSGGGGSGGGGSEVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCATDYGELHDAFDIWGQGTTVTVSS

SEQ ID NO: 80 - protein, 108 aa, synthetic construct
  REGION 1..108: note = anti-CD7 scFv H7, VL,
DIVMTQSPSSLSASVGDRVTITCRASQSISSYLNWYQQKPGKAPKLLIYAASSLQSGVPSRFSGSGSGTDFTLTISSLQPEDFATYYCQQSYSTLPYTFGQGTKLEIK

SEQ ID NO: 81 - protein, 119 aa, synthetic construct
  REGION 1..119: note = anti-CD7 scFv H7, VH,
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCATDYGELHDAFDIWGQGTTVTVSS

SEQ ID NO: 82 - protein, 486 aa, synthetic construct
  REGION 1..486: note = CD7H7.BBZ,
MALPVTALLLPLALLLHAARPDIVMTQSPSSLSASVGDRVTITCRASQSISSYLNWYQQKPGKAPKLLIYAASSLQSGVPSRFSGSGSGTDFTLTISSLQPEDFATYYCQQSYSTLPYTFGQGTKLEIKGGGGSGGGGSGGGGSEVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCATDYGELHDAFDIWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 244 aa, synthetic construct
  REGION 1..244: note = anti-CD7 scFv H15,
AIQLTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKVEIKGGGGSGGGGSGGGGSEVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATERASGSYEYYFDYWGQGTLVTVSS

SEQ ID NO: 84 - protein, 107 aa, synthetic construct
  REGION 1..107: note = anti-CD7 scFv H15, VL,
AIQLTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKVEIK

SEQ ID NO: 85 - protein, 122 aa, synthetic construct
  REGION 1..122: note = anti-CD7 scFv H15, VH,
EVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATERASGSYEYYFDYWGQGTLVTVSS

SEQ ID NO: 86 - protein, 488 aa, synthetic construct
  REGION 1..488: note = CD7H15.BBZ,
MALPVTALLLPLALLLHAARPAIQLTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKVEIKGGGGSGGGGSGGGGSEVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATERASGSYEYYFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 7 aa, synthetic construct
  REGION 1..7: note = H1 HCDR1; H6 HCDR1; H10 HCDR1; H12 HCDR1; H17 HCDR1; H18 HCDR1
GFTFSSY

SEQ ID NO: 88 - protein, 7 aa, synthetic construct
  REGION 1..7: note = H4 HCDR1
GFTFSTY

SEQ ID NO: 89 - protein, 7 aa, synthetic construct
  REGION 1..7: note = H8 HCDR1
GGSISGY

SEQ ID NO: 90 - protein, 7 aa, synthetic construct
  REGION 1..7: note = H9 HCDR1
GYTFTGY

SEQ ID NO: 91 - protein, 7 aa, synthetic construct
  REGION 1..7: note = H13 HCDR1
GGTFSSY

SEQ ID NO: 92 - protein, 7 aa, synthetic construct
  REGION 1..7: note = H5 HCDR1
GYSFTSY

SEQ ID NO: 93 - protein, 7 aa, synthetic construct
  REGION 1..7: note = H7 HCDR1
GFTVSSN

SEQ ID NO: 94 - protein, 7 aa, synthetic construct
  REGION 1..7: note = H15 HCDR1
GYTLTEL

SEQ ID NO: 95 - protein, 6 aa, synthetic construct
  REGION 1..6: note = H1 HCDR2; H6 HCDR2; H10 HCDR2; H12 HCDR2; H17 HCDR2; H18 HCDR2
SGSGGS

SEQ ID NO: 96 - protein, 6 aa, synthetic construct
  REGION 1..6: note = H4 HCDR2
NQDGTE

SEQ ID NO: 97 - protein, 5 aa, synthetic construct
  REGION 1..5: note = H8 HCDR2
NHSGS

SEQ ID NO: 98 - protein, 6 aa, synthetic construct
  REGION 1..6: note = H9 HCDR2
NPNSGG

SEQ ID NO: 99 - protein, 6 aa, synthetic construct
  REGION 1..6: note = H13 HCDR2
IPIFGT

SEQ ID NO: 100 - protein, 6 aa, synthetic construct
  REGION 1..6: note = H5 HCDR2
DPSDSY

SEQ ID NO: 101 - protein, 5 aa, synthetic construct
  REGION 1..5: note = H7 HCDR2
YSGGS

SEQ ID NO: 102 - protein, 6 aa, synthetic construct
  REGION 1..6: note = H15 HCDR2
DPEDGE

SEQ ID NO: 103 - protein, 11 aa, synthetic construct
  REGION 1..11: note = H1 HCDR3; H10 HCDR3; H12 HCDR3; H17 HCDR3
GYSGYPAVLDY

SEQ ID NO: 104 - protein, 8 aa, synthetic construct
  REGION 1..8: note = H4 HCDR3
EPGIALDY

SEQ ID NO: 105 - protein, 14 aa, synthetic construct
  REGION 1..14: note = H6 HCDR3
DPDPGYFDWVAGDY

SEQ ID NO: 106 - protein, 11 aa, synthetic construct
  REGION 1..11: note = H8 HCDR3
GGGSSGYYLDY

SEQ ID NO: 107 - protein, 13 aa, synthetic construct
  REGION 1..13: note = H9 HCDR3
GVGLWFGEPYFDY

SEQ ID NO: 108 - protein, 23 aa, synthetic construct
  REGION 1..23: note = H13 HCDR3
DQGVVPAAEIPYYDFWSGPPFDP

SEQ ID NO: 109 - protein, 15 aa, synthetic construct
  REGION 1..15: note = H18 HCDR3
KDPDPGYFDWVAGDY

SEQ ID NO: 110 - protein, 13 aa, synthetic construct
  REGION 1..13: note = H5 HCDR3
HPVTNYYYYGMDV

SEQ ID NO: 111 - protein, 11 aa, synthetic construct
  REGION 1..11: note = H7 HCDR3
DYGELHDAFDI

SEQ ID NO: 112 - protein, 13 aa, synthetic construct
  REGION 1..13: note = H15 HCDR3
ERASGSYEYYFDY

SEQ ID NO: 113 - protein, 13 aa, synthetic construct
  REGION 1..13: note = H1 LCDR1
SGSTSNTGNNYFS

SEQ ID NO: 114 - protein, 13 aa, synthetic construct
  REGION 1..13: note = H4 LCDR1
SGSSSNIGNNYVS

SEQ ID NO: 115 - protein, 14 aa, synthetic construct
  REGION 1..14: note = H6 LCDR1
TGTSSDVGGYNYVS

SEQ ID NO: 116 - protein, 13 aa, synthetic construct
  REGION 1..13: note = H8 LCDR1
SGSSSNIGNNAVN

SEQ ID NO: 117 - protein, 13 aa, synthetic construct
  REGION 1..13: note = H9 LCDR1
SGSSSNIGSNYVY

SEQ ID NO: 118 - protein, 14 aa, synthetic construct
  REGION 1..14: note = H10 LCDR1
TGTSSDVGGSKYVS

SEQ ID NO: 119 - protein, 13 aa, synthetic construct
  REGION 1..13: note = H12 LCDR1
SGSGSNIGSNTVT

SEQ ID NO: 120 - protein, 13 aa, synthetic construct
  REGION 1..13: note = H13 LCDR1
SGSSSNIGSNSVN

SEQ ID NO: 121 - protein, 14 aa, synthetic construct
  REGION 1..14: note = H17 LCDR1
TGSSSDIGASSRVS

SEQ ID NO: 122 - protein, 14 aa, synthetic construct
  REGION 1..14: note = H18 LCDR1
TGTSSDVGGYTYVS

SEQ ID NO: 123 - protein, 11 aa, synthetic construct
  REGION 1..11: note = H5 LCDR1
RASQGISNWLT

SEQ ID NO: 124 - protein, 11 aa, synthetic construct
  REGION 1..11: note = H7 LCDR1
RASQSISSYLN

SEQ ID NO: 125 - protein, 11 aa, synthetic construct
  REGION 1..11: note = H15 LCDR1
RASQGISSWLA

SEQ ID NO: 126 - protein, 7 aa, synthetic construct
  REGION 1..7: note = H1 LCDR2
KNNLRPS

SEQ ID NO: 127 - protein, 7 aa, synthetic construct
  REGION 1..7: note = H4 LCDR2
DNNKRPS

SEQ ID NO: 128 - protein, 7 aa, synthetic construct
  REGION 1..7: note = H6 LCDR2
EVRKRPS

SEQ ID NO: 129 - protein, 7 aa, synthetic construct
  REGION 1..7: note = H8 LCDR2
YDDLLPS

SEQ ID NO: 130 - protein, 7 aa, synthetic construct
  REGION 1..7: note = H9 LCDR2
NSNQRPS

SEQ ID NO: 131 - protein, 7 aa, synthetic construct
  REGION 1..7: note = H10 LCDR2
DVSYRPS

SEQ ID NO: 132 - protein, 7 aa, synthetic construct
  REGION 1..7: note = H12 LCDR2
NSKLRPS

SEQ ID NO: 133 - protein, 7 aa, synthetic construct
  REGION 1..7: note = H13 LCDR2
NNNQRPS

SEQ ID NO: 134 - protein, 7 aa, synthetic construct
  REGION 1..7: note = H17 LCDR2
GVNNRPS

SEQ ID NO: 135 - protein, 7 aa, synthetic construct
  REGION 1..7: note = H18 LCDR2
EVSKRPS

SEQ ID NO: 136 - protein, 7 aa, synthetic construct
  REGION 1..7: note = H5 LCDR2
SASTLQS

SEQ ID NO: 137 - protein, 7 aa, synthetic construct
  REGION 1..7: note = H7 LCDR2; H15 LCDR2
AASSLQS

SEQ ID NO: 138 - protein, 10 aa, synthetic construct
  REGION 1..10: note = H1 LCDR3
ATWDDNLTLV

SEQ ID NO: 139 - protein, 13 aa, synthetic construct
  REGION 1..13: note = H4 LCDR3
GTWDSSLSAGDWV

SEQ ID NO: 140 - protein, 10 aa, synthetic construct
  REGION 1..10: note = H6 LCDR3
SSYAGSNNLG

SEQ ID NO: 141 - protein, 11 aa, synthetic construct
  REGION 1..11: note = H8 LCDR3
ASWDDSLNGRV

SEQ ID NO: 142 - protein, 11 aa, synthetic construct
  REGION 1..11: note = H9 LCDR3
AAWDDSLSGPV

SEQ ID NO: 143 - protein, 11 aa, synthetic construct
  REGION 1..11: note = H10 LCDR3
SSYTSNSNLPL

SEQ ID NO: 144 - protein, 11 aa, synthetic construct
  REGION 1..11: note = H12 LCDR3
AAWDDSLNGVV

SEQ ID NO: 145 - protein, 12 aa, synthetic construct
  REGION 1..12: note = H13 LCDR3
ATWDDSLNGNVV

SEQ ID NO: 146 - protein, 10 aa, synthetic construct
  REGION 1..10: note = H17 LCDR3
SSHSSGNTLL

SEQ ID NO: 147 - protein, 10 aa, synthetic construct
  REGION 1..10: note = H18 LCDR3
SSYAGSNNVV

SEQ ID NO: 148 - protein, 10 aa, synthetic construct
  REGION 1..10: note = H5 LCDR3
QQANNLRTLT

SEQ ID NO: 149 - protein, 10 aa, synthetic construct
  REGION 1..10: note = H7 LCDR3
QQSYSTLPYT

SEQ ID NO: 150 - protein, 9 aa, synthetic construct
  REGION 1..9: note = H15 LCDR3
QQANSFPYT

SEQ ID NO: 151 - protein, 21 aa, synthetic construct
  REGION 1..21: note = CD8,
MALPVTALLLPLALLLHAARP

SEQ ID NO: 152 - DNA, 63 bp, synthetic construct
  misc_feature 1..63: note = CD8,
  source 1..63: mol_type = other DNA
atggccttaccagtgaccgccttgctcctgccgctggccttgctgctccacgccgccaggccg

SEQ ID NO: 153 - protein, 45 aa, synthetic construct
  REGION 1..45: note = CD8,
TTTPAPRPPTPAPTIASQPLSLRPEACRPAAGGAVHTRGLDFACD

SEQ ID NO: 154 - DNA, 135 bp, synthetic construct
  misc_feature 1..135: note = CD8,
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 155 - protein, 24 aa, synthetic construct
  REGION 1..24: note = CD8,
IYIWAPLAGTCGVLLLSLVITLYC

SEQ ID NO: 156 - DNA, 72 bp, synthetic construct
  misc_feature 1..72: note = CD8,
  source 1..72: mol_type = other DNA
atctacatctgggcgcccttggccgggacttgtggggtccttctcctgtcactggttatcaccctttactgc

SEQ ID NO: 157 - protein, 42 aa, synthetic construct
  REGION 1..42: note = 4-1BB,
KRGRKKLLYIFKQPFMRPVQTTQEEDGCSCRFPEEEEGGCEL

SEQ ID NO: 158 - DNA, 126 bp, synthetic construct
  misc_feature 1..126: note = 4-1BB,
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 159 - protein, 112 aa, synthetic construct
  REGION 1..112: note = CD3-zeta,
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - DNA, 336 bp, synthetic construct
  misc_feature 1..336: note = CD3-zeta,
  source 1..336: mol_type = other DNA
agagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 161 - protein, 371 aa, synthetic construct
  REGION 1..371: note = A40C2828,
MALPVTALLLPLALLLHAARPQVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSGGGGSGGGGSGGGGSDIQMTQSPSSLSASVGDRVTITCSASSSVSYMLWFQQKPGKAPKLLIYSTSNLASGVPSRFSGSGSGTDFTLTISSLQPEDFATYYCQQRTFYPYTFGGGTKVEIKRTASIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 162 - DNA, 1116 bp, synthetic construct
  misc_feature 1..1116: note = A40C2828,
  source 1..1116: mol_type = other DNA
atggccttaccagtgaccgccttgctcctgccgctggccttgctgctccacgccgccaggccgcaggtccagctggtacaatctggtgctgaagtgaagaaaccaggagcgtcggtgaaggtctcatgcacagcctcaggcttcaacatcaaggactactacgtgcactgggtcaagcaagctcccggacaggggctggagtggatggggagaattgacccggaagacggcgactcgaaatacgcgccgaagttccagggcaaggccaccatgacagccgataccagtacatcgaccgtttacatggagctgtccagcctccgctcagaggataccgcggtatactactgtacgacctcctactacgtcggaacgtacggttactggggccagggtaccctggtgacagtgtcctcggggggcggcgggtctggcgggggaggcagcgggggcggcgggtcggacatccagatgactcaatccccctcttccctgagtgcgtccgtgggtgacagggtgacgatcacttgtagcgcttcgtcctcggtgtcgtatatgctgtggttccagcagaagcccggtaaggcccccaaactgctcatttattccaccagcaacctcgccagtggcgtacccagtcgcttctccggtagtggctccggtaccgatttcacactgaccatctccagtctgcaacctgaagacttcgcaacgtactattgtcagcagcgcacattctatccctatacgtttgggggcgggaccaaggtcgagatcaagcgcacagctagcattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctaa

SEQ ID NO: 163 - protein, 243 aa, synthetic construct
  REGION 1..243: note = Anti-CD40 scFv A40,
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSGGGGSGGGGSGGGGSDIQMTQSPSSLSASVGDRVTITCSASSSVSYMLWFQQKPGKAPKLLIYSTSNLASGVPSRFSGSGSGTDFTLTISSLQPEDFATYYCQQRTFYPYTFGGGTKVEIKRTAS

SEQ ID NO: 164 - protein, 39 aa, synthetic construct
  REGION 1..39: note = CD28 hinge,
IEVMYPPPYLDNEKSNGTIIHVKGKHLCPSPLFPGPSKP

SEQ ID NO: 165 - protein, 27 aa, synthetic construct
  REGION 1..27: note = CD28 TM,
FWVLVVVGGVLACYSLLVTVAFIIFWV

SEQ ID NO: 166 - protein, 41 aa, synthetic construct
  REGION 1..41: note = CD28 Intracellular domain,
RSKRSRLLHSDYMNMTPRRPGPTRKHYQPYAPPRDFAAYRS

SEQ ID NO: 167 - DNA, 4112 bp, synthetic construct
  misc_feature 1..4112: note = R196H1A (pAAV-HA gRNA19.6-CD7H1.BBZ-F2A-A40C2828
  source 1..4112: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcaacgcgtagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctatttctagagcgcaagcgccgcagcggcagcggcgccaccaacttctccctgctgaagcaggccggcgacgtggaggagaaccccggccccaagcttatggccttaccagtgaccgccttgctcctgccgctggccttgctgctccacgccgccaggccgcagcctgtgctgactcagtcaccgtcagcgtctgagacccccgggcagagggtcaccatctcttgttctgggagcacctccaacaccggaaacaattatttttcttggtaccaacaagtcccaggaatggcccccaaactcctcatttataagaacaatctgcggccctcaggggtccctgaccgattctctggctccaagtctggcacctcagcctccctgtccatcagtggactccggtccgaggatgagggtgattattattgtgcaacatgggatgacaacttgactctggtgttcggcggagggaccaagctgaccgtcctaggtggtggtggttctggcggcggcggctccggaggtggtggatcccaggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggcctgggaaccctggtcactgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgtgaaacagactttgaattttgaccttctcaagttggcgggagacgtggagtccaacccagggccgatggccttaccagtgaccgccttgctcctgccgctggccttgctgctccacgccgccaggccgcaggtccagctggtacaatctggtgctgaagtgaagaaaccaggagcgtcggtgaaggtctcatgcacagcctcaggcttcaacatcaaggactactacgtgcactgggtcaagcaagctcccggacaggggctggagtggatggggagaattgacccggaagacggcgactcgaaatacgcgccgaagttccagggcaaggccaccatgacagccgataccagtacatcgaccgtttacatggagctgtccagcctccgctcagaggataccgcggtatactactgtacgacctcctactacgtcggaacgtacggttactggggccagggtaccctggtgacagtgtcctcggggggcggcgggtctggcgggggaggcagcgggggcggcgggtcggacatccagatgactcaatccccctcttccctgagtgcgtccgtgggtgacagggtgacgatcacttgtagcgcttcgtcctcggtgtcgtatatgctgtggttccagcagaagcccggtaaggcccccaaactgctcatttattccaccagcaacctcgccagtggcgtacccagtcgcttctccggtagtggctccggtaccgatttcacactgaccatctccagtctgcaacctgaagacttcgcaacgtactattgtcagcagcgcacattctatccctatacgtttgggggcgggaccaaggtcgagatcaagcgcacagctagcattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctaaggatccaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctgaattcactagtcaccgattttgaaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcacacgtgcaactcaggggcctccaaaggtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 168 - DNA, 4327 bp, synthetic construct
  misc_feature 1..4327: note = R3H1A (pAAV-HA gRNA3.4-CD7H1.BBZ-F2A-A40C2828)
  source 1..4327: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcaacgcgtagatcctattaaataaaaggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccaagcttggctctggagccaccaacttctccctgctgaagcaggccggcgacgtggaggagaacccaggcccatctagaatggccttaccagtgaccgccttgctcctgccgctggccttgctgctccacgccgccaggccgcagcctgtgctgactcagtcaccgtcagcgtctgagacccccgggcagagggtcaccatctcttgttctgggagcacctccaacaccggaaacaattatttttcttggtaccaacaagtcccaggaatggcccccaaactcctcatttataagaacaatctgcggccctcaggggtccctgaccgattctctggctccaagtctggcacctcagcctccctgtccatcagtggactccggtccgaggatgagggtgattattattgtgcaacatgggatgacaacttgactctggtgttcggcggagggaccaagctgaccgtcctaggtggtggtggttctggcggcggcggctccggaggtggtggatcccaggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggcctgggaaccctggtcactgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgtgaaacagactttgaattttgaccttctcaagttggcgggagacgtggagtccaacccagggccgatggccttaccagtgaccgccttgctcctgccgctggccttgctgctccacgccgccaggccgcaggtccagctggtacaatctggtgctgaagtgaagaaaccaggagcgtcggtgaaggtctcatgcacagcctcaggcttcaacatcaaggactactacgtgcactgggtcaagcaagctcccggacaggggctggagtggatggggagaattgacccggaagacggcgactcgaaatacgcgccgaagttccagggcaaggccaccatgacagccgataccagtacatcgaccgtttacatggagctgtccagcctccgctcagaggataccgcggtatactactgtacgacctcctactacgtcggaacgtacggttactggggccagggtaccctggtgacagtgtcctcggggggcggcgggtctggcgggggaggcagcgggggcggcgggtcggacatccagatgactcaatccccctcttccctgagtgcgtccgtgggtgacagggtgacgatcacttgtagcgcttcgtcctcggtgtcgtatatgctgtggttccagcagaagcccggtaaggcccccaaactgctcatttattccaccagcaacctcgccagtggcgtacccagtcgcttctccggtagtggctccggtaccgatttcacactgaccatctccagtctgcaacctgaagacttcgcaacgtactattgtcagcagcgcacattctatccctatacgtttgggggcgggaccaaggtcgagatcaagcgcacagctagcattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctaagtcgacaggatccaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctgaattcactagctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgactagttgacacgggaaaaaagcacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 169 - DNA, 4775 bp, synthetic construct
  misc_feature 1..4775: note = R3PGK (pAAV-HA gRNA3.4-PGK-CD7H1.BBZ-F2A-A40C2828)
  source 1..4775: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcaacgcgtagatcctattaaataaaaggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccaagctttaaggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagtctagaatggccttaccagtgaccgccttgctcctgccgctggccttgctgctccacgccgccaggccgcagcctgtgctgactcagtcaccgtcagcgtctgagacccccgggcagagggtcaccatctcttgttctgggagcacctccaacaccggaaacaattatttttcttggtaccaacaagtcccaggaatggcccccaaactcctcatttataagaacaatctgcggccctcaggggtccctgaccgattctctggctccaagtctggcacctcagcctccctgtccatcagtggactccggtccgaggatgagggtgattattattgtgcaacatgggatgacaacttgactctggtgttcggcggagggaccaagctgaccgtcctaggtggtggtggttctggcggcggcggctccggaggtggtggatcccaggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggcctgggaaccctggtcactgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgtgaaacagactttgaattttgaccttctcaagttggcgggagacgtggagtccaacccagggccgatggccttaccagtgaccgccttgctcctgccgctggccttgctgctccacgccgccaggccgcaggtccagctggtacaatctggtgctgaagtgaagaaaccaggagcgtcggtgaaggtctcatgcacagcctcaggcttcaacatcaaggactactacgtgcactgggtcaagcaagctcccggacaggggctggagtggatggggagaattgacccggaagacggcgactcgaaatacgcgccgaagttccagggcaaggccaccatgacagccgataccagtacatcgaccgtttacatggagctgtccagcctccgctcagaggataccgcggtatactactgtacgacctcctactacgtcggaacgtacggttactggggccagggtaccctggtgacagtgtcctcggggggcggcgggtctggcgggggaggcagcgggggcggcgggtcggacatccagatgactcaatccccctcttccctgagtgcgtccgtgggtgacagggtgacgatcacttgtagcgcttcgtcctcggtgtcgtatatgctgtggttccagcagaagcccggtaaggcccccaaactgctcatttattccaccagcaacctcgccagtggcgtacccagtcgcttctccggtagtggctccggtaccgatttcacactgaccatctccagtctgcaacctgaagacttcgcaacgtactattgtcagcagcgcacattctatccctatacgtttgggggcgggaccaaggtcgagatcaagcgcacagctagcattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctaagtcgacaggatccaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctgaattcactagctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgactagttgacacgggaaaaaagcacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 170 - protein, 881 aa, synthetic construct
  REGION 1..881: note = CD7H1.BBZ-F2A-A40C2828,
MALPVTALLLPLALLLHAARPQPVLTQSPSASETPGQRVTISCSGSTSNTGNNYFSWYQQVPGMAPKLLIYKNNLRPSGVPDRFSGSKSGTSASLSISGLRSEDEGDYYCATWDDNLTLVFGGGTKLTVLGGGGSGGGGSGGGGSQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGLGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVKQTLNFDLLKLAGDVESNPGPMALPVTALLLPLALLLHAARPQVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSGGGGSGGGGSGGGGSDIQMTQSPSSLSASVGDRVTITCSASSSVSYMLWFQQKPGKAPKLLIYSTSNLASGVPSRFSGSGSGTDFTLTISSLQPEDFATYYCQQRTFYPYTFGGGTKVEIKRTASIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 171 - DNA, 2646 bp, synthetic construct
  misc_feature 1..2646: note = CD7H1.BBZ-F2A-A40C2828,
  source 1..2646: mol_type = other DNA
atggccttaccagtgaccgccttgctcctgccgctggccttgctgctccacgccgccaggccgcagcctgtgctgactcagtcaccgtcagcgtctgagacccccgggcagagggtcaccatctcttgttctgggagcacctccaacaccggaaacaattatttttcttggtaccaacaagtcccaggaatggcccccaaactcctcatttataagaacaatctgcggccctcaggggtccctgaccgattctctggctccaagtctggcacctcagcctccctgtccatcagtggactccggtccgaggatgagggtgattattattgtgcaacatgggatgacaacttgactctggtgttcggcggagggaccaagctgaccgtcctaggtggtggtggttctggcggcggcggctccggaggtggtggatcccaggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggcctgggaaccctggtcactgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgtgaaacagactttgaattttgaccttctcaagttggcgggagacgtggagtccaacccagggccgatggccttaccagtgaccgccttgctcctgccgctggccttgctgctccacgccgccaggccgcaggtccagctggtacaatctggtgctgaagtgaagaaaccaggagcgtcggtgaaggtctcatgcacagcctcaggcttcaacatcaaggactactacgtgcactgggtcaagcaagctcccggacaggggctggagtggatggggagaattgacccggaagacggcgactcgaaatacgcgccgaagttccagggcaaggccaccatgacagccgataccagtacatcgaccgtttacatggagctgtccagcctccgctcagaggataccgcggtatactactgtacgacctcctactacgtcggaacgtacggttactggggccagggtaccctggtgacagtgtcctcggggggcggcgggtctggcgggggaggcagcgggggcggcgggtcggacatccagatgactcaatccccctcttccctgagtgcgtccgtgggtgacagggtgacgatcacttgtagcgcttcgtcctcggtgtcgtatatgctgtggttccagcagaagcccggtaaggcccccaaactgctcatttattccaccagcaacctcgccagtggcgtacccagtcgcttctccggtagtggctccggtaccgatttcacactgaccatctccagtctgcaacctgaagacttcgcaacgtactattgtcagcagcgcacattctatccctatacgtttgggggcgggaccaaggtcgagatcaagcgcacagctagcattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctaa

SEQ ID NO: 172 - DNA, 500 bp, synthetic construct
  misc_feature 1..500: note = gRNA3.4-(5)
  source 1..500: mol_type = other DNA
gtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggcc

SEQ ID NO: 173 - DNA, 500 bp, synthetic construct
  misc_feature 1..500: note = gRNA3.4-(3)
  source 1..500: mol_type = other DNA
ctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctg

SEQ ID NO: 174 - DNA, 399 bp, synthetic construct
  misc_feature 1..399: note = gRNA19.6-(5)
  source 1..399: mol_type = other DNA
agatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctat

SEQ ID NO: 175 - DNA, 396 bp, synthetic construct
  misc_feature 1..396: note = gRNA19.6-(3)
  source 1..396: mol_type = other DNA
tcaccgattttgaaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagca

SEQ ID NO: 176 - DNA, 511 bp, synthetic construct
  misc_feature 1..511: note = PGK
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 177 - protein, 15 aa, synthetic construct
  REGION 1..15: note = linker 1
GGGGSGGGGSGGGGS

SEQ ID NO: 178 - protein, 14 aa, synthetic construct
  REGION 1..14: note = linker 2
GGGSGGGGSGGGGS

SEQ ID NO: 179 - protein, 22 aa, synthetic construct
  REGION 1..22: note = F2A
VKQTLNFDLLKLAGDVESNPGP

(19) United States
(12) Patent Application Publication
(10) Pub. No.: US 2026/0337563 A1
(43) Pub. Date: Oct. 29, 2026

(54) UNIVERSAL CAR-T CELL TARGETING CD7

(71) Applicant: UTC THERAPEUTICS (SHANGHAI) CO., LTD., Shanghai (CN)

(72) Inventors: Yangbing Zhao, Shanghai (CN); Xiaojun Liu, Shanghai (CN); Gengzhen Zhu, Shanghai (CN)

(73) Assignee: UTC THERAPEUTICS (SHANGHAI) CO., LTD., Shanghai (CN)

(21) Appl. No.: 19/122,702

(22) PCT Filed: Oct. 20, 2023
(86) PCT No.: PCT/CN2023/125577

§ 371 Date: Apr. 18, 2025

(30) Foreign Application Priority Data
Oct. 20, 2022 (WO) PCT/CN2022/126382

(51) Int. Cl. C12N 5/0783; A61K 35/17; A61K 40/11; A61K 40/31; A61P 35/00; C07K 16/28; C12N 15/90

(52) U.S. Cl. CPC C12N 5/0636; A61K 35/17; A61K 40/11; A61K 40/31; A61P 35/00; C07K 16/2803; C07K 16/2875; C12N 15/907; A61K 2239/13; A61K 2239/29; C07K 2317/565; C12N 2510/00

(57) ABSTRACT

The present invention relates to a modified T cell, a preparation method therefor and a use thereof. The expression of endogenous TRAC, B2M and CD7 genes in the T cell is inhibited, and the T cell expresses a chimeric antigen receptor targeting CD7. The modified T cell can be used to treat diseases associated with CD7 expression.

### CROSS-REFERENCE TO RELATED APPLICATIONS

This application claims priority to PCT Application No. PCT/CN2022/126382, filed on Oct. 20, 2022, which is incorporated herein by reference in its entirety.

### TECHNICAL FIELD

The present invention relates to the field of immunotherapy, and in particular to universal CAR-T cells targeting CD7.

### BACKGROUND ART

T-cell acute lymphoblastic leukemia (T-ALL) and T-cell lymphoblastic lymphoma (T-LBL) are highly aggressive T-lineage malignancies. Many patients will still relapse after chemotherapy or even allogeneic hematopoietic stem cell transplantation, and clinical needs are urgently needed. CD7 is highly expressed on the surface of T-ALL/T-LBL cells and is a potential therapeutic target, but the preparation of CD7 CAR-T faces many challenges, including: it is difficult to separate normal T cells from the peripheral blood of such patients, especially when there are a large number of malignant T cells; CAR-T cells targeting CD7 also express CD7 themselves, and the proliferation of CAR-T is limited due to the “fratricide mechanism”.

### SUMMARY OF THE INVENTION

The present invention provides a universal CAR-T cell targeting CD7. The universal CAR-T cell targeting CD7 of the present invention can avoid allogeneic rejection and fratricide, and also has a strong killing effect.

One aspect of the present invention provides a modified T cell, in which the expression of endogenous TRAC, B2M and CD7 genes is suppressed, and the T cell expresses a chimeric antigen receptor targeting CD7, wherein the chimeric antigen receptor comprises a binding domain that specifically binds to CD7, a transmembrane domain and an intracellular signaling domain.

In some embodiments, the binding domain that specifically binds to CD7 comprises a light chain variable region and a heavy chain variable region, the light chain variable region comprises LCDR1, LCDR2 and LCDR3, and the heavy chain variable region comprises HCDR1, HCDR2 and HCDR3; wherein,
LCDR1 has an amino acid sequence selected from SEQ ID NOs: 113-125;
LCDR2 has an amino acid sequence selected from SEQ ID NOs: 126-137;
LCDR3 has an amino acid sequence selected from SEQ ID NOs: 138-150;
HCDR1 has an amino acid sequence selected from SEQ ID NOs: 87-94;
HCDR2 has an amino acid sequence selected from SEQ ID NOs: 95-102; and
HCDR3 has an amino acid sequence selected from SEQ ID NOs: 103-112.

In some embodiments, the light chain variable region and heavy chain variable region of the binding domain that specifically binds to CD7 comprise LCDR1, LCDR2, LCDR3, HCDR1, HCDR2, and HCDR3 selected from the group consisting of:
(a) an LCDR1 having the amino acid sequence shown in SEQ ID NO:113, an LCDR2 having the amino acid sequence shown in SEQ ID NO:126, an LCDR3 having the amino acid sequence shown in SEQ ID NO:138, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(b) an LCDR1 having the amino acid sequence shown in SEQ ID NO:114, an LCDR2 having the amino acid sequence shown in SEQ ID NO:127, an LCDR3 having the amino acid sequence shown in SEQ ID NO:139, an HCDR1 having the amino acid sequence shown in SEQ ID NO:88, an HCDR2 having the amino acid sequence shown in SEQ ID NO:96, and an HCDR3 having the amino acid sequence shown in SEQ ID NO:104;
(c) an LCDR1 having the amino acid sequence shown in SEQ ID NO:115, an LCDR2 having the amino acid sequence shown in SEQ ID NO:128, an LCDR3 having the amino acid sequence shown in SEQ ID NO:140, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:105;
(d) an LCDR1 having the amino acid sequence shown in SEQ ID NO:116, an LCDR2 having the amino acid sequence shown in SEQ ID NO:129, an LCDR3 having the amino acid sequence shown in SEQ ID NO:141, an HCDR1 having the amino acid sequence shown in SEQ ID NO:89, an HCDR2 having the amino acid sequence shown in SEQ ID NO:97, and an HCDR3 having the amino acid sequence shown in SEQ ID NO:106;
(e) an LCDR1 having the amino acid sequence shown in SEQ ID NO:117, an LCDR2 having the amino acid sequence shown in SEQ ID NO:130, an LCDR3 having the amino acid sequence shown in SEQ ID NO:142, an HCDR1 having the amino acid sequence shown in SEQ ID NO:90, an HCDR2 having the amino acid sequence shown in SEQ ID NO:98, and an HCDR3 having the amino acid sequence shown in SEQ ID NO:107;
(f) an LCDR1 having the amino acid sequence shown in SEQ ID NO:118, an LCDR2 having the amino acid sequence shown in SEQ ID NO:131, an LCDR3 having the amino acid sequence shown in SEQ ID NO:143, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(g) an LCDR1 having the amino acid sequence shown in SEQ ID NO:119, an LCDR2 having the amino acid sequence shown in SEQ ID NO:132, an LCDR3 having the amino acid sequence shown in SEQ ID NO:144, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(h) an LCDR1 having the amino acid sequence shown in SEQ ID NO:120, an LCDR2 having the amino acid sequence shown in SEQ ID NO:133, an LCDR3 having the amino acid sequence shown in SEQ ID NO:145, an HCDR1 having the amino acid sequence shown in SEQ ID NO:91, an HCDR2 having the amino acid sequence shown in SEQ ID NO:99, and an HCDR3 having the amino acid sequence shown in SEQ ID NO:108;
(i) an LCDR1 having the amino acid sequence shown in SEQ ID NO:121, an LCDR2 having the amino acid sequence shown in SEQ ID NO:134, an LCDR3 having the amino acid sequence shown in SEQ ID NO:146, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(j) an LCDR1 having the amino acid sequence shown in SEQ ID NO:122, an LCDR2 having the amino acid sequence shown in SEQ ID NO:135, an LCDR3 having the amino acid sequence shown in SEQ ID NO:147, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:109;
(k) an LCDR1 having the amino acid sequence shown in SEQ ID NO:123, an LCDR2 having the amino acid sequence shown in SEQ ID NO:136, an LCDR3 having the amino acid sequence shown in SEQ ID NO:148, an HCDR1 having the amino acid sequence shown in SEQ ID NO:92, an HCDR2 having the amino acid sequence shown in SEQ ID NO:100, and an HCDR3 having the amino acid sequence shown in SEQ ID NO:110;
(l) an LCDR1 having the amino acid sequence shown in SEQ ID NO:124, an LCDR2 having the amino acid sequence shown in SEQ ID NO:137, an LCDR3 having the amino acid sequence shown in SEQ ID NO:149, an HCDR1 having the amino acid sequence shown in SEQ ID NO:93, an HCDR2 having the amino acid sequence shown in SEQ ID NO:101, and an HCDR3 having the amino acid sequence shown in SEQ ID NO:111; and
(m) an LCDR1 having the amino acid sequence shown in SEQ ID NO:125, an LCDR2 having the amino acid sequence shown in SEQ ID NO:137, an LCDR3 having the amino acid sequence shown in SEQ ID NO:150, an HCDR1 having the amino acid sequence shown in SEQ ID NO:94, an HCDR2 having the amino acid sequence shown in SEQ ID NO:102 and an HCDR3 having the amino acid sequence shown in SEQ ID NO:112.

In some embodiments, the binding domain that specifically binds to CD7 comprises a light chain variable region and a heavy chain variable region selected from the group consisting of:
(a) a light chain variable region comprising the amino acid sequence shown in SEQ ID NO:35 and a heavy chain variable region comprising the amino acid sequence shown in SEQ ID NO:36;
(b) a light chain variable region comprising the amino acid sequence shown in SEQ ID NO:40 and a heavy chain variable region comprising the amino acid sequence shown in SEQ ID NO:41;
(c) a light chain variable region comprising the amino acid sequence shown in SEQ ID NO:44 and a heavy chain variable region comprising the amino acid sequence shown in SEQ ID NO:45;
(d) a light chain variable region comprising the amino acid sequence shown in SEQ ID NO:48 and a heavy chain variable region comprising the amino acid sequence shown in SEQ ID NO:49;
(e) a light chain variable region comprising the amino acid sequence shown in SEQ ID NO:52 and a heavy chain variable region comprising the amino acid sequence shown in SEQ ID NO:53;
(f) a light chain variable region comprising the amino acid sequence shown in SEQ ID NO:56 and a heavy chain variable region comprising the amino acid sequence shown in SEQ ID NO:57;
(g) a light chain variable region comprising the amino acid sequence shown in SEQ ID NO:60 and a heavy chain variable region comprising the amino acid sequence shown in SEQ ID NO:61;
(h) a light chain variable region comprising the amino acid sequence shown in SEQ ID NO:64 and a heavy chain variable region comprising the amino acid sequence shown in SEQ ID NO:65;
(i) a light chain variable region comprising the amino acid sequence shown in SEQ ID NO:68 and a heavy chain variable region comprising the amino acid sequence shown in SEQ ID NO:69;
(j) a light chain variable region comprising the amino acid sequence shown in SEQ ID NO:72 and a heavy chain variable region comprising the amino acid sequence shown in SEQ ID NO:73;
(k) a light chain variable region comprising the amino acid sequence shown in SEQ ID NO:76 and a heavy chain variable region comprising the amino acid sequence shown in SEQ ID NO:77;
(l) a light chain variable region comprising the amino acid sequence shown in SEQ ID NO:80 and a heavy chain variable region comprising the amino acid sequence shown in SEQ ID NO:81; and
(m) a light chain variable region comprising the amino acid sequence shown in SEQ ID NO:84 and a heavy chain variable region comprising the amino acid sequence shown in SEQ ID NO:85.

In some embodiments, the binding domain that specifically binds to CD7 is an scFv.

In some embodiments, the transmembrane domain is a CD8 transmembrane domain.

In some embodiments, the intracellular signaling domain comprises a 4-1BB co-stimulatory domain and a CD3 zeta intracellular domain.

In some embodiments, the chimeric antigen receptor further comprises a hinge region between the binding domain and the transmembrane domain.

In some embodiments, the hinge region is a CD8 hinge region.

In some embodiments, the chimeric antigen receptor targeting CD7 comprises an amino acid sequence selected from SEQ ID NO:37, 42, 46, 50, 54, 58, 62, 66, 70, 74, 78, 82, 86.

In some embodiments, the chimeric antigen receptor targeting CD7 further comprises a signal peptide.

In some embodiments, the signal peptide comprised by the chimeric antigen receptor targeting CD7 is a CD8 signal peptide.

In some embodiments, the nucleic acid sequence encoding the chimeric antigen receptor is inserted into the TRAC locus of the T cell.

In some embodiments, the nucleic acid sequence encoding the chimeric antigen receptor is operably linked to a PGK promoter or an endogenous TRAC promoter of the T cell.

In some embodiments, the modified T cells further express a fusion protein of a lymphocyte-antigen presenting cell co-stimulator, wherein the fusion protein comprises a first domain that activates antigen presenting cells (APCs) and a second domain that activates immune effector cells, wherein

The first domain comprises: (i) a ligand that binds to an activating receptor of the APC, or a receptor-binding fragment thereof, or (ii) an antibody that binds to an activating receptor of the APC, or an antigen-binding fragment thereof; and

The second domain comprises: (i) a co-stimulatory receptor of the immune effector cell, or a functional fragment thereof, (ii) a co-stimulatory ligand of the immune effector cell, or a receptor binding fragment thereof, or (iii) an antibody that binds to the co-stimulatory receptor of the immune effector cell, or an antigen-binding fragment thereof.

In some embodiments, the activating receptor of the APC is CD40.

In some embodiments, the first domain is an anti-CD40 antibody or an antigen-binding fragment thereof.

In some embodiments, the first domain is an scFv.

In some embodiments, the immune effector cells are T cells.

In some embodiments, the second domain comprises the intracellular domain of the co-stimulatory receptor.

In some embodiments, the co-stimulatory receptor is CD28.

In some embodiments, the second domain further comprises a transmembrane domain of the co-stimulatory receptor.

In some embodiments, the first domain and the second domain are connected via a CD28 hinge region.

In some embodiments, the lymphocyte-antigen presenting cell co-stimulatory fusion protein comprises the amino acid sequence shown in SEQ ID NO:161.

In some embodiments, the lymphocyte-antigen presenting cell co-stimulatory fusion protein further comprises a signal peptide.

In some embodiments, the fusion protein of the lymphocyte-antigen-presenting cell (APC) co-stimulator comprises a signal peptide that is a CD8 signal peptide.

In some embodiments, the nucleic acid sequence encoding the lymphocyte-antigen presenting cell co-stimulatory fusion protein and the nucleic acid sequence encoding the chimeric antigen receptor targeting CD7 are in the same expression frame or in separate expression frames.

In some embodiments, endogenous TRAC, B2M and CD7 genes are knocked out in the T cells.

Another aspect of the present invention provides a method for producing modified T cells, the method comprising:
(a) suppressing the expression of endogenous TRAC, B2M and CD7 genes in the T cells; and
(b) introducing a polynucleotide sequence comprising a nucleic acid sequence encoding a chimeric antigen receptor targeting CD7 into the T cell, so that the T cell expresses the chimeric antigen receptor, wherein the chimeric antigen receptor targeting CD7 comprises a binding domain that specifically binds to CD7, a transmembrane domain, and an intracellular signaling domain.

In some embodiments, the binding domain that specifically binds to CD7 comprises a light chain variable region and a heavy chain variable region, the light chain variable region comprises LCDR1, LCDR2 and LCDR3, and the heavy chain variable region comprises HCDR1, HCDR2 and HCDR3; wherein,
LCDR1 has an amino acid sequence selected from SEQ ID NOs: 113-125;
LCDR2 has an amino acid sequence selected from SEQ ID NOs: 126-137;
LCDR3 has an amino acid sequence selected from SEQ ID NOs: 138-150;
HCDR1 has an amino acid sequence selected from SEQ ID NOs: 87-94;
HCDR2 has an amino acid sequence selected from SEQ ID NOs: 95-102; and
HCDR3 has an amino acid sequence selected from SEQ ID NOs: 103-112.

In some embodiments, the light chain variable region and heavy chain variable region of the binding domain that specifically binds to CD7 comprise LCDR1, LCDR2, LCDR3, HCDR1, HCDR2, and HCDR3 selected from the group consisting of:
(a) an LCDR1 having the amino acid sequence shown in SEQ ID NO:113, an LCDR2 having the amino acid sequence shown in SEQ ID NO:126, an LCDR3 having the amino acid sequence shown in SEQ ID NO:138, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(b) an LCDR1 having the amino acid sequence shown in SEQ ID NO:114, an LCDR2 having the amino acid sequence shown in SEQ ID NO:127, an LCDR3 having the amino acid sequence shown in SEQ ID NO:139, an HCDR1 having the amino acid sequence shown in SEQ ID NO:88, an HCDR2 having the amino acid sequence shown in SEQ ID NO:96, and an HCDR3 having the amino acid sequence shown in SEQ ID NO:104;
(c) an LCDR1 having the amino acid sequence shown in SEQ ID NO:115, an LCDR2 having the amino acid sequence shown in SEQ ID NO:128, an LCDR3 having the amino acid sequence shown in SEQ ID NO:140, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:105;
(d) an LCDR1 having the amino acid sequence shown in SEQ ID NO:116, an LCDR2 having the amino acid sequence shown in SEQ ID NO:129, an LCDR3 having the amino acid sequence shown in SEQ ID NO:141, an HCDR1 having the amino acid sequence shown in SEQ ID NO:89, an HCDR2 having the amino acid sequence shown in SEQ ID NO:97, and an HCDR3 having the amino acid sequence shown in SEQ ID NO:106;
(e) an LCDR1 having the amino acid sequence shown in SEQ ID NO:117, an LCDR2 having the amino acid sequence shown in SEQ ID NO:130, an LCDR3 having the amino acid sequence shown in SEQ ID NO:142, an HCDR1 having the amino acid sequence shown in SEQ ID NO:90, an HCDR2 having the amino acid sequence shown in SEQ ID NO:98, and an HCDR3 having the amino acid sequence shown in SEQ ID NO:107;
(f) an LCDR1 having the amino acid sequence shown in SEQ ID NO:118, an LCDR2 having the amino acid sequence shown in SEQ ID NO:131, an LCDR3 having the amino acid sequence shown in SEQ ID NO:143, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(g) an LCDR1 having the amino acid sequence shown in SEQ ID NO:119, an LCDR2 having the amino acid sequence shown in SEQ ID NO:132, an LCDR3 having the amino acid sequence shown in SEQ ID NO:144, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(h) an LCDR1 having the amino acid sequence shown in SEQ ID NO:120, an LCDR2 having the amino acid sequence shown in SEQ ID NO:133, an LCDR3 having the amino acid sequence shown in SEQ ID NO:145, an HCDR1 having the amino acid sequence shown in SEQ ID NO:91, an HCDR2 having the amino acid sequence shown in SEQ ID NO:99, and an HCDR3 having the amino acid sequence shown in SEQ ID NO:108;
(i) an LCDR1 having the amino acid sequence shown in SEQ ID NO:121, an LCDR2 having the amino acid sequence shown in SEQ ID NO:134, an LCDR3 having the amino acid sequence shown in SEQ ID NO:146, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(j) an LCDR1 having the amino acid sequence shown in SEQ ID NO:122, an LCDR2 having the amino acid sequence shown in SEQ ID NO:135, an LCDR3 having the amino acid sequence shown in SEQ ID NO:147, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:109;
(k) an LCDR1 having the amino acid sequence shown in SEQ ID NO:123, an LCDR2 having the amino acid sequence shown in SEQ ID NO:136, an LCDR3 having the amino acid sequence shown in SEQ ID NO:148, an HCDR1 having the amino acid sequence shown in SEQ ID NO:92, an HCDR2 having the amino acid sequence shown in SEQ ID NO:100, and an HCDR3 having the amino acid sequence shown in SEQ ID NO:110;
(l) an LCDR1 having the amino acid sequence shown in SEQ ID NO:124, an LCDR2 having the amino acid sequence shown in SEQ ID NO:137, an LCDR3 having the amino acid sequence shown in SEQ ID NO:149, an HCDR1 having the amino acid sequence shown in SEQ ID NO:93, an HCDR2 having the amino acid sequence shown in SEQ ID NO:101, and an HCDR3 having the amino acid sequence shown in SEQ ID NO:111; and
(m) an LCDR1 having the amino acid sequence shown in SEQ ID NO:125, an LCDR2 having the amino acid sequence shown in SEQ ID NO:137, an LCDR3 having the amino acid sequence shown in SEQ ID NO:150, an HCDR1 having the amino acid sequence shown in SEQ ID NO:94, an HCDR2 having the amino acid sequence shown in SEQ ID NO:102 and an HCDR3 having the amino acid sequence shown in SEQ ID NO:112.

In some embodiments, the binding domain that specifically binds to CD7 comprises a light chain variable region and a heavy chain variable region selected from the group consisting of:
(a) a light chain variable region comprising the amino acid sequence shown in SEQ ID NO:35 and a heavy chain variable region comprising the amino acid sequence shown in SEQ ID NO:36;
(b) a light chain variable region comprising the amino acid sequence shown in SEQ ID NO:40 and a heavy chain variable region comprising the amino acid sequence shown in SEQ ID NO:41;
(c) a light chain variable region comprising the amino acid sequence shown in SEQ ID NO:44 and a heavy chain variable region comprising the amino acid sequence shown in SEQ ID NO:45;
(d) a light chain variable region comprising the amino acid sequence shown in SEQ ID NO:48 and a heavy chain variable region comprising the amino acid sequence shown in SEQ ID NO:49;
(e) a light chain variable region comprising the amino acid sequence shown in SEQ ID NO:52 and a heavy chain variable region comprising the amino acid sequence shown in SEQ ID NO:53;
(f) a light chain variable region comprising the amino acid sequence shown in SEQ ID NO:56 and a heavy chain variable region comprising the amino acid sequence shown in SEQ ID NO:57;
(g) a light chain variable region comprising the amino acid sequence shown in SEQ ID NO:60 and a heavy chain variable region comprising the amino acid sequence shown in SEQ ID NO:61;
(h) a light chain variable region comprising the amino acid sequence shown in SEQ ID NO:64 and a heavy chain variable region comprising the amino acid sequence shown in SEQ ID NO:65;
(i) a light chain variable region comprising the amino acid sequence shown in SEQ ID NO:68 and a heavy chain variable region comprising the amino acid sequence shown in SEQ ID NO:69;
(j) a light chain variable region comprising the amino acid sequence shown in SEQ ID NO:72 and a heavy chain variable region comprising the amino acid sequence shown in SEQ ID NO:73;
(k) a light chain variable region comprising the amino acid sequence shown in SEQ ID NO:76 and a heavy chain variable region comprising the amino acid sequence shown in SEQ ID NO:77;
(l) a light chain variable region comprising the amino acid sequence shown in SEQ ID NO:80 and a heavy chain variable region comprising the amino acid sequence shown in SEQ ID NO:81; and
(m) a light chain variable region comprising the amino acid sequence shown in SEQ ID NO:84 and a heavy chain variable region comprising the amino acid sequence shown in SEQ ID NO:85.

In some embodiments, the binding domain that specifically binds to CD7 is an scFv.

In some embodiments, the transmembrane domain is a CD8 transmembrane domain.

In some embodiments, the intracellular signaling domain comprises a 4-1BB co-stimulatory domain and a CD3 zeta intracellular domain.

In some embodiments, the chimeric antigen receptor further comprises a hinge region between the binding domain and the transmembrane domain.

In some embodiments, the hinge region is a CD8 hinge region.

In some embodiments, the chimeric antigen receptor targeting CD7 comprises an amino acid sequence selected from SEQ ID NO:37, 42, 46, 50, 54, 58, 62, 66, 70, 74, 78, 82, 86.

In some embodiments, the chimeric antigen receptor targeting CD7 further comprises a signal peptide.

In some embodiments, the signal peptide comprised by the chimeric antigen receptor targeting CD7 is a CD8 signal peptide.

In some embodiments, step (a) comprises knocking out endogenous TRAC, B2M and CD7 genes in the T cells.

In some embodiments, step (a) comprises introducing into the T cells a CRISPR-Cas protein and gRNAs targeting the endogenous TRAC, B2M, and CD7 genes in the T cells, to induce cleavage in the endogenous TRAC, B2M, and CD7 genes of the T cells and disrupt their expression.

In some embodiments, the gRNA targeting the TRAC gene comprises a guide sequence selected from SEQ ID NO:23 and SEQ ID NO:24.

In some embodiments, the gRNA targeting the B2M gene comprises a guide sequence selected from SEQ ID NO:15 and SEQ ID NO:22.

In some embodiments, the gRNA targeting the CD7 gene comprises a guide sequence as shown in SEQ ID NO:25.

In some embodiments, the gRNAs targeting TRAC, B2M, and CD7 genes respectively comprise guide sequences selected from the group consisting of:

The gRNA targeting the TRAC gene comprises a guide sequence as shown in SEQ ID NO:23, the gRNA targeting the B2M gene comprises a guide sequence as shown in SEQ ID NO:15, and the gRNA targeting the CD7 gene comprises a guide sequence as shown in SEQ ID NO:25; and

The gRNA targeting the TRAC gene comprises a guide sequence as shown in SEQ ID NO:24, the gRNA targeting the B2M gene comprises a guide sequence as shown in SEQ ID NO:15, and the gRNA targeting the CD7 gene comprises a guide sequence as shown in SEQ ID NO:25.

In some embodiments, the polynucleotide sequence comprises homology arms flanking the nucleic acid sequence encoding the chimeric antigen receptor, which are homologous to the upstream and downstream sequences of the TRAC gene cleavage site, respectively, so that the nucleic acid sequence encoding the chimeric antigen receptor is inserted into the TRAC locus by homologous recombination.

In some embodiments, the nucleic acid sequence encoding the chimeric antigen receptor is inserted into the TRAC locus at a position operably linked to the TRAC endogenous promoter.

In some embodiments, the polynucleotide sequence comprises a PGK promoter operably linked to a nucleic acid sequence encoding a chimeric antigen receptor.

In some embodiments, the polynucleotide sequence is contained in a viral vector.

In some embodiments, the viral vector is an AAV vector.

In some embodiments, the method further comprises:
(c) introducing a polynucleotide sequence comprising a nucleic acid sequence encoding a fusion protein of a lymphocyte-antigen presenting cell co-stimulator into the T cell,
The fusion protein of lymphocyte-antigen presenting cell co-stimulator comprises a first domain for activating antigen presenting cells (APC) and a second domain for activating immune effector cells, wherein
The first domain comprises: (i) a ligand that binds to an activating receptor of the APC, or a receptor binding fragment thereof, or (ii) an antibody that binds to an activating receptor of the APC, or an antigen-binding fragment thereof; and
The second domain comprises: (i) a co-stimulatory receptor of the immune effector cell, or a functional fragment thereof, (ii) a co-stimulatory ligand of the immune effector cell, or a receptor binding fragment thereof, or (iii) an antibody that binds to the co-stimulatory receptor of the immune effector cell, or an antigen-binding fragment thereof.

In some embodiments, the activating receptor of the APC is CD40.

In some embodiments, the first domain is an anti-CD40 antibody or an antigen-binding fragment thereof.

In some embodiments, the first domain is an scFv.

In some embodiments, the immune effector cells are T cells.

In some embodiments, the second domain comprises the intracellular domain of the co-stimulatory receptor.

In some embodiments, the co-stimulatory receptor is CD28.

In some embodiments, the second domain further comprises a transmembrane domain of the co-stimulatory receptor.

In some embodiments, the first domain and the second domain are connected via a CD28 hinge region.

In some embodiments, the lymphocyte-antigen presenting cell co-stimulatory fusion protein comprises the amino acid sequence shown in SEQ ID NO:161.

In some embodiments, the lymphocyte-antigen presenting cell co-stimulatory fusion protein further comprises a signal peptide.

In some embodiments, the signal peptide contained in the lymphocyte-antigen presenting cell co-stimulatory fusion protein is a CD8 signal peptide.

In some embodiments, the nucleic acid sequence encoding the lymphocyte-antigen presenting cell co-stimulatory fusion protein and the nucleic acid sequence encoding the chimeric antigen receptor are in the same expression frame or in separate expression frames.

Another aspect of the present invention also provides modified T cells produced by the above method.

Another aspect of the present invention provides a pharmaceutical composition, which includes any of the aforementioned modified T cells, or modified T cells produced according to any of the aforementioned methods, and a pharmaceutically acceptable carrier.

Another aspect of the present invention provides a method for treating a disease associated with CD7 expression, including administering a therapeutically effective amount of any one of the aforementioned modified T cells or the aforementioned pharmaceutical composition to a subject in need thereof.

Another aspect of the present invention provides use of any of the aforementioned modified T cells, or modified T cells produced according to any of the aforementioned methods, in the preparation of a medicament for treating a disease associated with CD7 expression.

### BRIEF DESCRIPTION OF THE DRAWINGS

FIG. 1. Plasmid map of pDA-T7-B2M gRNA. FIG. 1A represents the plasmid map containing B2M gRNA-1, and FIG. 1B shows the nucleotide sequences of the promoter, g RNA guide sequence, gRNA scaffold sequence, and termination signal contained in the plasmid.

FIG. 2. Flow cytometry analysis screening results from 22 candidate gRNAs for CRISPR/Cas9/B2M gRNA gene knockout in T cells.

FIG. 3. Flow cytometry analysis and result summary of simultaneous knockout of TRAC/B2M/CD7 genes in T cells using CRISPR/Cas9 with different gRNA combinations. The results show that the gene knockout effect of TRAC gRNA3.4 is better than that of TRAC gRNA19.6, and the gene knockout effect of B2M gRNA-11 is better than that of B2M gRNA-22.

FIG. 4. Readout of anti-human CD7-Fc monoclonal phage ELISA in three 96-well plates.

FIG. 5. Schematic diagram of the structures of three AAV vectors: R196H1A, R3H1A and R3PGK.

FIG. 6. Technical strategy for preparing TRAC/B2M/CD7 triKO CD7 CAR-T by simultaneous knockout of three genes (TRAC/B2M/CD7) and infection with three AAVs: R196H1A, R3H1A and R3PGK.

FIG. 7. Flow cytometry analysis of the efficiency of TCR and B2M knockout after simultaneous knockout of TRAC/B2M/CD7 genes in T cells and infection with three AAVs, R196H1A, R3H1A and R3PGK, for 4 days.

FIG. 8. Flow cytometry analysis of the efficiency of TCR and CD7 knockout after simultaneous knockout of TRAC/B2M/CD7 genes in T cells and infection with three AAVs, R196H1A, R3H1A and R3PGK, for 4 days.

FIG. 9. Flow cytometry analysis of CD7H1 CAR expression in T cells using CD7-Fc recombinant protein, performed 4 days after simultaneous knockout of TRAC/B2M/CD7 genes and infection with three AAVs, R196H1A, R3H1A and R3PGK.

FIG. 10. Flow cytometry analysis of LACO expression in T cells using CD40-Fc recombinant protein, performed 4 days after simultaneous knockout of TRAC/B2M/CD7 genes and infection with three AAVs, R196H1A, R3H1A and R3PGK.

FIG. 11. Flow cytometry analysis of the efficiency of TCR and B2M knockout after simultaneous knockout of TRAC/B2M/CD7 genes in T cells and infection with three AAVs, R196H1A, R3H1A and R3PGK, for 8 days.

FIG. 12. Flow cytometry analysis of the efficiency of TCR and CD7 knockout after simultaneous knockout of TRAC/B2M/CD7 genes in T cells and infection with three AAVs, R196H1A, R3H1A and R3PGK, for 8 days.

FIG. 13. Flow cytometry analysis of CD7H1 CAR expression in T cells using CD7-Fc recombinant protein, performed 8 days after simultaneous knockout of TRAC/B2M/CD7 genes and infection with three AAVs, R196H1A, R3H1A and R3PGK.

FIG. 14. Flow cytometry analysis of LACO expression in T cells using CD40-Fc recombinant protein, performed 8 days after simultaneous knockout of TRAC/B2M/CD7 genes and infection with three AAVs, R196H1A, R3H1A and R3PGK.

FIG. 15. Statistical analysis of the number of cells proliferating and expanding during in vitro culture of triKO CD7 CAR-T cells and control T cells prepared by infection with three AAV viruses R196H1A, R3H1A and R3PGK at different MOIs.

FIG. 16. The killing effect of triKO CD7 CAR-T cells and control T cells prepared by infecting three AAV viruses R196H1A, R3H1A and R3PGK with different MOIs and co-incubating with CD7-positive CCRF-CEM tumor target cell line at E:T=2:1 in vitro was analyzed by inCucyte.

FIG. 17. The killing effect of triKO CD7 CAR-T cells and control T cells prepared by infecting three AAV viruses R196H1A, R3H1A and R3PGK with different MOIs and co-incubating with CD7-positive CCRF-CEM tumor target cell line at E:T=0.5:1 in vitro was analyzed by inCucyte.

FIG. 18. The killing effect of triKO CD7 CAR-T cells and control T cells prepared by infecting three AAV viruses R196H1A, R3H1A and R3PGK with different MOIs and co-incubated with CD7-positive CCRF-CEM tumor target cell line at E:T=0.2:1 in vitro was analyzed by inCucyte.

### DETAILED DESCRIPTION

The present invention prepares a universal CD7 CAR-T by collecting T cells from healthy donors for gene editing to avoid allogeneic rejection and fratricide. The present invention uses CRISPR/Cas9 technology to simultaneously knock out the TCR, B2M (Beta-2 microglobulin) and CD7 genes of T cells, and while knocking out the genes, adds AAV of CD7 CAR and LACO co-stimulatory molecules as donor DNA fragments encoding CD7H1.BBZ.A40C2828, integrates them into the genomic locus of TRAC, and uses the endogenous promoter of TRAC or the exogenous PGK promoter to drive the expression of CD7 CAR and LACO co-stimulatory molecules, thereby preparing a universal CAR-T targeting CD7 molecules.

The experimental results show that CRISPR/Cas9 technology successfully and efficiently knocks out the TCR, B2M and CD7 genes of T cells, and AAV, as a donor DNA fragment-mediated site-directed integration, also achieved high efficiency. CD7 CAR and LACO molecules were successfully expressed on the surface of T cell membranes. The results of in vitro killing experiments co-cultured with CD7-positive tumor target cell lines showed that the triKO CD7 CAR-T cells with TCR/B2M/CD7 triple knockout prepared in this way had a very strong killing effect.

Unless otherwise indicated, the terms used in the present invention have the meanings generally understood in the art and can be understood by referring to standard textbooks, reference books, and documents known to those skilled in the art. All publications mentioned herein are incorporated herein by reference in their entirety.

It should be understood that the specific methods and materials described in the embodiments of the present invention are only for the purpose of describing specific embodiments and are not restrictive. Any methods and materials similar to or equivalent to the methods and materials described herein can be used in the practice or testing of the present invention. The explanation of the relevant theory or mechanism in the present invention is only used to help understand the invention and should not be regarded as a limitation on the scheme protected by the present invention.

The term “comprising” as used herein, or other term forms with similar meanings such as “including”, “containing” or “having”, etc., should be understood to include the listed elements, but not to exclude the presence of other elements. These terms also include the situation of consisting of only the listed elements. The term “consisting of . . . ” means consisting of only the listed elements. The term “consisting essentially of . . . ” means that elements that have no significant effect on the scheme involved are not excluded.

Unless otherwise specifically stated, “a”, “an” or “the” herein include both singular and plural forms. The meaning of the term “at least one” or “at least one type” or other similar expressions shall be construed to mean “one or more” or “one type or more type”. The numerical ranges described herein, such as temperature ranges, time ranges, composition or concentration ranges, or other numerical ranges, include the end values, all intermediate ranges, and sub-ranges (e.g., the range between a certain intermediate value and a certain end value) within the range. Furthermore, any intermediate ranges, sub-ranges, and all individual values described in the numerical range can be excluded from the numerical range.

The terms “about” or “approximately” used herein mean a range of +10% of the numerical value.

The “and/or” described herein should be understood as any one element or any combination of several elements connected by this term.

In the present invention, unless otherwise indicated, nucleic acid sequences are generally described from the 5′ end to the 3′ end, and amino acid sequences are generally described from the N-terminus to the C-terminus.

The present invention provides a CAR-T cell targeting CD7, which expresses a chimeric antigen receptor targeting CD7. The endogenous TRAC and B2M genes of the CAR-T cell are suppressed, and the CAR-T cell will not cause graft-versus-host disease (GVHD) when it is re-infused into an allogeneic subject; the endogenous CD7 gene of the CAR-T cell is suppressed, so that the CAR-T cell will not have a killing effect on itself.

“T cell” herein, i.e. T lymphocyte, may include thymocytes, natural T lymphocytes, immature T lymphocytes, mature T lymphocytes, resting T lymphocytes or activated T lymphocytes.

The T cell can be a helper T cell (Th), a cytotoxic T cell (CTL), a memory T cell, a regulatory T cell, a natural killer T cell, a CD4+/CD8+T cell, a CD4−/CD8−T cell or any other T lymphocyte subtype. In some cases, the modified T cell is a human T cell. Before amplification and genetic modification of the T cell of the present application, a cell source can be obtained from a subject, such as a patient, by various non-limiting methods. T cells can be obtained from many non-limiting sources, including peripheral blood mononuclear cells, bone marrow, lymph node tissue, cord blood, thymus tissue, tissue of infection site, ascites, pleural effusion, spleen tissue and tumor. In some cases, any number of T cell lines available and known to those skilled in the art can be used. In other cases, the T cells can be derived from healthy donors, from patients diagnosed with cancer, or obtained from patients diagnosed with infection. In other cases, the T cells are cell population, such as part of a mixed population of cells with different phenotypic properties. In some embodiments, the T cells are autologous T cells for the subject in need of treatment, and in other embodiments, the T cells are allogeneic T cells for the subject in need of treatment.

The term “endogenous” refers to a nucleotide sequence that occurs naturally in a cell prior to being modified as described herein.

The TRAC gene encodes the TCR alpha chain, one of the components of the TCR complex.

Disruption of the TRAC gene results in loss of TCR function and renders engineered T cells non-alloreactive and suitable for allogeneic transplantation, thereby minimizing the risk of graft-versus-host disease.

The B2M gene encodes beta-2 microglobulin, the light chain of the MHC class I molecule. The β2M gene encodes the common (invariant) component of the major histocompatibility complex (MHC) I complex. Disruption of the β2M gene prevents host anti-therapeutic allogeneic T cell responses. Knocking out the TRAC gene and the β2M gene will allow the generation of allogeneic T cells for cell therapy.

The term “CD7” refers to cluster of differentiation 7 which is well known in the art.

The term “suppress” refers to reducing or eliminating the expression of a gene. The suppression will result in a 10%, 20%, 30%, 40%, 50%, 60%, 70%, 80%, 90% or 100% reduction in the level of the gene product. Methods for measuring the level of the gene product are known by those skilled in the art, and can be measured before and after adopting a means of suppressing gene expression to assess the effect of gene suppression.

In the present invention, gene expression can be reduced (i.e., knocked down) by means such as antisense RNA, siRNA, shRNA, etc.; gene expression can also be eliminated (i.e., knocked out) by gene editing techniques such as zinc finger nucleases (ZFNs), transcription activator-like effector nucleases (TALENs) or CRISPR-Cas systems and methods thereof.

The term “knockdown” as used herein refers to a detectable decrease (e.g., detected by antibodies or flow cytometry) in the expression of a target gene or corresponding protein in a cell after a measure to suppress gene expression is taken, compared to the expression of the target gene or corresponding protein in the cell before the measure to suppress gene expression is taken.

The term “knockout” may also be referred to herein as “disruption” or “inactivation”. A disrupted gene does not express (e.g., not expressed on the cell surface) a detectable level (e.g., detected by antibodies or flow cytometry) of a protein encoded by the gene. In some embodiments, the term “knockout” includes deleting all or part of a target gene sequence.

The CRISPR-Cas system is a naturally occurring defense mechanism in prokaryotes that has been used as an RNA-guided DNA targeting platform for gene editing. The CRISPR-Cas system includes a Cas nuclease and two non-coding RNAs (crRNA and tracrRNA), which can form a CRISPR-Cas complex that is guided to the target sequence by hybridization of a guide sequence of about 20 nt contained in the crRNA with a target sequence in the target gene, and then the Cas protein cleaves the DNA at a specific position near the PAM site, resulting in a double-strand break (DSB). Many types and species of CRISPR-Cas systems have been described (see, for example, Koonin et al., 2017, Curr. Opin. Microbiol., 37:67-78).

In the present invention, the Cas proteins that can be used include, but are not limited to Cas9, Cas12, etc., and their orthologs or variants. Cas9 proteins include but are not limited to Cas9 proteins from Streptococcus pyogenes (SpCas9) or Cas9 proteins from Staphylococcus aureus (SaCas9).

The term “gRNA” refers to a guide RNA capable of forming a complex with Cas protein and directing the Cas protein to the target sequence for cleavage. The gRNA may include crRNA and tracrRNA. The crRNA contains a guide sequence that can be paired with the target sequence through base complementation. The gRNA can be a bimolecular gRNA or a single molecule gRNA (sgRNA).

The Cas protein can be introduced into the cell in the form of a protein or in the form of its encoding nucleic acid sequence (e.g., mRNA or cDNA). The nucleic acid encoding the Cas protein can be contained in a plasmid or a viral vector and introduced into the cell, for example, by transfection. The Cas protein or the nucleic acid encoding the Cas protein can be directly delivered to the cell by electroporation, liposomes, microinjection, etc.

gRNA can be introduced into cells by any method suitable for introducing RNA into cells. For example, gRNA can be introduced into cells in the form of isolated RNA. Any in vitro transcription system known in the art can be used to prepare isolated gRNA by in vitro transcription, and then gRNA is introduced into cells by electroporation. gRNA can also be introduced into cells by a vector comprising a sequence encoding gRNA and a promoter. The vector can be a viral vector or a plasmid, and the method of introducing cells can be transfection.

The term “target gene” as used herein refers to a gene whose gene expression is to be reduced or eliminated.

The term “target sequence” refers to a nucleotide sequence contained in the target gene, which is complementary to the guide sequence contained in the gRNA through base pairing, thereby guiding the CRISPR-Cas complex to cleave the target gene. A target gene can contain multiple target sequences. By designing gRNAs that complementarily pair with different target sequences, the CRISPR-Cas system can cleave at different locations of the same target gene.

The term “guide sequence” refers to a sequence that is complementary to a target sequence in a target gene through base pairing, which is contained in crRNA. The CRISPR-Cas complex is located at the target sequence for cleavage by hybridization of the guide sequence and the target sequence.

The degree of complementarity between the guide sequence and the target sequence in the target gene can be at least 70%, about 75%, about 80%, about 85%, about 90%, about 95% or 100%. In some embodiments, the guide sequence is 100% complementary to the target sequence in the target gene. In some embodiments, the guide sequence may include up to 5, up to 4, up to 3, up to 2 or up to 1 mismatches with the target sequence in the target gene. In some embodiments, the length of the guide sequence and/or the target sequence can be about 15 to about 25 nucleotides. In some embodiments, the length of the guide sequence and/or the target sequence can be about 15, about 16, about 17, about 18, about 19, about 20, about 21, about 22, about 23, about 24 or about 25 nucleotides. In some embodiments, the guide sequence included in the gRNA for targeting the B2M gene can be selected from SEQ ID NO:1-22. In some embodiments, the guide sequence included in the gRNA for targeting the B2M gene is SEQ ID NO:15 or SEQ ID NO:21. The guide sequence contained in the gRNA for targeting the TRAC gene can be selected from SEQ ID NO:23 or SEQ ID NO:24. The guide sequence contained in the gRNA for targeting the CD7 gene can be SEQ ID NO:25.

The three endogenous genes TRAC, B2M and CD7 in the modified T cells of the present invention can be knocked out simultaneously or separately. For example, TRAC, B2M and CD7 can be knocked out using one or more different CRISPR-Cas systems, wherein the one or more different CRISPR-Cas systems respectively contain different Cas proteins and corresponding gRNAs. One of the CRISPR-Cas systems can be used to knock out one, two or three of TRAC, B2M and CD7, and other CRISPR-Cas systems can be used to knock out the remaining genes. The gRNAs targeting different genes can be located on the same or different vectors and can be operably connected to the same or different promoters, respectively.

In some embodiments, three endogenous genes TRAC, B2M and CD7 are knocked out simultaneously. For example, gRNA targeting TRAC, gRNA targeting B2M and gRNA targeting CD7 are used together with Cas protein to make the CRISPR-Cas system cleave three endogenous genes TRAC, B2M and CD7 simultaneously.

The DSB caused by CRISPR-Cas system cleaving at the target site can be repaired by non-homologous end joining (NHEJ) or homology-directed recombination (HDR), resulting in insertions or deletions in the target gene, thereby achieving disruption and knockout of the target gene.

NHEJ is highly active in most cell types, including non-dividing cells, and usually results in the removal or addition of one to several hundred nucleotides at the site of DSB. The resulting insertions and deletions (indels) can disrupt the coding or non-coding regions of the gene (depending on the design of the gRNA). Alternatively, HDR uses a donor repair template to repair DSB with high fidelity. The donor repair template contains homology arms that are homologous to the sequences on both sides of the DSB site on the target sequence, and the sequence between the homology arms is inserted into the target sequence by homologous recombination. HDR is only effective in dividing cells.

In some embodiments, knockout of endogenous TRAC in T cells by the CRISPR-Cas system can be repaired by NHEJ or HDR. In some embodiments, knockout of endogenous B2M in T cells by the CRISPR-Cas system can be repaired by NHEJ or HDR. In some embodiments, knockout of endogenous CD7 in T cells by the CRISPR-Cas system can be repaired by NHEJ or HDR.

The donor repair template comprises homology arms homologous to the sequences on both sides of the DSB site on the target sequence. The length of the homology arms can be about 100 bp, about 200 bp, about 300 bp, about 400 bp, about 500 bp, about 600 bp, about 700 bp, about 800 bp, about 900 bp, about 1000 bp or longer.

In some embodiments, the knockout of endogenous TRAC in T cells is repaired by HDR through the CRISPR-Cas system. The donor repair template used has homology arms homologous to the sequences on both sides of the DSB site of endogenous TRAC gene, B2M gene or CD7 gene in T cells. In some embodiments, the sequence of the 5′ homology arm of the donor repair template is as shown in SEQ ID NO:172, and the sequence of the 3′ homology arm is as shown in SEQ ID NO:173. In some embodiments, the sequence of the 5′ homology arm of the donor repair template is as shown in SEQ ID NO:174, and the sequence of the 3′ homology arm is as shown in SEQ ID NO:175.

The donor repair template can be introduced into T cells in the form of naked nucleic acid or can be contained in a vector. In some embodiments, the donor repair template is introduced into T cells by a vector. The term “vector” is used herein to refer to a nucleic acid molecule that can transfer or transport another nucleic acid molecule. The transferred nucleic acid is generally connected to a carrier nucleic acid molecule, such as inserted into a carrier nucleic acid molecule. Illustrative examples of vectors include, but are not limited to, plasmid vectors, viral vectors, autonomous replication sequences, and transposable elements. Examples of viral vectors include, but are not limited to, adeno-associated viruses (AAV), adenoviruses, retroviruses (including lentiviruses), herpes viruses (such as herpes simplex viruses), poxviruses, baculoviruses, papillomavirus vectors, etc. In some embodiments, the vector is an AAV vector.

The term “CAR-T cells” refers to a T cell expressing a chimeric antigen receptor (CAR), which enables specific recognition and killing of cells expressing a target antigen recognized by the chimeric antigen receptor.

The term “chimeric antigen receptor” or “CAR” refers to an artificial T cell surface receptor that is modified to be expressed on immune effector cells and specifically binds to an antigen.

A chimeric antigen receptor generally comprises an extracellular binding domain, a transmembrane domain, and an intracellular signaling domain. In some embodiments, a chimeric antigen receptor may further comprise a hinge region between the binding domain and the transmembrane domain.

The term “binding domain” refers to a domain capable of specific binding to a target antigen.

The binding domain may comprise an antibody or a ligand or a fragment thereof that can specifically bind to a target antigen.

The term “antibody” refers to a polypeptide molecule that capable of specifically recognizing and/or neutralizing a target antigen. For example, an antibody may comprise an immunoglobulin comprising at least two heavy (H) chains and two light (L) chains interconnected by disulfide bonds, and includes any molecule comprising an antigen-binding portion thereof. The term “antibody” includes monoclonal antibodies, antigen-binding or antibody derivatives, including but not limited to human antibodies (fully human antibodies), humanized antibodies, chimeric antibodies, single-chain antibodies (e.g., scFv), and antibody fragments that bind to antigens (e.g., Fab, Fab′ and (Fab)2 fragments). The term “antibody” also includes all recombinant forms of antibodies, such as antibodies expressed in prokaryotic cells, unglycosylated antibodies, and any antibody fragments and derivatives thereof that bind to antigens described herein. Each heavy chain is composed of a heavy chain variable region (VH) and a heavy chain constant region. Each light chain can be composed of a light chain variable region (VL) and a light chain constant region. The VH and VL regions can be further divided into hypervariable regions termed complementary determining regions (CDRs), which are interspersed in more conserved regions known as framework regions (FRs). Each VH and VL is composed of three CDRs and four FRs, which can be arranged in the following order from the amino terminus to the carboxyl terminus: FR1, CDR1, FR2, CDR2, FR3, CDR3, and FR4. In this article, the three heavy chain complementary determining regions are referred to as HCDR1, HCDR2, and HCDR3, respectively, and the four heavy chain framework regions are referred to as HFR1, HFR2, HFR3, and HFR4, respectively; the three light chain complementary determining regions are referred to as LCDR1, LCDR2, and LCDR3, respectively, and the four light chain framework regions are referred to as LFR1, LFR2, LFR3, and LFR4, respectively. The variable regions (VH and VL) of the heavy and light chains form antigen binding sites, respectively. The constant regions of the antibodies may mediate the binding of the immunoglobulin to host tissues or factors, including various cells of the immune system (eg, effector cells) and the first component (Clq) of the classical complement system.

As used herein, the term “complementarity determining region” or “CDR” refers to the amino acid residues in the variable region of an antibody that are responsible for antigen binding.

The precise boundaries of a CDR can be defined according to various numbering systems known in the art, such as the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al., (1989) Nature 342:878-883) or the IMGT numbering system (Lefranc et al., Dev. Comparat. Immunol. 27:55-77, 2003). For a given antibody, one skilled in the art will readily identify the CDRs defined according to each numbering system. Moreover, the correspondence between different numbering systems is well known to those skilled in the art (e.g., see Lefranc et al., Dev. Comparat. Immunol. 27:55-77, 2003). The antibodies of the present invention can use any of these numbering systems to define CDRs, although it is preferred to use the Clothia numbering system to define CDRs.

The term “antigen-binding fragment” refers to one or more fragments of a full-length antibody that retain the ability to specifically bind to a given antigen. Antigen-binding fragments can be obtained by any suitable technique, such as proteolytic digestion of intact antibodies or recombinant DNA technology. Examples of the term “antigen-binding fragment” include (i) Fab fragment, a monovalent fragment consisting of VL, VH, CL and CH1 domains; (ii) Fab′ fragment, which is essentially Fab with a portion of the hinge region; (iii) F(ab′)₂ fragment, a bivalent fragment comprising two Fab fragments linked by a disulfide bond at the hinge region; (iv) Fd fragment consisting of VH and CH1 domains; (v) Fv fragment consisting of the VL and VH domains of a single arm of an antibody; (vi) single-chain Fv fragment (scFv, also named as single-chain antibody), in which the VL and VH regions are paired to form a single protein chain of a monovalent molecule); (vii) disulfide-stabilized Fv fragment (dsFv), an Fv with an artificially engineered intermolecular disulfide bond that stabilizes the VH-VL pair; (viii) single-domain antibodies (sdAbs) consisting only of the heavy chain variable region, etc.

In some embodiments, the binding domain comprised by the CAR of the present invention is an anti-CD7 antibody or an antigen-binding fragment thereof.

In some embodiments, the anti-CD7 antibody comprises a light chain variable region and a heavy chain variable region, the light chain variable region comprising LCDR1, LCDR2 and LCDR3, and the heavy chain variable region comprising HCDR1, HCDR2 and HCDR3; wherein, LCDR1 has an amino acid sequence selected from SEQ ID NOs: 113-125; LCDR2 has an amino acid sequence selected from SEQ ID NOs: 126-137; LCDR3 has an amino acid sequence selected from SEQ ID NOs: 138-150; HCDR1 has an amino acid sequence selected from SEQ ID NOs: 87-94; HCDR2 has an amino acid sequence selected from SEQ ID NOs: 95-102; and HCDR3 has an amino acid sequence selected from SEQ ID NOs: 103-112.

In some embodiments, the light chain variable region and the heavy chain variable region of the anti-CD7 antibody comprise LCDR1, LCDR2, LCDR3, HCDR1, HCDR2, and HCDR3 selected from the group consisting of:
(a) an LCDR1 having the amino acid sequence shown in SEQ ID NO:113, an LCDR2 having the amino acid sequence shown in SEQ ID NO:126, an LCDR3 having the amino acid sequence shown in SEQ ID NO:138, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(b) an LCDR1 having the amino acid sequence shown in SEQ ID NO:114, an LCDR2 having the amino acid sequence shown in SEQ ID NO:127, an LCDR3 having the amino acid sequence shown in SEQ ID NO:139, an HCDR1 having the amino acid sequence shown in SEQ ID NO:88, an HCDR2 having the amino acid sequence shown in SEQ ID NO:96, and an HCDR3 having the amino acid sequence shown in SEQ ID NO:104;
(c) an LCDR1 having the amino acid sequence shown in SEQ ID NO:115, an LCDR2 having the amino acid sequence shown in SEQ ID NO:128, an LCDR3 having the amino acid sequence shown in SEQ ID NO:140, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:105;
(d) an LCDR1 having the amino acid sequence shown in SEQ ID NO:116, an LCDR2 having the amino acid sequence shown in SEQ ID NO:129, an LCDR3 having the amino acid sequence shown in SEQ ID NO:141, an HCDR1 having the amino acid sequence shown in SEQ ID NO:89, an HCDR2 having the amino acid sequence shown in SEQ ID NO:97, and an HCDR3 having the amino acid sequence shown in SEQ ID NO:106;
(e) an LCDR1 having the amino acid sequence shown in SEQ ID NO:117, an LCDR2 having the amino acid sequence shown in SEQ ID NO:130, an LCDR3 having the amino acid sequence shown in SEQ ID NO:142, an HCDR1 having the amino acid sequence shown in SEQ ID NO:90, an HCDR2 having the amino acid sequence shown in SEQ ID NO:98, and an HCDR3 having the amino acid sequence shown in SEQ ID NO:107;
(f) an LCDR1 having the amino acid sequence shown in SEQ ID NO:118, an LCDR2 having the amino acid sequence shown in SEQ ID NO:131, an LCDR3 having the amino acid sequence shown in SEQ ID NO:143, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(g) an LCDR1 having the amino acid sequence shown in SEQ ID NO:119, an LCDR2 having the amino acid sequence shown in SEQ ID NO:132, an LCDR3 having the amino acid sequence shown in SEQ ID NO:144, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(h) an LCDR1 having the amino acid sequence shown in SEQ ID NO:120, an LCDR2 having the amino acid sequence shown in SEQ ID NO:133, an LCDR3 having the amino acid sequence shown in SEQ ID NO:145, an HCDR1 having the amino acid sequence shown in SEQ ID NO:91, an HCDR2 having the amino acid sequence shown in SEQ ID NO:99, and an HCDR3 having the amino acid sequence shown in SEQ ID NO:108;
(i) an LCDR1 having the amino acid sequence shown in SEQ ID NO:121, an LCDR2 having the amino acid sequence shown in SEQ ID NO:134, an LCDR3 having the amino acid sequence shown in SEQ ID NO:146, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(j) an LCDR1 having the amino acid sequence shown in SEQ ID NO:122, an LCDR2 having the amino acid sequence shown in SEQ ID NO:135, an LCDR3 having the amino acid sequence shown in SEQ ID NO:147, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:109;
(k) an LCDR1 having the amino acid sequence shown in SEQ ID NO:123, an LCDR2 having the amino acid sequence shown in SEQ ID NO:136, an LCDR3 having the amino acid sequence shown in SEQ ID NO:148, an HCDR1 having the amino acid sequence shown in SEQ ID NO:92, an HCDR2 having the amino acid sequence shown in SEQ ID NO:100, and an HCDR3 having the amino acid sequence shown in SEQ ID NO:110;
(l) an LCDR1 having the amino acid sequence shown in SEQ ID NO:124, an LCDR2 having the amino acid sequence shown in SEQ ID NO:137, an LCDR3 having the amino acid sequence shown in SEQ ID NO:149, an HCDR1 having the amino acid sequence shown in SEQ ID NO:93, an HCDR2 having the amino acid sequence shown in SEQ ID NO:101, and an HCDR3 having the amino acid sequence shown in SEQ ID NO:111; and
(m) an LCDR1 having the amino acid sequence shown in SEQ ID NO:125, an LCDR2 having the amino acid sequence shown in SEQ ID NO:137, an LCDR3 having the amino acid sequence shown in SEQ ID NO:150, an HCDR1 having the amino acid sequence shown in SEQ ID NO:94, an HCDR2 having the amino acid sequence shown in SEQ ID NO:102 and an HCDR3 having the amino acid sequence shown in SEQ ID NO:112.

In some embodiments, the anti-CD7 antibody comprises a light chain variable region and a heavy chain variable region selected from the group consisting of:
(a) a light chain variable region comprising the amino acid sequence shown in SEQ ID NO:35 and a heavy chain variable region comprising the amino acid sequence shown in SEQ ID NO:36;
(b) a light chain variable region comprising the amino acid sequence shown in SEQ ID NO:40 and a heavy chain variable region comprising the amino acid sequence shown in SEQ ID NO:41;
(c) a light chain variable region comprising the amino acid sequence shown in SEQ ID NO:44 and a heavy chain variable region comprising the amino acid sequence shown in SEQ ID NO:45;
(d) a light chain variable region comprising the amino acid sequence shown in SEQ ID NO:48 and a heavy chain variable region comprising the amino acid sequence shown in SEQ ID NO:49;
(e) a light chain variable region comprising the amino acid sequence shown in SEQ ID NO:52 and a heavy chain variable region comprising the amino acid sequence shown in SEQ ID NO:53;
(f) a light chain variable region comprising the amino acid sequence shown in SEQ ID NO:56 and a heavy chain variable region comprising the amino acid sequence shown in SEQ ID NO:57;
(g) a light chain variable region comprising the amino acid sequence shown in SEQ ID NO:60 and a heavy chain variable region comprising the amino acid sequence shown in SEQ ID NO:61;
(h) a light chain variable region comprising the amino acid sequence shown in SEQ ID NO:64 and a heavy chain variable region comprising the amino acid sequence shown in SEQ ID NO:65;
(i) a light chain variable region comprising the amino acid sequence shown in SEQ ID NO:68 and a heavy chain variable region comprising the amino acid sequence shown in SEQ ID NO:69;
(j) a light chain variable region comprising the amino acid sequence shown in SEQ ID NO:72 and a heavy chain variable region comprising the amino acid sequence shown in SEQ ID NO:73;
(k) a light chain variable region comprising the amino acid sequence shown in SEQ ID NO:76 and a heavy chain variable region comprising the amino acid sequence shown in SEQ ID NO:77;
(l) a light chain variable region comprising the amino acid sequence shown in SEQ ID NO:80 and a heavy chain variable region comprising the amino acid sequence shown in SEQ ID NO:81; and
(m) a light chain variable region comprising the amino acid sequence shown in SEQ ID NO:84 and a heavy chain variable region comprising the amino acid sequence shown in SEQ ID NO:85.

In some embodiments, the binding domain in the CAR of the present invention is a single-chain antibody (scFv), which comprises an antibody light chain and an antibody heavy chain connected in tandem. In some embodiments, in the single-chain antibody, the C-terminus of the antibody light chain is connected to the N-terminus of the antibody heavy chain. In some embodiments, in the single-chain antibody, the C-terminus of the antibody heavy chain is connected to the N-terminus of the antibody light chain. In some embodiments, in the single-chain antibody, the light chain and the heavy chain are connected via a linker. In some embodiments, the linker between the antibody light chain and the heavy chain has a sequence as shown in SEQ ID NO:177 or SEQ ID NO:178.

In some embodiments, the anti-CD7 scFv comprises the amino acid sequence shown in SEQ ID NOs: 34, 39, 43, 47, 51, 55, 59, 63, 67, 71, 75, 79 or 83.

The term “transmembrane domain” refers to a domain in the CAR that spans the cell membrane and is connected to the intracellular signaling domain and plays a role in transmitting signals. Transmembrane domains from the following proteins can be used in CAR: α, β or ζ chain of T cell receptor, CD28, CD3e, CD45, CD4, CD5, CD8, CD9, CD16, CD22, CD33, CD37, CD64, CD80, CD86, CD134, CD137 or CD154. In some embodiments, the CAR of the present invention comprises a CD8 transmembrane domain. In some embodiments, the CD8 transmembrane domain comprises an amino acid sequence as shown in SEQ ID NO:155.

The term “signaling domain” is a part that conducts information about the binding of CAR to the target antigen to the interior of the immune effector cell to cause effector cell function, such as activation, cytokine production, proliferation, and cytotoxic activity, including the release of cytotoxic factors into target cells bound to CAR, or other cell responses caused by antigen binding to the extracellular CAR domain. The effector function of T cells can be, for example, cytolytic activity or help activity, including the secretion of cytokines. Although the entire intracellular signaling domain can usually be used, it should be understood that any truncated portion of the intracellular signaling domain sufficient to transduce the effector function signal can be used. In some embodiments, the CAR of the present invention comprises a signaling domain having an immunoreceptor tyrosine activation motif (ITAM), examples of which include signaling domains derived from primary signaling domains FcRγ, FcRβ, CD3γ, CD3δ, CD3ε, CD3ζ, CD22, CD79a, CD79b, and CD66d. In some embodiments, the CAR of the present invention comprises a CD3ζ intracellular signaling domain. In some embodiments, the CD32 intracellular signaling domain comprises the amino acid sequence shown in SEQ ID NO:159.

In some embodiments, the intracellular portion of the CAR of the present invention may further comprise a co-stimulatory domain. The term “co-stimulatory domain” refers to the intracellular domain of an immune co-stimulatory molecule, which is a cell surface molecule required for the effective response of lymphocytes to antigens. In some embodiments of the present invention, CAR may comprise a co-stimulatory domain derived from a protein selected from the group consisting of TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM and ZAP70. In some embodiments, the CAR of the present invention comprises a 4-1BB co-stimulatory domain. In some embodiments, the 4-1BB co-stimulatory domain comprises the amino acid sequence shown in SEQ ID NO:157.

The term “hinge region” refers to the connecting region between the binding domain and the transmembrane domain. In some embodiments of the present invention, CAR may comprise a hinge region or a combination thereof derived from a protein selected from the group consisting of CD8, CD28, IgG, 4-1BB, CD4, CD27, CD7, PD-1, and CH2CH3. In some embodiments, the CAR of the present invention comprises a CD8 hinge region. In some embodiments, the CD8 hinge region comprises an amino acid sequence as shown in SEQ ID NO:153.

In some embodiments, the CAR of the present invention may also comprise a signal peptide.

The term “signal peptide” refers to a leader sequence located at the amino terminus (N-terminus) of a polypeptide, which guides the polypeptide to the endoplasmic reticulum during or after translation. In some embodiments of the present invention, the signal peptide may comprise a signal peptide or a combination thereof derived from a protein selected from the group consisting of CD8, 4-1BB, GM-CSF, CD3γ, CD38, CD38, CD22, CD79a, CD79b, and CD66d. In some embodiments, the CAR of the present invention comprises a CD8 signal peptide. In some embodiments, the CD8 hinge region comprises an amino acid sequence as shown in SEQ ID NO:151.

In some embodiments, the CAR targeting CD7 of the present invention comprises amino acid sequences shown in SEQ ID NOs: 37, 42, 46, 50, 54, 58, 62, 66, 70, 74, 78, 82 or 86.

The term “specific binding” means that the binding domain of the chimeric antigen receptor of the present invention does not or substantially does not cross-react with any polypeptide other than the target antigen. The degree of specificity can be determined by immunological techniques, including but not limited to immunoblotting, immunoaffinity chromatography, flow cytometry, etc.

In some embodiments, the modified T cells of the present invention also express lymphocyte-antigen presenting cell co-stimulator (LACO-Stim). For LACO-Stim, see CN115052902A, which is incorporated herein by reference in its entirety. LACO-Stim is a fusion protein, the expression of which can not only promote the proliferation and activation of immune effector cells (such as T cells), but also stimulate the maturation and epitope spreading activity of antigen presenting cells.

The LACO-Stim comprises a first domain for activating antigen presenting cells (APC) and a second domain for activating immune effector cells, wherein

The first domain comprises: (i) a ligand that binds to an activating receptor of the APC, or a receptor binding fragment thereof, or (ii) an antibody that binds to an activating receptor of the APC, or an antigen-binding fragment thereof; and

The second domain comprises: (i) a co-stimulatory receptor of the immune effector cell, or a functional fragment thereof, (ii) a co-stimulatory ligand of the immune effector cell, or a receptor binding fragment thereof, or (iii) an antibody that binds to the co-stimulatory receptor of the immune effector cell, or an antigen-binding fragment thereof.

In some embodiments, the C-terminus of the first domain is connected to the N-terminus of the second domain. In some embodiments, the N-terminus of the first domain is connected to the C-terminus of the second domain.

In some embodiments, the first domain and the second domain are connected via a linker. The linker can be a flexible linker or a rigid linker. In some embodiments, the linker is a CD28 hinge region. In some embodiments, the CD28 hinge region comprises the amino acid sequence shown in SEQ ID NO:164.

APC refers to any cell that displays one or more antigens on its surface, for example, in combination with one or more major histocompatibility complex (MHC) proteins. The MHC/antigen complex can be recognized by T cells using their T cell receptors (TCRs) and elicit an immune response. APCs include, for example, dendritic cells (DCs), macrophages, monocytes, myeloid-derived suppressor cells, certain B cells, T cells, and Langerhans cells.

“Activating receptor” refers to a membrane protein expressed on APC, which, after binding to a ligand or antibody, can trigger a signal to promote the migration, differentiation, proliferation and/or activation of APC. APC activating receptors include, for example, CD40, CD80, CD86, CD91, DEC-205 and DC-SIGN. In some embodiments, the APC activating receptor is CD40.

A “ligand” of a receptor refers to a molecule that selectively binds to a receptor. In some embodiments, the ligand is a polypeptide. A “receptor binding fragment” of a ligand refers to a fragment of a ligand that retains its receptor binding ability.

In some embodiments, the first domain comprises an antibody that binds to an activating receptor of an APC, or an antigen-binding fragment thereof. In some embodiments, the first domain comprises an anti-CD40 antibody. In some embodiments, the antibody comprised by the first domain is an scFv, such as an anti-CD40 scFv. In some embodiments, the anti-CD40 scFv comprises the amino acid sequence shown in SEQ ID NO:163.

“Immune effector cell” refers to a cell with hematopoietic origin and plays a direct role in the immune response to a target (such as a pathogen, a cancer cell or a foreign substance). Immune effector cells include T cells, B cells, natural killer (NK) cells, NKT cells, macrophages, granulocytes, neutrophils, eosinophils, mast cells and basophils. In some embodiments, the second domain of the activation immune effector cell of the fusion protein provided by the present invention comprises a co-stimulatory receptor of the immune effector cell. In some embodiments, the immune effector cell is a T cell, a NK cell, a NKT cell, a macrophage, a neutrophil or a granulocyte. In some embodiments, the immune effector cell is a T cell.

“Stimulation” of immune effector cells refers to the primary response induced by the binding of stimulatory molecules to their cognate ligands, thereby mediating signal transduction events in immune effector cells, which can change the expression of certain genes and/or the reorganization of cytoskeletal structures, etc. “Stimulatory molecules” of immune effector cells refer to a molecule on immune effector cells that, after binding to cognate ligands usually present on APCs, can mediate signal transduction to promote the maturation, differentiation, proliferation and/or activation of immune effector cells.

As used in the present invention and as understood in the art, “co-stimulatory signal” refers to the signal from co-stimulatory receptors (such as CD28 or 4-1BB), which is combined with primary signals (such as TCR/CD3) to promote the best clonal expansion, differentiation and effector function of immune effector cells (such as T cells). As used in the present invention and as understood in the art, “co-stimulatory receptor” of immune effector cells refers to the molecule of the co-stimulatory response of immune effector cells specifically combined with “co-stimulatory ligand” on immune effector cells, and the co-stimulatory response is such as enhancing the activation or proliferation of immune effector cells. The co-stimulatory receptors of immune effector cells include but are not limited to CD28, 4-1BB, ICOS, CD27, OX40, DAP10, CD30, 2B4, CD2, LIGHT, GITR, TLR, DR3 and CD43. The “functional fragment” of co-stimulatory receptors is a fragment of co-stimulatory receptors, which retains the function of co-stimulatory receptors mediating co-stimulatory signals and stimulating immune effector cells.

In some embodiments, the co-stimulatory receptor is CD28. In some embodiments, the second domain comprises a CD28 polypeptide or a functional fragment thereof. In some embodiments, the second domain comprises the intracellular domain of CD28. In some embodiments, the intracellular domain of CD28 comprises the amino acid sequence as shown in SEQ ID NO:166. In some embodiments, the second domain further comprises a transmembrane domain of a co-stimulatory receptor, such as a CD28 transmembrane domain.

In some embodiments, the CD28 transmembrane domain comprises the amino acid sequence as shown in SEQ ID NO:165.

In some embodiments, the LACO-Stim of the present invention may further comprise a signal peptide. In some embodiments of the present invention, the signal peptide of LACO-Stim may comprise a signal peptide or a combination thereof derived from a protein selected from the group consisting of CD8, 4-1BB, GM-CSF, CD3γ, CD38, CD38, CD22, CD79a, CD79b and CD66d. In some embodiments, the LACO-Stim of the present invention comprises a CD8 signal peptide. In some embodiments, the CD8 hinge region comprises an amino acid sequence as shown in SEQ ID NO:151.

In some embodiments, the LACO-Stim comprises the amino acid sequence shown in SEQ ID NO:161.

Nucleic acids encoding chimeric antigen receptors targeting CD7 and/or nucleic acids encoding LACO-Stim can be introduced into T cells by methods known to those skilled in the art. For example, nucleic acids encoding chimeric antigen receptors can be introduced into T cells via vectors. Non-limiting examples of methods for introducing nucleic acids encoding chimeric antigen receptors into cells include: liposome transfection, calcium phosphate transfection, microinjection, electroporation, cell squeezing, sonication, protoplast fusion, puncture infection, gene gun, magnetic transfection, viral vector transduction, plasmid vector transfection, etc.

The nucleic acid encoding the chimeric antigen receptor targeting CD7 and the nucleic acid encoding LACO-Stim can be in the same expression cassette in T cells and driven by the same promoter, or the two can be in different expression cassettes and driven by their respective promoters. When they are in different expression cassettes, the promoters used by them can be the same or different. The chimeric antigen receptor targeting CD7 and LACO-Stim can be expressed as independent proteins in T cells, or can be expressed as a fusion protein of the two. The chimeric antigen receptor targeting CD7 and LACO-Stim can be connected via a linker. The linker can be a cleavable linker, such as a 2A linker (self-cleaving peptide), such as P2A, T2A or F2A. In some embodiments, F2A comprises the amino acid sequence shown in SEQ ID NO:179.

The nucleic acid encoding the chimeric antigen receptor targeting CD7 and the nucleic acid encoding the LACO-Stim can be introduced into T cells through the same vector or through different vectors. When the two are contained in the same vector, they can be in the same expression cassette and driven by the same promoter, or they can be in different expression cassettes and driven by their respective promoters. When they are in different expression cassettes, the promoters used by them can be the same or different.

The term “expression cassette” refers to a nucleic acid sequence that contains the complete elements required to express a gene, including the coding sequence of the gene and the coding sequence of regulatory sequences sufficient to direct the transcription of the gene. The expression cassette can be incorporated into a plasmid, chromosome, mitochondrial DNA, plastid DNA, virus or nucleic acid fragment.

In some embodiments, nucleic acid encoding a chimeric antigen receptor targeting CD7 and/or nucleic acid encoding LACO-Stim is introduced into T cells via a vector. The vector includes, but is not limited to, a plasmid vector, a viral vector, an autonomously replicating sequence, and a transposable element. In some embodiments, a viral vector, such as an adeno-associated virus (AAV) vector, an adenovirus vector, a retrovirus (including a lentivirus) vector, a herpes virus (e.g., herpes simplex virus) vector, a poxvirus vector, a baculovirus vector, or a papillomavirus vector, is used to introduce nucleic acid encoding a chimeric antigen receptor into T cells. In some embodiments, an AAV vector is used to introduce a chimeric antigen receptor targeting CD7 into T cells.

AAV vectors are replication-deficient vectors that can infect dividing cells and non-dividing cells and can integrate their genomes into the genomes of host cells. Recombinant AAV (rAAV) includes at least transgenes and regulatory sequences, as well as 5′ and 3′ AAV inverted terminal repeats (ITRs). AAV has a variety of serotypes, and in some embodiments, rAAV can comprise ITRs and capsid sequences separated from AAV1, AAV2, AAV3, AAV4, AAV5, AAV6, AAV7, AAV8, AAV9, AAV10, AAV11, AAV 12, AAV13, AAV 14, AAV15 or AAV16. In some embodiments, chimeric AAV vectors can be used, i.e., ITR sequences and capsid sequences are respectively derived from different AAV serotypes.

The vector may generally contain regulatory elements, such as replication origin, promoter, enhancer, translation initiation signal, post-transcriptional regulatory elements, polyadenylation sequence and/or 5′ and 3′ untranslated regions, etc. These regulatory elements interact with host cell proteins to perform transcription and translation. The selection and use of different regulatory elements are well known to those skilled in the art.

The term “promoter” refers to a recognition site of a polynucleotide (DNA or RNA) to which RNA polymerase binds. A promoter is usually operably linked to a gene to be expressed to drive the expression of the gene. In the present invention, the expression of a chimeric antigen receptor targeting CD7 and/or LACO-Stim in T cells can be driven by an endogenous promoter of T cells, or by a promoter exogenous to T cells.

In some embodiments, nucleic acid encoding a chimeric antigen receptor targeting CD7 and/or nucleic acid encoding LACO-Stim can be inserted into a position operably linked to an endogenous promoter of a T cell, thereby driving its expression through the endogenous promoter. A polynucleotide comprising a nucleic acid encoding a chimeric antigen receptor targeting CD7 and/or a nucleic acid encoding LACO-Stim can be designed to insert the nucleic acid encoding the chimeric antigen receptor into a position operably linked to an endogenous promoter of a T cell. For example, nucleic acid encoding a chimeric antigen receptor targeting CD7 and/or nucleic acid encoding LACO-Stim can have homology arms homologous to endogenous gene sequences of T cells on both sides, thereby being integrated into a position operably linked to an endogenous promoter of a T cell through homology directed recombination (HDR). In some embodiments, the endogenous promoter can be a TRAC promoter, a B2M promoter, or a CD7 promoter endogenous to a T cell.

In some embodiments, nucleic acid encoding a chimeric antigen receptor targeting CD7 and/or nucleic acid encoding LACO-Stim can be operably linked to a promoter exogenous to T cells, wherein the exogenous promoter drives expression thereof. For example, nucleic acid encoding a chimeric antigen receptor targeting CD7 and/or nucleic acid encoding LACO-Stim can be introduced into T cells as part of a vector, wherein the nucleic acid(s) is/are operably linked to an exogenous promoter. The promoter that is exogenous to T cells includes, but is not limited to, cytomegalovirus (CMV) immediate early promoter, simian virus 40 (SV40) promoter, Moloney murine leukemia virus (MoMLV) LTR promoter, herpes simplex virus (HSV) (thymidine kinase) promoter, H5, P7.5 and P11 promoters derived from vaccinia virus, short elongation factor 1-α(EF1a-short) promoter, long elongation factor 1-α(EF1a-long) promoter, ubiquitin C(UBC) promoter, phosphoglycerate kinase-1 (PGK) promoter, cytomegalovirus enhancer/chicken β-actin (CAG) promoter, β-actin promoter, etc. In some embodiments, the promoter that is exogenous to T cells is phosphoglycerate kinase-1 (PGK) promoter. In some embodiments, the PGK promoter comprises a sequence as shown in SEQ ID NO:176.

The term “operably linked” means that a regulatory sequence (e.g., a promoter) is positioned relative to a coding sequence in a manner enabling the regulatory sequence (e.g., a promoter) to direct the production of the polypeptide encoded by the coding sequence.

The term “endogenous” refers to a nucleic acid sequence or amino acid sequence that occurs naturally within a T cell prior to the introduction of a modification described herein.

The term “exogenous” refers to a nucleic acid sequence or amino acid sequence that is not naturally present or expressed in T cells.

A polynucleotide comprising a nucleic acid encoding a chimeric antigen receptor targeting CD7 and/or a nucleic acid encoding LACO-Stim can be engineered to insert a nucleic acid encoding a chimeric antigen receptor targeting CD7 and/or a nucleic acid encoding LACO-Stim into a target gene locus in a T cell, such as a safe harbor locus. In the present invention, a nucleic acid encoding a chimeric antigen receptor targeting CD7 and/or a nucleic acid encoding LACO-Stim can be inserted into a TRAC, B2M or CD7 locus in a T cell to disrupt the TRAC, B2M or CD7 gene and express the chimeric antigen receptor. Such site-directed insertion can be achieved, for example, by homologous directed recombination.

In some embodiments, nucleic acid encoding a chimeric antigen receptor targeting CD7 and/or nucleic acid encoding LACO-Stim can be comprised in the donor repair template of the CRISPR/Cas system, nucleic acid encoding a chimeric antigen receptor targeting CD7 and/or nucleic acid encoding LACO-Stim is located between two homology arms of the donor repair template, and inserted near the Cas protein cleaving site by homology-directed recombination to disrupt the TRAC, B2M or CD7 genes in T cells and express chimeric antigen receptor and/or LACO-Stim. In some embodiments, nucleic acid encoding a chimeric antigen receptor targeting CD7 and/or nucleic acid encoding LACO-Stim is included in the donor repair template of the CRISPR/Cas system targeting the TRAC gene and inserted into the endogenous TRAC gene of the T cell.

In some embodiments, a donor repair template comprising a nucleic acid encoding a chimeric antigen receptor targeting CD7 and/or a nucleic acid encoding a LACO-Stim is contained in a vector, such as an AAV vector, and introduced into T cells as part of the vector.

In some embodiments, the modified T cells of the present invention can be used as drugs for treating diseases associated with the expression of CD7, which can be formulated as pharmaceutical compositions.

Diseases associated with CD7 expression include non-solid tumors (e.g., hematological tumors, such as leukemias and lymphomas) and solid tumors. Hematological tumors are cancers of the blood or bone marrow and include, but are not limited to, acute leukemias (such as acute lymphocytic leukemia (ALL), acute myeloid leukemia (AML), acute myeloid leukemia and myeloblastic, promyelocytic, myelo-monocytic, monocytic and erythroleukemias), chronic leukemias (such as chronic myeloid (granulocytic) leukemia, chronic myeloid leukemia and chronic lymphocytic leukemia), polycythemia vera, lymphoma, Hodgkin lymphoma, non-Hodgkin lymphoma (indolent and high-grade forms), multiple myeloma, Waldenstrom's macroglobulinemia, myelodysplastic syndrome, hairy cell leukemia, Burkitt lymphoma, diffuse large cell lymphoma, mantle cell lymphoma, T-lymphoblastic lymphoma (T-LBL), early pre-T lymphoblastic leukemia (ETP-ALL), extranodal NK/T-cell lymphoma, small lymphocytic lymphoma (SLL), and myelodysplastic syndromes. A solid tumor is an abnormal mass of tissue that typically does not contain cysts or liquid areas, which can be benign or malignant. Different types of solid tumors are named according to the cell types from which they form (such as sarcoma, carcinoma, and lymphoma). Examples of solid tumors include, but are not limited to: fibrosarcoma, myxosarcoma, liposarcoma mesothelioma, pancreatic cancer, ovarian cancer, peritoneum, omentum and mesenteric cancer, pharyngeal cancer, prostate cancer, rectal cancer, renal cancer, skin cancer, small intestine cancer, melanoma, renal cancer, laryngeal cancer, soft tissue cancer, gastric cancer, testicular cancer, colon cancer, esophageal cancer, cervical cancer, alveolar rhabdomyosarcoma, bladder cancer, bone cancer, brain cancer, breast cancer, anal cancer, eye cancer, intrahepatic bile duct cancer, joint cancer, cervical cancer, gallbladder cancer, pleural cancer, nasal cancer, middle ear cancer, oral cancer, vulvar cancer, thyroid cancer and ureteral cancer. In some embodiments, the disease associated with CD7 expression is T-cell acute lymphoblastic leukemia (T-ALL) or T-lymphoblastic lymphoma.

The terms “subject” and “patient” are used interchangeably herein. The term “subject” as used herein refers to any organism to which the antibodies or antigen-binding fragments thereof of the present invention can be administered, e.g., for experimental, diagnostic, preventive and/or therapeutic purposes. Typical subjects include animals (e.g., mammals, such as mice, rats, rabbits, non-human primates, such as chimpanzees and other apes, and humans). Subjects can be mammals, particularly humans, including females (women) or males (men), and include newborns, infants, teenagers, youths, adults or the elderly, and further include various races and ethnicities. In some embodiments, a subject refers to an individual in need of diagnosis, treatment or prevention of a disease or condition, and the subject may suffer from the disease or condition, or have the risk of suffering from the disease or condition.

The term “treatment” as used herein refers to providing a beneficial or desired clinical outcome to a disease, such as eliminating the disease, alleviating symptoms, reducing the extent of the disease, stabilizing, improving or alleviating the state of the disease, or slowing down the progression of the disease. Measurement of treatment outcomes can be based on, for example, the results of physical examinations, pathological tests, and/or diagnostic tests known in the art. Treatment may also refer to extending the survival period compared to the expected survival period when the subject does not receive treatment. Treatment may also refer to reducing the incidence or morbidity of the disease, or its recurrence, compared to the disease that would occur if the measure was not taken. Clinically, this treatment may also be referred to as prevention.

The terms “pharmaceutical composition” and “pharmaceutical preparation” of the present invention can be used interchangeably. The pharmaceutical composition may contain a pharmaceutically acceptable carrier. The term “pharmaceutically acceptable carrier” used herein refers to any carrier contained in a pharmaceutical composition as an inactive ingredient, which gives the pharmaceutical composition an appearance and properties suitable for administration. Pharmaceutically acceptable carriers have substantially no long-term or permanent adverse effects when administered to a subject, such as stabilizers, diluents, additives, adjuvants, excipients, etc. “Pharmaceutically acceptable carriers” should be a pharmaceutically inert material, substantially without biological activity, and constitute the main part of the preparation.

The pharmaceutical composition of the present invention can be formulated into various modes of administration according to techniques known in the art. For example, see Remington, The Science and Practice of Pharmacy (9th Ed. 1995). When manufacturing the pharmaceutical composition, the active agent is usually mixed with a pharmaceutically acceptable carrier, etc. Of course, the pharmaceutically acceptable carrier must be acceptable, i.e. compatible with any other ingredients in the formulation, and must be harmless to the subject. Pharmaceutically acceptable carriers may include, but are not limited to, buffers, excipients, stabilizers, preservatives, wetting agents, surfactants, emulsifiers, or combinations thereof. Examples of buffers include, but are not limited to, acetic acid, citric acid, histidine, boric acid, formic acid, succinic acid, phosphoric acid, carbonic acid, malic acid, aspartic acid, Tris buffer, HEPPSO, HEPES, neutral buffered saline, phosphate buffered saline, etc.

The pharmaceutical composition of the present invention can be administered in any manner suitable for the disease to be treated (or prevented) and the subject. In certain embodiments, the mode of administration may include, but is not limited to, parenteral or non-parenteral routes, including oral, sublingual, buccal, transdermal, rectal, vaginal, intradermal, intranasal or parenteral routes, such as intravenous injection (i.v.), intraperitoneal, intradermal, subcutaneous, intramuscular, intracranial, intrathecal, intratumoral, transdermal, transmucosal, intraarticular, intrathecal, intrathecal, intrahepatic, intraneural or intracranial injection or infusion. The pharmaceutical composition can be directly injected into a tumor, lymph node, tissue, organ or infection site.

Suitable oral dosage forms include, but are not limited to, tablets, capsules, powders, pills, granules, suspensions, solutions or preconcentrates of solutions, emulsions or preconcentrates of emulsions. Pharmaceutically acceptable carriers that can be used for oral dosage forms include water, ethylene glycol, oil, alcohol, flavoring agents, preservatives, colorants, etc. Carriers such as starch, sugar, microcrystalline cellulose, diluents, fillers, glidants, granulating agents, lubricants, binders, stabilizers, disintegrants, etc. can be used to prepare oral solid preparations, such as powders, capsules or tablets.

Dosage forms suitable for parenteral administration include, but are not limited to, sterile liquid preparations, such as isotonic aqueous solutions, emulsions, suspensions, dispersions, or viscous compositions, which can be buffered to a desired pH. Parenteral dosage forms can be ready-to-use or can be dry products that are ready to be dissolved or suspended in a pharmaceutically acceptable carrier. Parenteral dosage forms can be sterile formulations or can be capable of being sterilized prior to administration to a subject. Pharmaceutically acceptable carriers that can be used to provide parenteral dosage forms include, but are not limited to, water for injection; aqueous carriers, such as, but not limited to, sodium chloride injection, Ringer's injection, dextrose injection. Water-soluble carriers, such as, but not limited to, ethanol, polyethylene glycol, and polypropylene glycol; non-aqueous carriers, such as, but not limited to, corn oil, cottonseed oil, peanut oil, sesame oil, ethyl oleate, isopropyl myristate, and benzyl benzoate; and solubilizers, such as cyclodextrins.

The modified T cells or pharmaceutical compositions of the present invention are administered to a subject in a therapeutically effective amount. The terms “therapeutically effective amount” and “effective amount” used herein are used interchangeably and refer to an amount that is effective at the necessary dosage and time period to achieve the desired therapeutic effect. The therapeutically effective amount may vary according to different factors, such as disease state, age, sex, and individual weight, and the ability of a treatment method or combination of treatment methods to induce a desired response in an individual.

An effective amount may refer to an amount that causes a detectable change in biological or chemical activity. Detectable changes can be detected and/or further quantified by a person skilled in relevant art. In addition, an “effective amount” may specify an amount that maintains a desired physiological state, i.e., reduces or prevents a significant decline and/or promotes improvement in a condition.

The amount and frequency of dosing will be determined by factors such as the subject's condition (such as age, weight, sex, and the subject's response to the drug) and the type and severity of the subject's disease, although appropriate doses can be determined by clinical trials.

In the present invention, the effective amount of modified T cells can be, for example, 5×10⁶, 1×10⁷, 2×10⁷, 5×10⁷, 1×10⁸, 2×10⁸, 5×10⁸, 1×10⁹, 2×10⁹ or 5×10⁹ cells administered in a single administration.

The modified T cells or pharmaceutical compositions of the present invention can be administered once or twice a day; or once every 2, 3, 4, 5, 6, 7, 8, 9 or 10 days, once every 1, 2, 3, 4, 5 or 6 weeks, or once every 1, 2, 3, 4, 5 or 6 months or longer. The pharmaceutical composition may also be administered several times a week (such as 1, 2, 3, 4 or 5 times) or several times a month (such as 1, 2, 3, 4, 5, 6, 7, 8, 9 or 10 times). For example, in a 5-times-a-week regimen, the pharmaceutical composition can be administered once a day for 5 consecutive days, followed by two consecutive days of rest.

The modified T cells or pharmaceutical compositions of the present invention can be used in combination with other drugs and treatment methods, such as other anti-tumor drugs, chemotherapy or radiotherapy. “Combined use” as mentioned here refers to the administration of two (or more) different drugs and/or therapies to a subject during the treatment process.

The two or more drugs and/or therapies in the combination can be administered by different routes and regimens. Two or more drugs and/or treatment methods can be administered to the subject simultaneously or sequentially. In some embodiments, when the administration of the second drug or therapy begins, the administration of one drug or therapy is still in progress, so there is overlap in the administration. Such a regimen can be referred to as “simultaneous” herein. When administered simultaneously, two or more drugs and/or therapies can be formulated together into a single dosage form, or formulated into two or more independent dosage forms.

### EXAMPLE

Unless otherwise specified, the methods and materials of the examples described below are conventional products that can be purchased from the market.

### Example 1: In Vitro Transcription (IVT) Preparation and Purification of gRNA

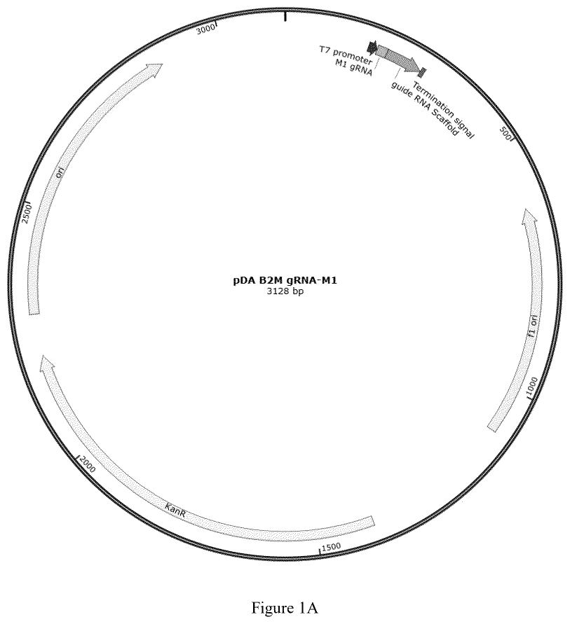

1. Design 22 B2M-targeting gRNAs, each containing a guide sequence as shown in SEQ ID NOs: 1-22. Construct plasmids pDA-T7-B2M gRNA for in vitro transcription of these B2M gRNAs, respectively. Using the plasmid carrying B2M gRNA-1 (designated as pDA-B2M-gRNA-M1; see FIG. 1 for plasmid map) as an example. Linearize the 22 pDA-T7-B2M gRNA plasmids by Hind III restriction endonuclease.
2. Purify the linearized vector using a PCR cleanup kit (Qiagen) and elute with RNase-free water.
3. DNA concentration was measured by Nanodrop and checked by running an agarose DNA gel.
4. Perform in vitro transcription (IVT) according to the manufacturer's standard operating procedure (Thermofisher, Cat. No. AMB13345). Briefly, 1 μg of template DNA, ATP buffer, UTP buffer, CTP buffer, GTP buffer, 10×reaction buffer, T7 enzyme, and RNase-free H₂O were added to a 0.2 mL PCR tube in a volume of 20 ul and incubated at 37° C. for 4 hours.
5. After 4 hours, add 2 μl of DNase I to each reaction and react at 37° C. for 15 minutes.
6. Purify IVT gRNA using the RNasy kit (Qiagen).
7. The concentration of purified gRNA was measured by Nanodrop and checked by PAGE gel.

### Example 2: Culture of CCRF-CEM-GFP Tumor Cells and T Cells

CCRF-CEM-GFP tumor cell line was cultured in RPMI-1640 medium supplemented with 10% fetal bovine serum and 1% penicillin-streptomycin (P/S), with subculturing performed every 2-3 days. Primary CD3+ T cells isolated and purified from PBMC were activated with anti-CD3/CD28 Dynabeads (Thermofisher, catalog number: 402031) and cultured in R10 medium (RPMI-1640 basal medium supplemented with 10% fetal bovine serum, 1% penicillin-streptomycin (P/S), 1% HEPES, 1% sodium pyruvate, 1% Glutamax and 1% non-essential amino acids (NEAA). On day 4, the magnetic beads were removed from the T cells and the cells were cultured for subsequent electroporation of Cas9 protein/gRNA complexes and AAV virus infection.

### Example 3: Assessment of B2M Gene Knockout Efficiency by gRNA

1. Harvest T cells activated by anti-CD3/CD28 Dynabeads on day 4, remove beads, and wash three times with Opti-MEM medium.
2. Resuspend the cell pellet in Opti-MEM medium and adjust the cell density to 6×10⁷/mL.
3. 7.5 μg of each B2M gRNA obtained in Example 1 was mixed with 15 μg TrueCut™ Cas9 Protein v2 (Thermofisher, catalog number: A36499) evenly in a 1.5 mL EP tube and incubated at room temperature for 10 minutes. After 10 minutes, 100 μL of T cells were added to the Cas9 protein/gRNA mixture, gently mixed, and immediately electroporated.
4. Set parameters on BTX ECM 830 machine: 360 voltage, 1 millisecond.
5. Add 100 μL of cells mixed with Cas9 protein/gRNA into the BTX electroporation cuvette and tap gently to avoid bubbles.
6. Perform electroporation, then transfer the electroporated T cells into 1 mL of pre-warmed culture medium, mix well, and then place in a 37° C. incubator for further culture.
7. On the 4th day after electroporation, harvest approximately 0.5×10⁶ cells and analyze them by flow cytometry staining using anti-B2M antibody to detect the gene knockout efficiency of B2M.

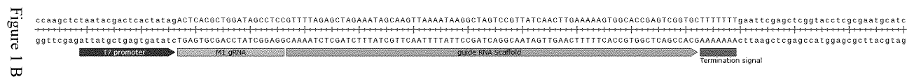

The results are shown in FIG. 2. B2M gRNA-15 and gRNA-22 have the highest gene knockout efficiency, reaching 79.1% and 80.2%, respectively, and were selected for subsequent experiments.

### Example 4: Testing of TriKO T Cell Preparation Conditions

1. Harvest T cells activated with anti-CD3/CD28 Dynabeads on day 4, remove beads, and wash them three times with Opti-MEM medium.
2. Resuspend the cell pellet in Opti-MEM medium and adjust the cell density to 6×10⁷/mL.
3. 6 μg of different combinations of TRAC gRNA/B2M gRNA/CD7 gRNA (all obtained by in vitro transcription according to the method described in Example 1) was mixed with 24 μg of TrueCut™ Cas9 Protein v2 (Thermofisher, catalog number: A36499) evenly in a 1.5 mL EP tube and incubated at room temperature for 10 minutes. After 10 minutes, 100 μL of T cells were added to the Cas9 protein/gRNA mixture, gently mixed, and immediately electroporated.
4. Set parameters on the BTX ECM 830 machine: 360 voltage, 1 millisecond.
5. Add 100 μL of cells mixed with Cas9 protein/gRNA into the BTX electroporation cuvette and tap gently to avoid bubbles.
6. Perform electroporation, then transfer the electroporated T cells into 1 mL of pre-warmed culture medium, mix well, and then place in a 37° C. incubator for further culture.
7. On the 4th day after electroporation, detect the gene knockout efficiency of TCR/CD7/B2M.

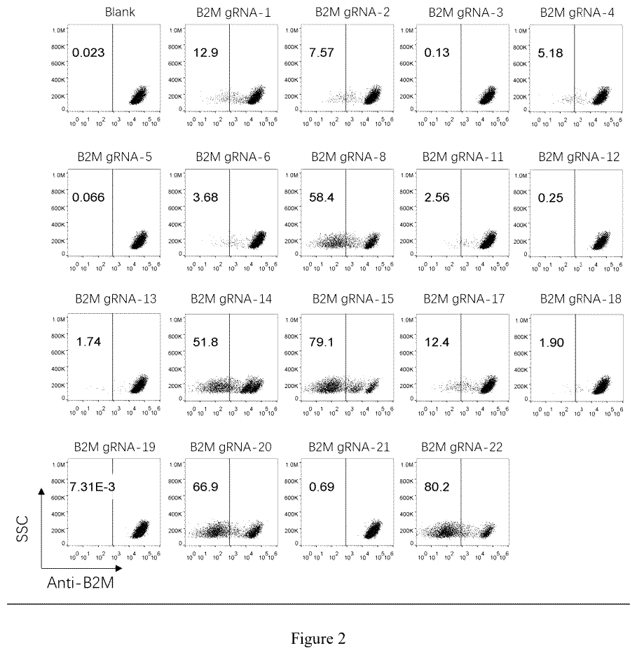

The results are shown in FIG. 3. For TCR knockout, the knockout efficiency of TRAC gRNA 3.4 is higher than that of TRAC gRNA 19.6, and for B2M knockout, the knockout efficiency of B2M gRNA-15 is higher than that of B2M gRNA-22. For TRAC/B2M/CD7, the best gRNA combination for simultaneous knockout of the three genes is TRAC gRNA3.4/B2M gRNA-15/CD7 gRNA-11.

### Example 5: Anti-CD7 Antibodies and Chimeric Antigen Receptors Targeting CD7

1. Infect the logarithmic phase TG1 library culture with freshly thawed M13K07 helper phage at a multiplicity of infection of 20:1 (phage-to-cell ratio) and induce the expression of the fully human antibody phage display library with IPTG overnight.
2. Purify the phage library by PEG/NaCl precipitation and determine the phage titer. Store the phage at 4° C. and proceed to scFv selection as soon as possible.
3. Selection of CD7-specific scFv-phage. For the first round of selection, 20 μg/mL CD7-6His protein dissolved in 1×PBS was coated on Maxisorp plates and incubated overnight at 4° C. (For subsequent rounds, the protein concentration was reduced for more stringent selection, 2 μg/mL in the second round of bio-panning and 0.5 μg/mL in the third round of bio-panning).
4. Wash three times with PBS, add blocking buffer (5% milk+1% BSA in 1×PBS) to each well and incubate at RT for 2 h.
5. Discard the blocking buffer, add the phage solution, seal with sealing film, and incubate with gentle shaking for 2 h.
6. In the first round of screening, wash 10 times with PBST. In the following rounds, increase the stringency of washing by increasing the number of washing cycles (20 times in the second round and 30 times in the third round).
7. Elute the antigen-bound scFv phages. Add 1 mL of acidic elution buffer (pH2.2), incubate for about 8 minutes, then use a pipette to pipette the eluted phages into about 100 μL of neutralization buffer and place in a new PP tube.
8. Inoculate 15 mL of the logarithmic phase TG1 culture (OD600=0.5) with the eluted phage solution in a new 50 mL tube. Incubate at 37° C. statically for 30 minutes, followed by shaking incubation for 30 minutes. Then culture on a 15 cm plates containing 2×YT-GA agar.
9. Incubate the plates at 30° C. overnight and harvest the bacteria for subsequent screening.
10. After three rounds of selection, select hundreds of positive colonies for monoclonal phage ELISA (mpELISA) screening.
11. mpELISA screening. Phage supernatants produced by individual bacteria were incubated with pre-blocked Maxisorp plates coated with 2 μg/mL CD7-6His protein. After washing three times, 100 μL/well of HRP-conjugated anti-M13 antibody diluted 1:5000 in blocking buffer (5% milk+1% BSA in 1×PBS) was added and incubated for 60 minutes at RT. After washing the plate five times with PBST, 100 μL/well of TMB substrate solution was added and incubated for 10-30 minutes until blue color appeared. The reaction was terminated by adding 50 μL/well of stop solution (2N H2SO4). The absorbance at 450 nm was read on a microplate reader.
12. Positive clones were selected based on the ELISA results and used as templates for PCR cloning of scFv sequences (forward primer sequence: tgcagctggcacgacaggtttc (SEQ ID NO:30), reverse primer sequence: cgtcagactgtagcacgtt (SEQ ID NO:31)). The PCR products were then sequenced using the Sanger sequencing method (forward primer sequence: aacaattgaattcaggagga (SEQ ID NO:32), reverse primer sequence: cctcctaagaagcgtagtc (SEQ ID NO:33)).
13. The CDR regions of scFv were analyzed using the abysis website (http://abysis.org/) using the Chothia numbering system.

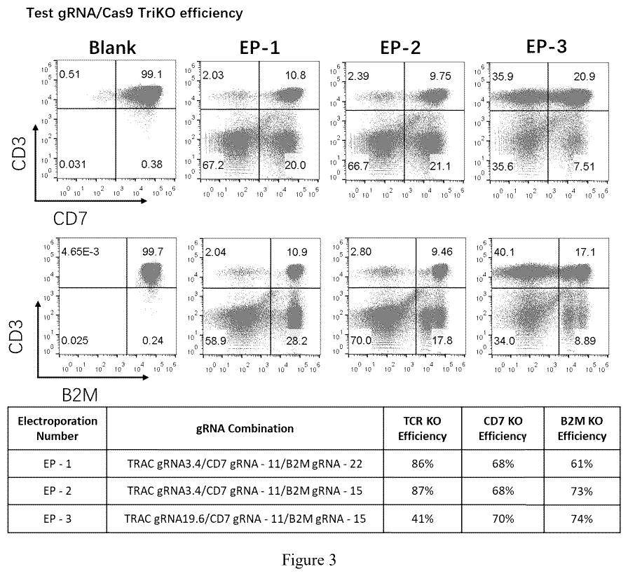

The results are shown in FIG. 4. After three rounds of bio-panning, 288 colonies were selected for culture and phage supernatant was produced to test its binding to CD7-Fc protein. The positive clones are highlighted in gray. 13 anti-CD7 scFv antibodies were screened, namely H1, H4, H6, H8, H9, H10, H12, H13, H17, H18, H5, H7, and H15.

| Number | Clone number of |
|---|---|
| of scFv | ELISA (FIG. 4) |
| H1 | 1-B8 |
| H4 | 1-H4 |
| H5 | 1-H10 |
| H6 | 2-B5 |
| H7 | 2-C11 |
| H8 | 2-D1 |
| H9 | 2-F10 |
| H10 | 2-H8 |
| H12 | 3-B6 |
| H13 | 3-C9 |
| H15 | 3-E1 |
| H17 | 3-H3 |
| H18 | 3-H10 |

These scFvs were used to construct CD7-targeting chimeric antigen receptor, which comprised, from N-terminus to C-terminus, CD8 signal peptide, anti-CD7 scFv, CD8 hinge region, CD8 transmembrane domain, 4-1BB co-stimulatory domain, and CD3ζ intracellular signaling domain. The corresponding chimeric antigen receptors are designated as CD7H1.BBZ, CD7H4.BBZ, CD7H6.BBZ, CD7H8.BBZ, CD7H9.BBZ, CD7H10.BBZ, CD7H12.BBZ, CD7H13.BBZ, CD7H17.BBZ, CD7H18.BBZ, CD7H5.BBZ, CD7H7.BBZ, and CD7H15.BBZ.

### Example 6: Construction of LACO-Stim Fusion Protein A40C2828

A40C2828 is a lymphocyte-antigen presenting cell co-stimulator (LACO-Stim) that co-expresses with a chimeric antigen receptor targeting CD7. A40C2828 is a fusion protein that comprises, from the N-terminus to the C-terminus, scFv A40C, CD28 hinge region, CD28 transmembrane domain and CD28 intracellular signaling domain.

### Example 7: Construction of AAV Plasmid

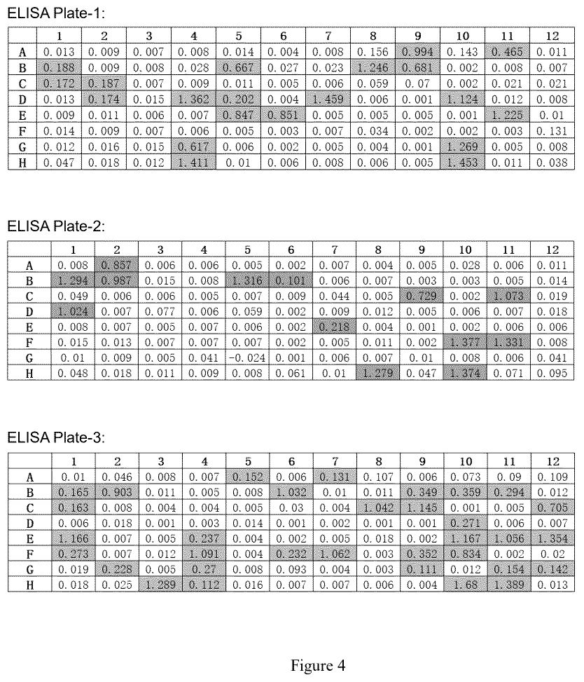

As shown in FIG. 5, the R196H1A plasmid (pAAV-HA gRNA19.6-CD7H1.BBZ-F2A-A40C2828) was designed for the TRAC gRNA19.6 gene knock-in site, which comprises the AAV ITR element, the homology arm sequences on both sides of the TRAC gRNA19.6 site, the P2A element, the CD7H1.BBZ-F2A-A40C2828 gene expression cassette, the BGH poly A signal, etc. Targeting the TRAC gRNA3.4 gene knock-in site, the R3H1A plasmid (pAAV-HA gRNA3.4-CD7H1.BBZ-F2A-A40C2828) was designed, which comprises the AAV ITR element, the homology arm sequences on both sides of the TRAC gRNA3.4 site, the P2A element, the CD7H1.BBZ-F2A-A40C2828 gene expression cassette, the BGH poly A signal, etc. For the knock-in site of TRAC gRNA3.4 gene, R3PGK plasmid (pAAV-HA gRNA3.4-PGK-CD7H1.BBZ-F2A-A40C2828) was further designed, which comprises AAV ITR elements, homology arm sequences on both sides of the TRAC gRNA3.4 site, PGK promoter elements, CD7H1.BBZ-F2A-A40C2828 gene expression cassette, the BGH poly A signal, etc. R196H1A and R3H1A use the endogenous promoter sequence of the TRAC gene, while R3PGK uses the exogenous promoter sequence of PGK.

### Example 8: Preparation of TriKO CD7 CAR-T Cells

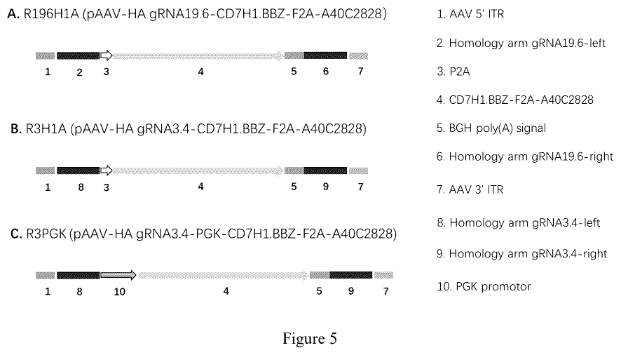

1. Harvest T cells activated with anti-CD3/CD28 Dynabeads on day 4, remove the beads, and wash them three times with Opti-MEM medium.
2. Resuspend the cell pellet in Opti-MEM medium and adjust the cell density to 6×10⁷/mL.
3. 6 μg of different combinations of TRAC gRNA/B2M gRNA/CD7 gRNA were mixed with 24 μg of TrueCut™ Cas9 Protein v2 (Thermofisher, catalog number: A36499) in a 1.5 mL EP tube and incubate at room temperature for 10 minutes. After 10 minutes, 100 μL of T cells were added to the Cas9 protein/gRNA mixture, mixed gently, and performed electroporation immediately.
4. The parameters were set on the BTX ECM 830 machine: 360 voltage, 1 millisecond.
5. 100 μL of cells mixed with Cas9 protein/gRNA was added into the BTX electroporation cuvette and tapped gently to avoid bubbles.
6. Electroporation was performed, then transferred the electroporated T cells to 450 μL of pre-warmed culture medium, mixede well, and divided into 5 wells of a 48-well plate at a volume of 100 μL per well. Then added the corresponding volume of AAV virus (Guangzhou Paizhen Biotechnology Co., Ltd.) to the electroporated T cells according to different MOIs (FIG. 6), mixed gently, and then placed in a 37° C. incubator for further culture.
7. After culturing at 37° C. for 2 h, 1 mL of R10 medium was added to each well and the culture was continued culturing in a 37° C. incubator.
8. The gene knockout efficiency of TCR/CD7/B2M and the expression efficiency of CAR were detected on the 4th and 8th days after electroporation, respectively.

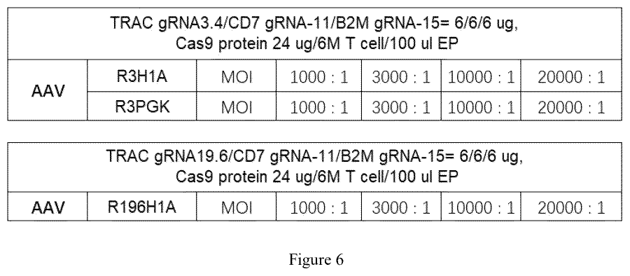

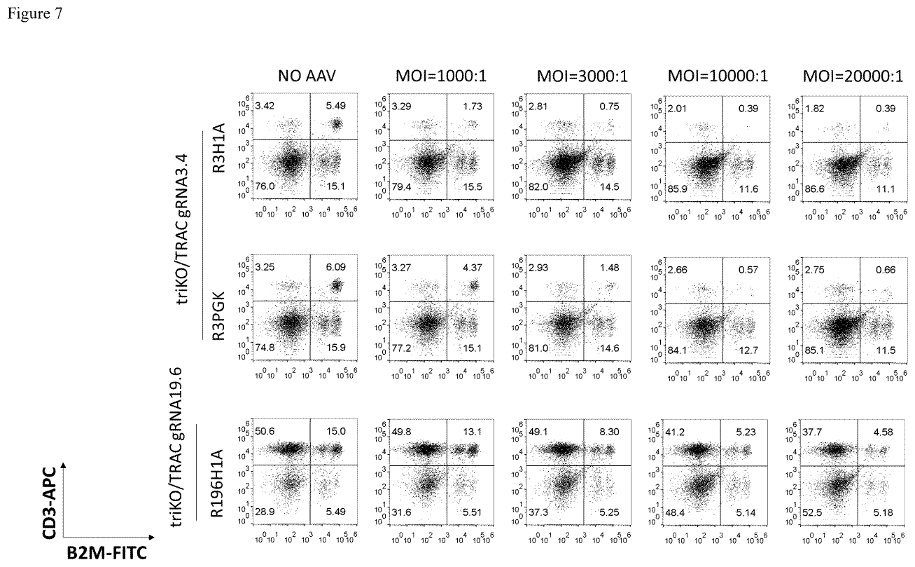

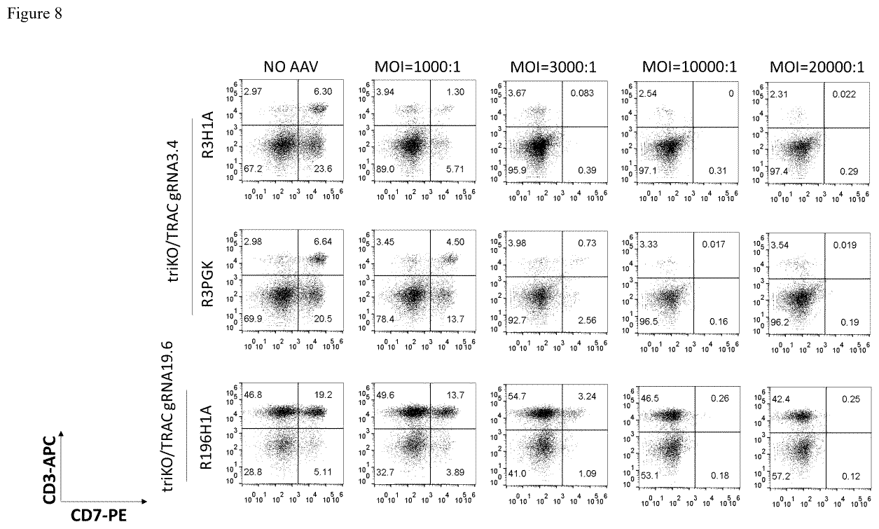

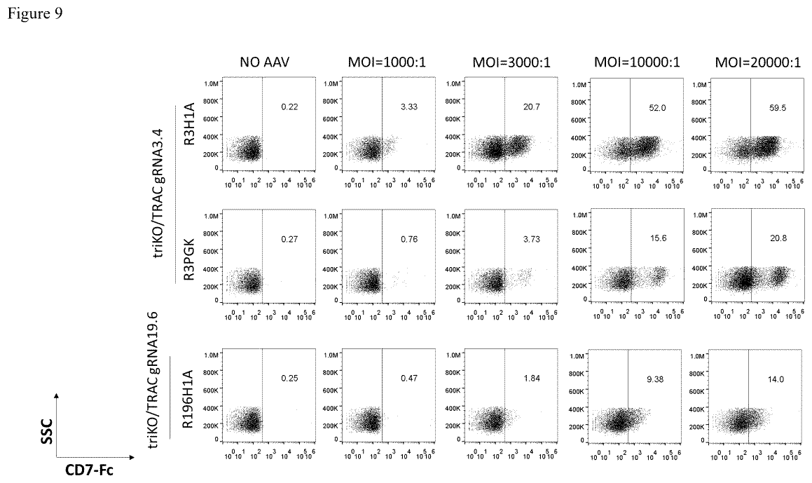

The results showed that on the 4th day after electroporation, the three genes (TCR/B2M/CD7) were all efficiently knocked out (FIGS. 7 and 8). At the same time, the expression of CD7 CAR and A40C2828 (LACO-stim) was also detected (FIGS. 9 and 10). In addition, the efficiency of gene knockout, the expression of CD7 CAR and the expression of A40C2828 were positively correlated with the MOI value added by AAV.

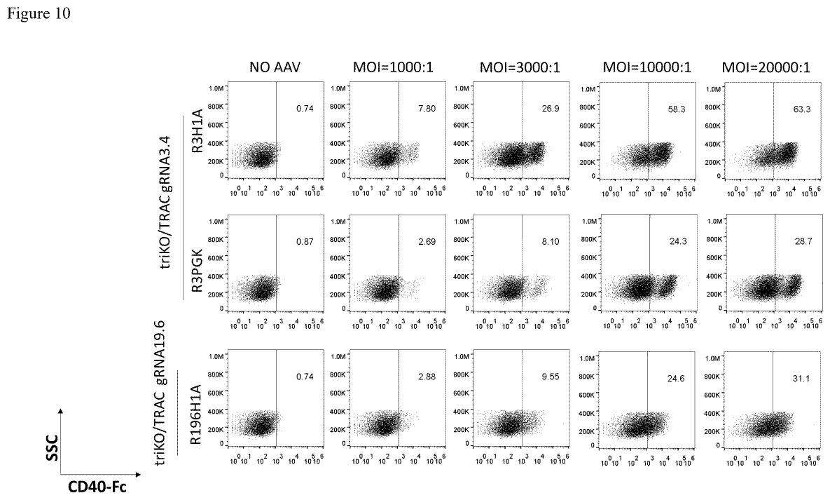

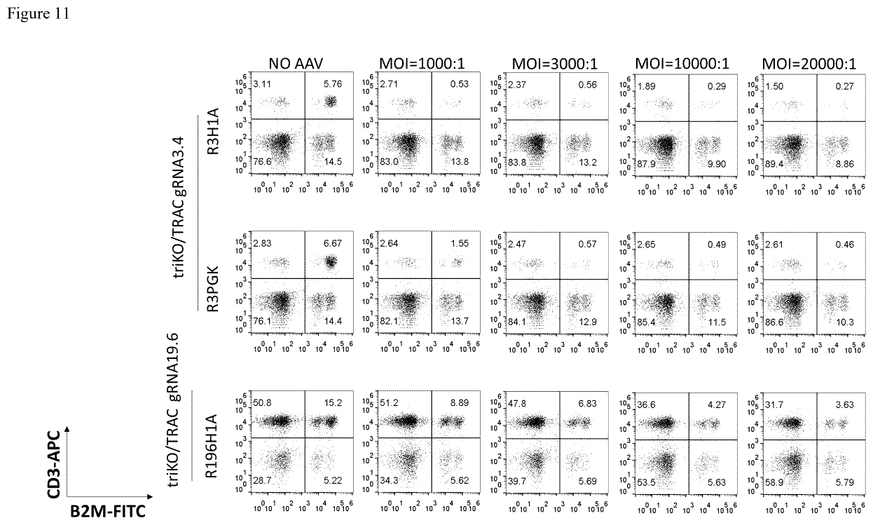

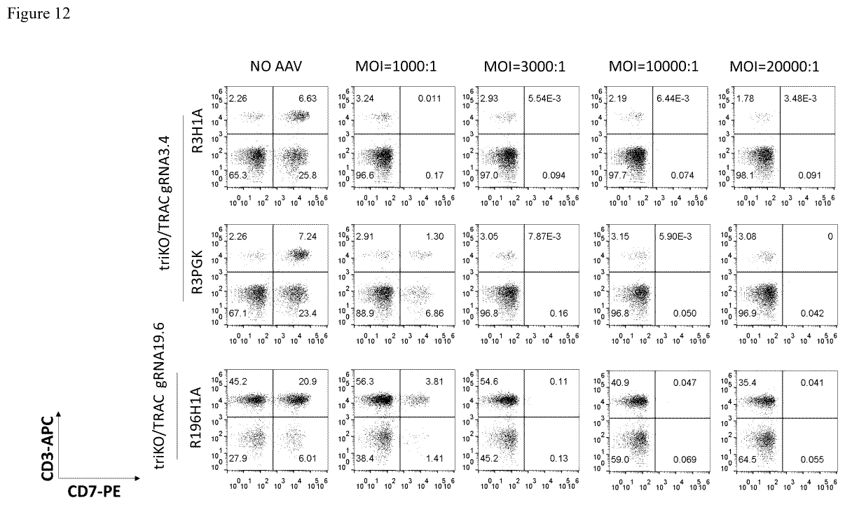

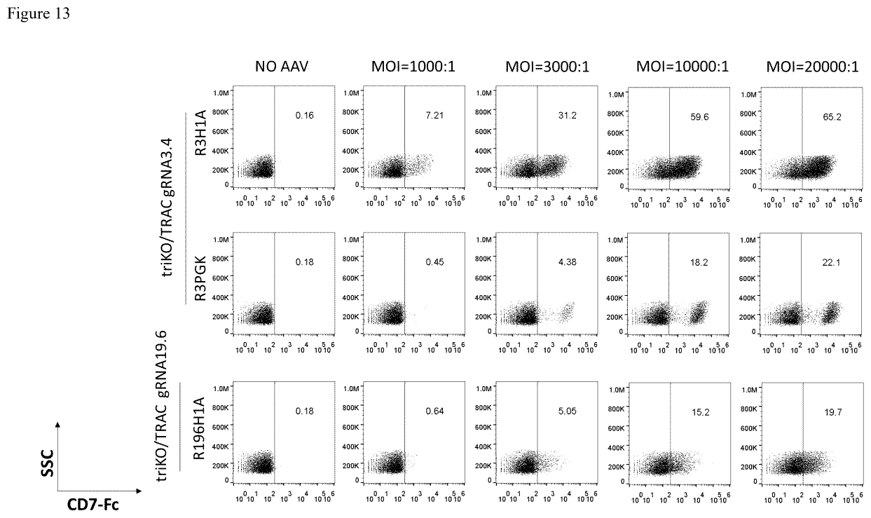

On the 8th day after electroporation, the above test was repeated, and the results showed that the knockout efficiency of the three genes (TCR/B2M/CD7) remained unchanged (FIGS. 11 and 12). At the same time, the expression of CD7 CAR and A40C2828 (LACO-stim) also remained constant (FIGS. 13 and 14).

As shown in the flow cytometry analysis in FIG. 10, the percentage of CD7-positive cells decreased to 0%. This demonstrates that the TriKO CD7 CAR-T cells prepared using this method can completely eliminate CD7-positive T cells.

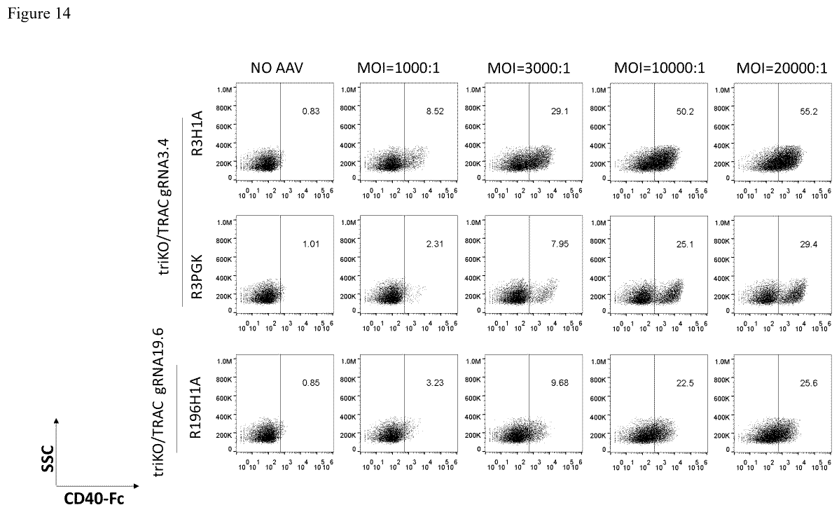

The cell growth curve demonstrated that higher AAV MOI resulted in increased CAR positivity rates but concurrently reduced CAR-T cell expansion kinetics (FIG. 15).

### Example 9: In Vitro Cytotoxicity Assay for TriKO CD7 CAR-T Cell Preparation

1. Twelve hours before the co-culture of the TriKO CD7 CAR-T cells prepared in Example 8 and the tumor target cells cytotoxicity experiment, the CCRF-CEM-GFP cells were inoculated into Poly-L-Ornithine (Sigma-Aldrich, Cat. No. P4957), 10,000 cells/100 μL per well of a pre-coated flat-bottom 96-well plate.
2. After 12 hours, when the CCRF-CEM-GFP cells were fully attached to the wall, the different TriKO CD7 CAR-T cells prepared in Example 8 were diluted to an appropriate cell density, and then co-incubated with tumor cells at different effector-target ratios (such as E:T=2:1, 0.5:1 and 0.2:1).
3. The 96-well plates were placed into the Incucyte S3 machine and the scanning parameters were set.
4. After 4 days of scanning, the total green fluorescence cumulative intensity (GCU×μm²/well) was analyzed to calculate the tumor cell killing efficiency.

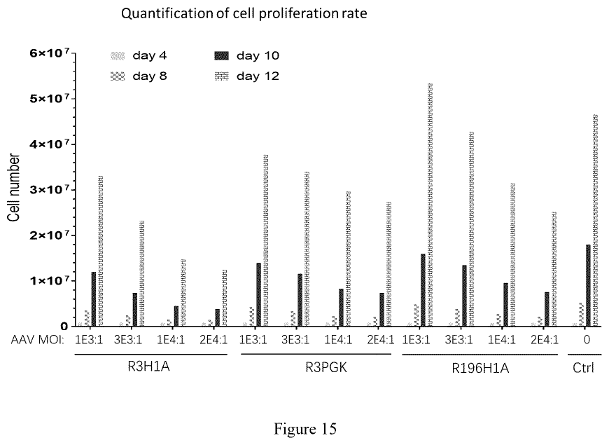

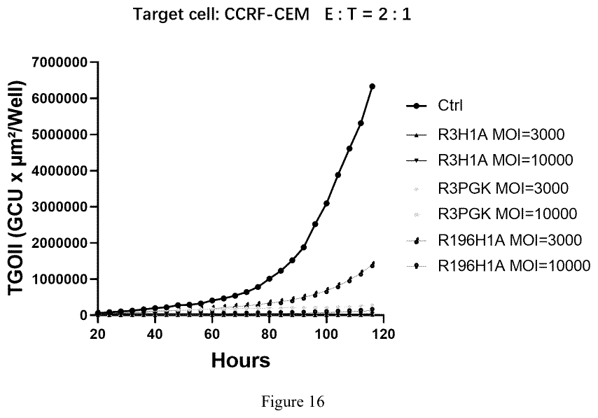

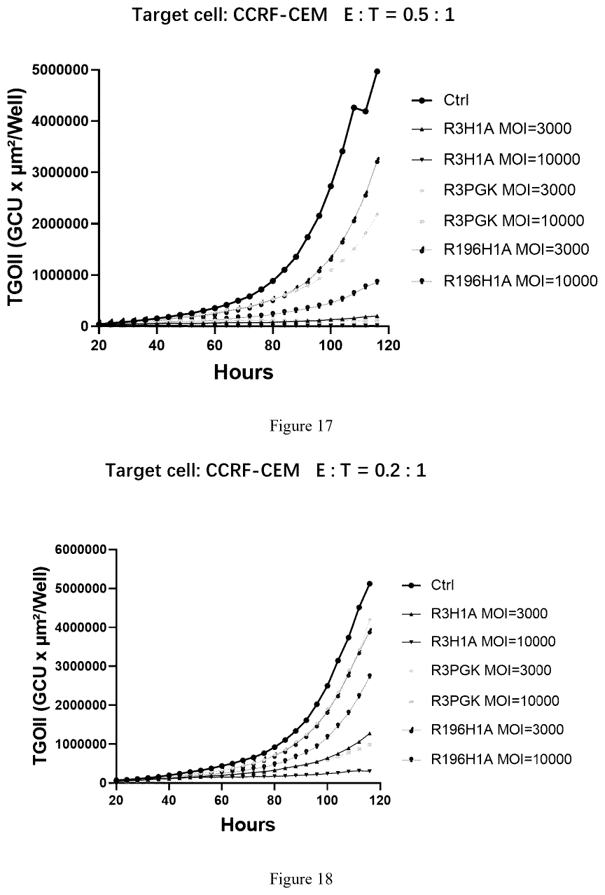

The results showed that the killing effect of the TriKO CD7 CAR-T cells prepared by the three AAVs on the CD7-positive tumor target cells CCRF-CEM was very strong, and the target cells could be effectively killed under the conditions of E:T ratio=2:1, E:T ratio=0.5:1, and even E:T ratio=0.2:1 (FIG. 16, FIG. 17 and FIG. 18). Compared with the TriKO CD7 CAR-T cells prepared by the three AAVs, the killing effect of R3H1A was stronger.

Specific sequences used in Examples 1-9:

| SEQ ID NO: | [symbol] | [symbol] |
|---|---|---|
| 1 | B2M gRNA-1 | ACTCACGCTGGATAGCCTCC |
| 2 | B2M gRNA-2 | AGTCACATGGTTCACACGGC |
| 3 | B2M gRNA-3 | GGCCGAGATGTCTCGCTCCG |
| 4 | B2M gRNA-4 | GCATACTCATCTTTTTCAGT |
| 5 | B2M gRNA-5 | TCACGTCATCCAGCAGAGAA |
| 6 | B2M gRNA-6 | GTCTTTTCCCGATATTCCTC |
| 7 | B2M gRNA-7 | ACTTGTCTTTCAGCAAGGAC |
| 8 | B2M gRNA-8 | AGGGTAGGAGAGACTCACGC |
| 9 | B2M gRNA-9 | TTCAGACTTGTCTTTCAGCA |
| 10 | B2M gRNA-10 | TTACCCCACTTAACTATCTT |
| 11 | B2M gRNA-11 | CTCAGGTACTCCAAAGATTC |
| 12 | B2M gRNA-12 | GCTACTCTCTCTTTCTGGCC |
| 13 | B2M gRNA-13 | TGGGCTGTGACAAAGTCACA |
| 14 | B2M gRNA-14 | ACAAAGTCACATGGTTCACA |
| 15 | B2M gRNA-15 | GAAGTTGACTTACTGAAGAA |
| 16 | B2M gRNA-16 | TTTGACTTTCCATTCTCTGC |
| 17 | B2M gRNA-17 | ACTCTCTCTTTCTGGCCTGG |
| 18 | B2M gRNA-18 | TGGAGAGAGAATTGAAAAAG |
| 19 | B2M gRNA-19 | TCCTGAATTGCTATGTGTCT |
| 20 | B2M gRNA-20 | GGCATACTCATCTTTTTCAG |
| 21 | B2M gRNA-21 | TCCTGAATTGCTATGTGTCT |
| 22 | B2M gRNA-22 | GAGTAGCGCGAGCACAGCTA |
| 23 | TRAC gRNA-3.4 | CGTCATGAGCAGATTAAACC |
| 24 | TRAC gRNA-19.6 | GAGAATCAAAATCGGTGAAT |
| 25 | CD7 gRNA-11 | GGGACCCTGAGAAGTCGATG |

pDA-T7-B2M gRNA 1 (pDA B2M gRNA-M1)
SEQ ID NO: 26
cccgactggaaagcgggcagtgagcgcaacgcaattaatgtgagttagctcactcattaggcaccccaggctttacactttatgcttccg
gctcgtatgttgtgtggaattgtgagcggataacaatttcacacaggaaacagctatgaccatgattacgccaagctctaatacgactcac
tatagACTCACGCTGGATAGCCTCCGTTTTAGAGCTAGAAATAGCAAGTTAAAATAAGGCTAGTC
CGTTATCAACTTGAAAAAGTGGCACCGAGTCGGTGCTTTTTTTgaattcgagctcggtacctcgcgaatgc
atctagatatcggatcccgggcccgtcgactgcagaggcctgcatgcaagcttctagtggcgcctgatgcggtattttctccttacgcatct
gtgcggtatttcacaccgcataggccgctgtattctatagtgtcacctaaatggccgcacaattcactggccgtcgttttacaacgtcgtgac
tgggaaaaccctggcgttacccaacttaatcgccttgcagcacatccccctttcgccagctggcgtaatagcgaagaggcccgcaccg
atcgcccttcccaacagttgcgcagcctgaatggcgaatggaaattgtaagcgttaatattttgttaaaattcgcgttaaatttttgttaaatca
gctcattttttaaccaataggccgaaatcggcaaaatcccttataaatcaaaagaatagaccgagatagggttgagtgttgttccagtttgg
aacaagagtccactattaaagaacgtggactccaacgtcaaagggcgaaaaaccgtctatcagggcgatggcccactacgtgaacc
atcaccctaatcaagttttttggggtcgaggtgccgtaaagcactaaatcggaaccctaaagggagcccccgatttagagcttgacggg
gaaagccggcgaacgtggcgagaaaggaagggaagaaagcgaaaggagcgggcgctagggcgctggcaagtgtagcggtcac
gctgcgcgtaaccaccacacccgccgcgcttaatgcgccgctacagggcgcgtcaggtggcacttttcggggaaatgtgcgcggaac
ccctatttgtttatttttctaaatacattcaaatatgtatccgctcatgagtcaggcaactatggatgaacgaaatagacagatcgctgagata
ggtgcctcactgattaagcattggtaactgtcagaccaagtttactcatatatactttagattgatttaaaacttcatttttaatttaaaaggatct
aggtgaagatcctttttgataatctcatgaacaataaaactgtctgcttacataaacagtaatacaaggggtgttatgagccatattcaacg
ggaaacgtcttgctctaggccgcgattaaattccaacatggatgctgatttatatgggtataaatgggctcgcgataatgtcgggcaatca
ggtgcgacaatctatcgattgtatgggaagcccgatgcgccagagttgtttctgaaacatggcaaaggtagcgttgccaatgatgttaca
gatgagatggtcagactaaactggctgacggaatttatgcctcttccgaccatcaagcattttatccgtactcctgatgatgcatggttactc
accactgcgatccccgggaaaacagcattccaggtattagaagaatatcctgattcaggtgaaaatattgttgatgcgctggcagtgttcc
tgcgccggttgcattcgattcctgtttgtaattgtccttttaacagcgatcgcgtatttcgtctcgctcaggcgcaatcacgaatgaataacggt
ttggttgatgcgagtgattttgatgacgagcgtaatggctggcctgttgaacaagtctggaaagaaatgcataaacttttgccattctcaccg
gattcagtcgtcactcatggtgatttctcacttgataaccttatttttgacgaggggaaattaataggttgtattgatgttggacgagtcggaatc
gcagaccgataccaggatcttgccatcctatggaactgcctcggtgagttttctccttcattacagaaacggctttttcaaaaatatggtattg
ataatcctgatatgaataaattgcagtttcatttgatgctcgatgagtttttctaagaattaattcatgaccaaaatcccttaacgtgagttttcgtt
ccactgagcgtcagaccccgtagaaaagatcaaaggatcttcttgagatcctttttttctgcgcgtaatctgctgcttgcaaacaaaaaaa
ccaccgctaccagcggtggtttgtttgccggatcaagagctaccaactctttttccgaaggtaactggcttcagcagagcgcagatacca
aatactgtccttctagtgtagccgtagttaggccaccacttcaagaactctgtagcaccgcctacatacctcgctctgctaatcctgttacca
gtggctgctgccagtggcgataagtcgtgtcttaccgggttggactcaagacgatagttaccggataaggcgcagcggtcgggctgaac
ggggggttcgtgcacacagcccagcttggagcgaacgacctacaccgaactgagatacctacagcgtgagctatgagaaagcgcca
cgcttcccgaagggagaaaggcggacaggtatccggtaagcggcagggtcggaacaggagagcgcacgagggagcttccaggg
ggaaacgcctggtatctttatagtcctgtcgggtttcgccacctctgacttgagcgtcgatttttgtgatgctcgtcaggggggcggagcctat
ggaaaaacgccagcaacgcggcctttttacggttcctggccttttgctggccttttgctcacatgttctttcctgcgttatcccctgattctgtgga
taaccgtattaccgcctttgagtgagctgataccgctcgccgcagccgaacgaccgagcgcagcgagtcagtgagcgaggaagcgg
aagagcgcccaatacgcaaaccgcctctccccgcgcgttggccgattcattaatgcagctggcacgacaggttt
T7 promoter
SEQ ID NO: 27
taatacgactcactatag
gRNA scaffold
SEQ ID NO: 28
GTTTTAGAGCTAGAAATAGCAAGTTAAAATAAGGCTAGTCCGTTATCAACTTGAAAAAGTGGCA
CCGAGTCGGTGC
Termination signal
SEQ ID NO: 29
TTTTTTT
Forward primer for PCR cloning
SEQ ID NO: 30
tgcagctggcacgacaggtttc
Reverse primer for PCR cloning
SEQ ID NO: 31
cgtcagactgtagcacgtt
Forward primer for Sanger sequencing
SEQ ID NO: 32
aacaattgaattcaggagga
Reverse primer for Sanger sequencing
SEQ ID NO: 33
cctcctaagaagcgtagtc
anti-CD7 scFv H1, amino acid sequence
SEQ ID NO: 34
QPVLTQSPSASETPGQRVTISCSGSTSNTGNNYFSWYQQVPGMAPKLLIYKNNLRPSGVPDRFS
GSKSGTSASLSISGLRSEDEGDYYCATWDDNLTLVFGGGTKLTVLGGGGSGGGGGGGGSQV
QLQESGGGLVQPGGSLRLSCAASGFTFSSYAMSWVRQAPGKGLEWVSAISGSGGSTYYADSV
KGRFTISRDNSKNTLYLQMNSLRAEDTAVYYCAKGYSGYPAVLDYWGLGTLVTVSS
anti-CD7 scFv H1, VL, amino acid sequence
SEQ ID NO: 35
QPVLTQSPSASETPGQRVTISCSGSTSNTGNNYFSWYQQVPGMAPKLLIYKNNLRPSGVPDRFS
GSKSGTSASLSISGLRSEDEGDYYCATWDDNLTLVFGGGTKLTVL
anti-CD7 scFv H1, VH, amino acid sequence
SEQ ID NO: 36
QVQLQESGGGLVQPGGSLRLSCAASGFTFSSYAMSWVRQAPGKGLEWVSAISGSGGSTYYAD
SVKGRFTISRDNSKNTLYLQMNSLRAEDTAVYYCAKGYSGYPAVLDYWGLGTLVTVSS
CD7H1.BBZ, amino acid sequence
SEQ ID NO: 37
MALPVTALLLPLALLLHAARPQPVLTQSPSASETPGQRVTISCSGSTSNTGNNYFSWYQQVPGM
APKLLIYKNNLRPSGVPDRFSGSKSGTSASLSISGLRSEDEGDYYCATWDDNLTLVFGGGTKLTV
LGGGGSGGGGSGGGGSQVQLQESGGGLVQPGGSLRLSCAASGFTFSSYAMSWVRQAPGKGL
EWVSAISGSGGSTYYADSVKGRFTISRDNSKNTLYLQMNSLRAEDTAVYYCAKGYSGYPAVLDY
WGLGTLVTVSSTTTPAPRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKFSRSAD
APAYKQGQNQLYNELNLGRREEYDVLDKRRGRDPEMGGKPRRKNPQEGLYNELQKDKMAEAY
SEIGMKGERRRGKGHDGLYQGLSTATKDTYDALHMQALPPR
CD7H1.BBZ, nucleotide sequence
SEQ ID NO: 38
atggccttaccagtgaccgccttgctcctgccgctggccttgctgctccacgccgccaggccgcagcctgtgctgactcagtcaccgtca
gcgtctgagacccccgggcagagggtcaccatctcttgttctgggagcacctccaacaccggaaacaattatttttcttggtaccaacaa
gtcccaggaatggcccccaaactcctcatttataagaacaatctgcggccctcaggggtccctgaccgattctctggctccaagtctggc
acctcagcctccctgtccatcagtggactccggtccgaggatgagggtgattattattgtgcaacatgggatgacaacttgactctggtgtt
cggcggagggaccaagctgaccgtcctaggtggtggtggttctggcggcggcggctccggaggtggtggatcccaggtgcagctgca
ggagtcggggggaggcttggtacagcctggggggtccctgagactctcctgtgcagcctctggattcacctttagcagctatgccatgag
ctgggtccgccaggctccagggaaggggctggagtgggtctcagctattagtggtagtggtggtagcacatactacgcagactccgtga
agggccggttcaccatctccagagacaattccaagaacacgctgtatctgcaaatgaacagcctgagagccgaggacacggccgtat
attactgtgcgaaagggtacagtggctaccccgcagtgcttgactactggggcctgggaaccctggtcactgtctcctcaaccacgacg
ccagcgccgcgaccaccaacaccggcgcccaccatcgcgtcgcagcccctgtccctgcgcccagaggcgtgccggccagcggcg
gggggcgcagtgcacacgagggggctggacttcgcctgtgatatctacatctgggcgcccttggccgggacttgtggggtccttctcctgt
cactggttatcaccctttactgcaaacggggcagaaagaaactcctgtatatattcaaacaaccatttatgagaccagtacaaactactc
aagaggaagatggctgtagctgccgatttccagaagaagaagaaggaggatgtgaactgagagtgaagttcagcaggagcgcaga
cgcccccgcgtacaagcagggccagaaccagctctataacgagctcaatctaggacgaagagaggagtacgacgttttggacaag
agacgtggccgggaccctgagatggggggaaagccgagaaggaagaaccctcaggaaggcctgtacaatgaactgcagaaaga
taagatggcggaggcctacagtgagattgggatgaaaggcgagcgccggaggggcaaggggcacgatggcctttaccagggtctc
agtacagccaccaaggacacctacgacgcccttcacatgcaggccctgccccctcgc
anti-CD7 scFv H4, amino acid sequence
SEQ ID NO: 39
QSWVTQPPSVSAAPGQKVTISCSGSSSNIGNNYVSWYQQLPGTAPKLLIYDNNKRPSGIPDRFS
GSKSGTSATLGITGLQTGDEADYYCGTWDSSLSAGDWVFGGGTKLTVLGGGGSGGGGSGGG
GSQVQLVQSGGGLVQPGGSLRLSCAASGFTFSTYWMSWVRQAPGKGLEWVANINQDGTEKYY
VDSVKGRFTISRDNAKNSLYLQMNSLRAEDTAVYYCAREPGIALDYWGQGTLVTVSS
anti-CD7 scFv H4, VL, amino acid sequence
SEQ ID NO: 40
QSVVTQPPSVSAAPGQKVTISCSGSSSNIGNNYVSWYQQLPGTAPKLLIYDNNKRPSGIPDRFS
GSKSGTSATLGITGLQTGDEADYYCGTWDSSLSAGDWVFGGGTKLTVL
anti-CD7 scFv H4, VH, amino acid sequence
SEQ ID NO: 41
QVQLVQSGGGLVQPGGSLRLSCAASGFTFSTYWMSWVRQAPGKGLEWVANINQDGTEKYYVD
SVKGRFTISRDNAKNSLYLQMNSLRAEDTAVYYCAREPGIALDYWGQGTLVTVSS
CD7H4.BBZ, amino acid sequence
SEQ ID NO: 42
MALPVTALLLPLALLLHAARPQSVVTQPPSVSAAPGQKVTISCSGSSSNIGNNYVSWYQQLPGTA
PKLLIYDNNKRPSGIPDRFSGSKSGTSATLGITGLQTGDEADYYCGTWDSSLSAGDWVFGGGTK
LTVLGGGGSGGGGSGGGGSQVQLVQSGGGLVQPGGSLRLSCAASGFTFSTYWMSWVRQAPG
KGLEWVANINQDGTEKYYVDSVKGRFTISRDNAKNSLYLQMNSLRAEDTAVYYCAREPGIALDY
WGQGTLVTVSSTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVKFSRSA
DAPAYKQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
anti-CD7 scFv H6, amino acid sequence
SEQ ID NO: 43
QSALTQPPSASGSPGQSVTISCTGTSSDVGGYNYVSWYQQHPGKAPKLMIYEVRKRPSGVPDR
FSGSKSGNTASLTVSGLQADDEADYYCSSYAGSNNLGFGGGTKLTVLGGGGSGGGGSGGGGS
EVQLVESGGGLVQPGGSLRLSCAASGFTFSSYAMSWVRQAPGKGLEWVSAISGSGGSTYYADS
VKGRFTISRDNSKNTLYLQMNSLRAEDTAVYYCAKDPDPGYFDWVAGDYWGQGTLVTVSS
anti-CD7 scFv H6, VL, amino acid sequence
SEQ ID NO: 44
QSALTQPPSASGSPGQSVTISCTGTSSDVGGYNYVSWYQQHPGKAPKLMIYEVRKRPSGVPDR
FSGSKSGNTASLTVSGLQADDEADYYCSSYAGSNNLGFGGGTKLTVL
anti-CD7 scFv H6, VH, amino acid sequence
SEQ ID NO: 45
EVOLVESGGGLVQPGGSLRLSCAASGFTFSSYAMSWVRQAPGKGLEWVSAISGSGGSTYYADS
VKGRFTISRDNSKNTLYLQMNSLRAEDTAVYYCAKDPDPGYFDWVAGDYWGQGTLVTVSS
CD7H6.BBZ, amino acid sequence
SEQ ID NO: 46
MALPVTALLLPLALLLHAARPQSALTQPPSASGSPGQSVTISCTGTSSDVGGYNYVSWYQQHPG
KAPKLMIYEVRKRPSGVPDRFSGSKSGNTASLTVSGLQADDEADYYCSSYAGSNNLGFGGGTKL
TVLGGGGSGGGGSGGGGSEVQLVESGGGLVQPGGSLRLSCAASGFTFSSYAMSWVRQAPGK
GLEWVSAISGSGGSTYYADSVKGRFTISRDNSKNTLYLQMNSLRAEDTAVYYCAKDPDPGYFDW
VAGDYWGQGTLVTVSSTTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELRVK
FSRSADAPAYKQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
anti-CD7 scFv H8, amino acid sequence
SEQ ID NO: 47
QSVLTQPPSVSEAPRQRVTISCSGSSSNIGNNAVNWYQQLPGKAPKLLIYYDDLLPSGVSDRFSG
SKSGTSASLTISGLQSEDGADYYCASWDDSLNGRVFGGGTKLTVLGGGGSGGGGSGGGGSQV
QLQESGAGLLKPSETLSLTCAVYGGSISGYYWSWIRQPPGKGLEWIGEINHSGSTNYNPSLKSR
VTISVDTSKNQFSLKLSSVTAADTAVYYCARGGGSSGYYLDYWGQGALVTVSS
anti-CD7 scFv H8, VL, amino acid sequence
SEQ ID NO: 48
QSVLTQPPSVSEAPRQRVTISCSGSSSNIGNNAVNWYQQLPGKAPKLLIYYDDLLPSGVSDRFSG
SKSGTSASLTISGLQSEDGADYYCASWDDSLNGRVFGGGTKLTVL
anti-CD7 scFv H8, VH, amino acid sequence
SEQ ID NO: 49
QVQLQESGAGLLKPSETLSLTCAVYGGSISGYYWSWIRQPPGKGLEWIGEINHSGSTNYNPSLK
SRVTISVDTSKNQFSLKLSSVTAADTAVYYCARGGGSSGYYLDYWGQGALVTVSS
CD7H8.BBZ, amino acid sequence
SEQ ID NO: 50
MALPVTALLLPLALLLHAARPQSVLTQPPSVSEAPRQRVTISCSGSSSNIGNNAVNWYQQLPGKA
PKLLIYYDDLLPSGVSDRFSGSKSGTSASLTISGLQSEDGADYYCASWDDSLNGRVFGGGTKLTV
LGGGGSGGGGSGGGGSQVQLQESGAGLLKPSETLSLTCAVYGGSISGYYWSWIRQPPGKGLE
WIGEINHSGSTNYNPSLKSRVTISVDTSKNQFSLKLSSVTAADTAVYYCARGGGSSGYYLDYWG
QGALVTVSSTTTPAPRPPTPAPTIASQPLSLRPEACRPAAGGAVHTRGLDFACDIYIWAPLAGTCG
VLLLSLVITLYCKRGRKKLLYIFKQPFMRPVQTTQEEDGCSCRFPEEEEGGCELRVKFSRSADAPA
YKQGQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR
anti-CD7 scFv H9, amino acid sequence
SEQ ID NO: 51
QSVLTQPPSASGAPGERVTISCSGSSSNIGSNYVYWYQQLPGTAPKLLIYNSNQRPSGVPDRFS
GSKSGASASLAISGLRSEDEADYYCAAWDDSLSGPVFGGGTKVTVLGGGGSGGGGSGGGGSE
VQLVESGAEVKKPGASVKVSCKASGYTFTGYYMHWVRQAPGQGLEWMGWINPNSGGTNYAQ
KFQGRVTMTRDTSISTAYMELSRLRSDDTAVYYCARGVGLWFGEPYFDYWGQGTLVTVSS
anti-CD7 scFv H9, VL, amino acid sequence
SEQ ID NO: 52
QSVLTQPPSASGAPGERVTISCSGSSSNIGSNYVYWYQQLPGTAPKLLIYNSNQRPSGVPDRFS
GSKSGASASLAISGLRSEDEADYYCAAWDDSLSGPVFGGGTKVTVL
anti-CD7 scFv H9, VH, amino acid sequence
SEQ ID NO: 53
EVOLVESGAEVKKPGASVKVSCKASGYTFTGYYMHWVRQAPGQGLEWMGWINPNSGGTNYA
QKFQGRVTMTRDTSISTAYMELSRLRSDDTAVYYCARGVGLWFGEPYFDYWGQGTLVTVSS
CD7H9.BBZ, amino acid sequence
SEQ ID NO: 54
MALPVTALLLPLALLLHAARPQSVLTQPPSASGAPGERVTISCSGSSSNIGSNYVYWYQQLPGTA
PKLLIYNSNQRPSGVPDRFSGSKSGASASLAISGLRSEDEADYYCAAWDDSLSGPVFGGGTKVT
VLGGGGSGGGGSGGGGSEVQLVESGAEVKKPGASVKVSCKASGYTFTGYYMHWVRQAPGQG
LEWMGWINPNSGGTNYAQKFQGRVTMTRDTSISTAYMELSRLRSDDTAVYYCARGVGLWFGEP
YFDYWGQGTLVTVSSTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRVKF
SRSADAPAYKQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
anti-CD7 scFv H10, amino acid sequence
SEQ ID NO: 55
QSALTQPASVSGSPGQSITISCTGTSSDVGGSKYVSWYQQHPGKAPKLMIYDVSYRPSGVSDRF
SGSKSGNTASLTISGLQAEDEADYYCSSYTSNSNLPLFGGGTKVTVLGGGGSGGGGSGGGGSQ
LQLQESGGGLVQPGGSLRLSCAASGFTFSSYAMSWVRQAPGKGLEWVSAISGSGGSTYYADSV
KGRFTISRDNSKNTLYLQMNSLRAEDTAVYYCAKGYSGYPAVLDYWGQGTLVTVSS
anti-CD7 scFv H10, VL, amino acid sequence
SEQ ID NO: 56
QSALTQPASVSGSPGQSITISCTGTSSDVGGSKYVSWYQQHPGKAPKLMIYDVSYRPSGVSDRF
SGSKSGNTASLTISGLQAEDEADYYCSSYTSNSNLPLFGGGTKVTVL
anti-CD7 scFv H10, VH, amino acid sequence
SEQ ID NO: 57
QLQLQESGGGLVQPGGSLRLSCAASGFTFSSYAMSWVRQAPGKGLEWVSAISGSGGSTYYAD
SVKGRFTISRDNSKNTLYLQMNSLRAEDTAVYYCAKGYSGYPAVLDYWGQGTLVTVSS
CD7H10.BBZ, amino acid sequence
SEQ ID NO: 58
MALPVTALLLPLALLLHAARPQSALTQPASVSGSPGQSITISCTGTSSDVGGSKYVSWYQQHPGK
APKLMIYDVSYRPSGVSDRFSGSKSGNTASLTISGLQAEDEADYYCSSYTSNSNLPLFGGGTKVT
VLGGGGSGGGGSGGGGSQLQLQESGGGLVQPGGSLRLSCAASGFTFSSYAMSWVRQAPGKG
LEWVSAISGSGGSTYYADSVKGRFTISRDNSKNTLYLQMNSLRAEDTAVYYCAKGYSGYPAVLDY
WGQGTLVTVSSTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVKFSRSA
DAPAYKQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
anti-CD7 scFv H12, amino acid sequence
SEQ ID NO: 59
QPVLTQPPSASGTPGQRVIISCSGSGSNIGSNTVTWYRQFPGAAPKTLIYNSKLRPSWVPDRFTA
SKSGTSASLAISGLQSEDEADYYCAAWDDSLNGVVFGGGTKLTVLGGGGSGGGGSGGGGSEV
HLVESGGGLVQPGGSLRLSCAASGFTFSSYAMSWVRQAPGKGLEWVSAISGSGGSTYYADSVK
GRFTISRDNSKNTLYLQMNSLRAEDTAVYYCAKGYSGYPAVLDYWGQGTLVTVSS
anti-CD7 scFv H12, VL, amino acid sequence
SEQ ID NO: 60
QPVLTQPPSASGTPGQRVIISCSGSGSNIGSNTVTWYRQFPGAAPKTLIYNSKLRPSWVPDRFTA
SKSGTSASLAISGLQSEDEADYYCAAWDDSLNGVVFGGGTKLTVL
anti-CD7 scFv H12, VH, amino acid sequence
SEQ ID NO: 61
EVHLVESGGGLVQPGGSLRLSCAASGFTFSSYAMSWVRQAPGKGLEWVSAISGSGGSTYYADS
VKGRFTISRDNSKNTLYLQMNSLRAEDTAVYYCAKGYSGYPAVLDYWGQGTLVTVSS
CD7H12.BBZ, amino acid sequence
SEQ ID NO: 62
MALPVTALLLPLALLLHAARPQPVLTQPPSASGTPGQRVIISCSGSGSNIGSNTVTWYRQFPGAA
PKTLIYNSKLRPSWVPDRFTASKSGTSASLAISGLQSEDEADYYCAAWDDSLNGVVFGGGTKLT
VLGGGGSGGGGSGGGGSEVHLVESGGGLVQPGGSLRLSCAASGFTFSSYAMSWVRQAPGKG
LEWVSAISGSGGSTYYADSVKGRFTISRDNSKNTLYLQMNSLRAEDTAVYYCAKGYSGYPAVLDY
WGQGTLVTVSSTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVKFSRSA
DAPAYKQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
anti-CD7 scFv H13, amino acid sequence
SEQ ID NO: 63
QSVVTQPPSASGTPGQRVTISCSGSSSNIGSNSVNWYQQLPGTAPKLLIYNNNQRPSGVPDRFS
GSKSGTSASLAISGLQSEDEADYYCATWDDSLNGNVVFGGGTKVTVLGGGGSGGGGSGGGGS
QVQLVQSGAEVKKPGSSVKVSCKASGGTFSSYAISWVRQAPGQGLEWMGGIIPIFGTANYAQKF
QGRVTITADESTSTAYMELSSLRSEDTAVYYCARDQGVVPAAEIPYYDFWSGPPFDPWGQGTLV
TVSS
anti-CD7 scFv H13, VL, amino acid sequence
SEQ ID NO: 64
QSVVTQPPSASGTPGQRVTISCSGSSSNIGSNSVNWYQQLPGTAPKLLIYNNNQRPSGVPDRFS
GSKSGTSASLAISGLQSEDEADYYCATWDDSLNGNVVFGGGTKVTVL
anti-CD7 scFv H13, VH, amino acid sequence
SEQ ID NO: 65
QVQLVQSGAEVKKPGSSVKVSCKASGGTFSSYAISWVRQAPGQGLEWMGGIIPIFGTANYAQKF
QGRVTITADESTSTAYMELSSLRSEDTAVYYCARDQGVVPAAEIPYYDFWSGPPFDPWGQGTLV
TVSS
CD7H13.BBZ, amino acid sequence
SEQ ID NO: 66
MALPVTALLLPLALLLHAARPQSVVTQPPSASGTPGQRVTISCSGSSSNIGSNSVNWYQQLPGTA
PKLLIYNNNQRPSGVPDRFSGSKSGTSASLAISGLQSEDEADYYCATWDDSLNGNVVFGGGTKV
TVLGGGGSGGGGSGGGGSQVQLVQSGAEVKKPGSSVKVSCKASGGTFSSYAISWVRQAPGQ
GLEWMGGIIPIFGTANYAQKFQGRVTITADESTSTAYMELSSLRSEDTAVYYCARDQGVVPAAEIP
YYDFWSGPPFDPWGQGTLVTVSSTTTPAPRPPTPAPTIASQPLSLRPEACRPAAGGAVHTRGLD
FACDIYIWAPLAGTCGVLLLSLVITLYCKRGRKKLLYIFKQPFMRPVQTTQEEDGCSCRFPEEEEG
GCELRVKFSRSADAPAYKQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR
anti-CD7 scFv H17, amino acid sequence
SEQ ID NO: 67
QSALTQPPSVSGSPGQSVSISCTGSSSDIGASSRVSWYLQPPGKAPQLIIFGVNNRPSGVPDRF
SGSKSGNTASLTISGLRAEDEADYYCSSHSSGNTLLFGGGTKLTVLGGGSGGGGSGGGGSQVQ
LQESGGGLVQPGGSLRLSCAASGFTFSSYAMSWVRQAPGKGLEWVSAISGSGGSTYYADSVK
GRFTISRDNSKNTLYLQMNSLRAEDTAVYYCAKGYSGYPAVLDYWGQGTLVTVSS
anti-CD7 scFv H17, VL, amino acid sequence
SEQ ID NO: 68
QSALTQPPSVSGSPGQSVSISCTGSSSDIGASSRVSWYLQPPGKAPQLIIFGVNNRPSGVPDRF
SGSKSGNTASLTISGLRAEDEADYYCSSHSSGNTLLFGGGTKLTVL
anti-CD7 scFv H17, VH, amino acid sequence
SEQ ID NO: 69
QVQLQESGGGLVQPGGSLRLSCAASGFTFSSYAMSWVRQAPGKGLEWVSAISGSGGSTYYAD
SVKGRFTISRDNSKNTLYLQMNSLRAEDTAVYYCAKGYSGYPAVLDYWGQGTLVTVSS
CD7H17.BBZ, amino acid sequence
SEQ ID NO: 70
MALPVTALLLPLALLLHAARPQSALTQPPSVSGSPGQSVSISCTGSSSDIGASSRVSWYLQPPGK
APQLIIFGVNNRPSGVPDRFSGSKSGNTASLTISGLRAEDEADYYCSSHSSGNTLLFGGGTKLTVL
GGGSGGGGSGGGGSQVQLQESGGGLVQPGGSLRLSCAASGFTFSSYAMSWVRQAPGKGLE
WVSAISGSGGSTYYADSVKGRFTISRDNSKNTLYLQMNSLRAEDTAVYYCAKGYSGYPAVLDYW
GQGTLVTVSSTTTPAPRPPTPAPTIASQPLSLRPEACRPAAGGAVHTRGLDFACDIYIWAPLAGTC
GVLLLSLVITLYCKRGRKKLLYIFKQPFMRPVQTTQEEDGCSCRFPEEEEGGCELRVKFSRSADA
PAYKQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR
anti-CD7 scFv H18, amino acid sequence
SEQ ID NO: 71
QSALTQPPSASGSPGQSVTISCTGTSSDVGGYTYVSWYQQHPGKAPKLMIYEVSKRPSGVPDR
FSGSKSGNTASLTVSGLQAEDEADYYCSSYAGSNNVVFGGGTELTVLGGGGSGGGGSGGGGS
EVQLVESGGGLVQPGGSLRLSCAASGFTFSSYAMSWVRQAPGKGLEWFSAISGSGGSTYYADS
VKGRFTISRDNSKNTLYLQMNSLRAEDTAVYYCAKDPDPGYFDWVAGDYWGQGTLVTVSS
anti-CD7 scFv H18, VL, amino acid sequence
SEQ ID NO: 72
QSALTQPPSASGSPGQSVTISCTGTSSDVGGYTYVSWYQQHPGKAPKLMIYEVSKRPSGVPDR
FSGSKSGNTASLTVSGLQAEDEADYYCSSYAGSNNVVFGGGTELTVL
anti-CD7 scFv H18, VH, amino acid sequence
SEQ ID NO: 73
EVOLVESGGGLVQPGGSLRLSCAASGFTFSSYAMSWVRQAPGKGLEWFSAISGSGGSTYYADS
VKGRFTISRDNSKNTLYLQMNSLRAEDTAVYYCAKDPDPGYFDWVAGDYWGQGTLVTVSS
CD7H18.BBZ, amino acid sequence
SEQ ID NO: 74
MALPVTALLLPLALLLHAARPQSALTQPPSASGSPGQSVTISCTGTSSDVGGYTYVSWYQQHPG
KAPKLMIYEVSKRPSGVPDRFSGSKSGNTASLTVSGLQAEDEADYYCSSYAGSNNVVFGGGTEL
TVLGGGGSGGGGSGGGGSEVQLVESGGGLVQPGGSLRLSCAASGFTFSSYAMSWVRQAPGK
GLEWFSAISGSGGSTYYADSVKGRFTISRDNSKNTLYLQMNSLRAEDTAVYYCAKDPDPGYFDW
VAGDYWGQGTLVTVSSTTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELRVK
FSRSADAPAYKQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
anti-CD7 scFv H5, amino acid sequence
SEQ ID NO: 75
DIRLTQSPSTVSASIGDRVTITCRASQGISNWLTWYQQKPGKAPKLLIYSASTLQSGVPSRFSGSG
SGTDFTLTISSLQPEDFATYYCQQANNLRTLTFGGGTKVDIKGGGGSGGGGSGGGGSQVQLVQ
SGAEVKKPGESLRISCKGSGYSFTSYWISWVRQMPGKGLEWMGRIDPSDSYTNYSPSFQGHVT
ISADKSISTAYLQWSSLKASDTAMYYCARHPVTNYYYYGMDVWGQGTTVTVSS
anti-CD7 scFv H5, VL, amino acid sequence
SEQ ID NO: 76
DIRLTQSPSTVSASIGDRVTITCRASQGISNWLTWYQQKPGKAPKLLIYSASTLQSGVPSRFSGSG
SGTDFTLTISSLQPEDFATYYCQQANNLRTLTFGGGTKVDIK
anti-CD7 scFv H5, VH, amino acid sequence
SEQ ID NO: 77
QVQLVQSGAEVKKPGESLRISCKGSGYSFTSYWISWVRQMPGKGLEWMGRIDPSDSYTNYSPS
FQGHVTISADKSISTAYLQWSSLKASDTAMYYCARHPVTNYYYYGMDVWGQGTTVTVSS
CD7H5.BBZ, amino acid sequence
SEQ ID NO: 78
MALPVTALLLPLALLLHAARPDIRLTQSPSTVSASIGDRVTITCRASQGISNWLTWYQQKPGKAPK
LLIYSASTLQSGVPSRFSGSGSGTDFTLTISSLQPEDFATYYCQQANNLRTLTFGGGTKVDIKGGG
GSGGGGSGGGGSQVQLVQSGAEVKKPGESLRISCKGSGYSFTSYWISWVRQMPGKGLEWMG
RIDPSDSYTNYSPSFQGHVTISADKSISTAYLQWSSLKASDTAMYYCARHPVTNYYYYGMDVWG
QGTTVTVSSTTTPAPRPPTPAPTIASQPLSLRPEACRPAAGGAVHTRGLDFACDIYIWAPLAGTCG
VLLLSLVITLYCKRGRKKLLYIFKQPFMRPVQTTQEEDGCSCRFPEEEEGGCELRVKFSRSADAPA
YKQGQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR
anti-CD7 scFv H7, amino acid sequence
SEQ ID NO: 79
DIVMTQSPSSLSASVGDRVTITCRASQSISSYLNWYQQKPGKAPKLLIYAASSLQSGVPSRFSGS
GSGTDFTLTISSLQPEDFATYYCQQSYSTLPYTFGQGTKLEIKGGGGSGGGGSGGGGSEVQLVE
SGGGLIQPGGSLRLSCAASGFTVSSNYMSWVRQAPGKGLEWVSVIYSGGSTYYADSVKGRFTI
SRDNSKNTLYLQMNSLRAEDTAVYYCATDYGELHDAFDIWGQGTTVTVSS
anti-CD7 scFv H7, VL, amino acid sequence
SEQ ID NO: 80
DIVMTQSPSSLSASVGDRVTITCRASQSISSYLNWYQQKPGKAPKLLIYAASSLQSGVPSRFSGS
GSGTDFTLTISSLQPEDFATYYCQQSYSTLPYTFGQGTKLEIK
anti-CD7 scFv H7, VH, amino acid sequence
SEQ ID NO: 81
EVOLVESGGGLIQPGGSLRLSCAASGFTVSSNYMSWVRQAPGKGLEWVSVIYSGGSTYYADSV
KGRFTISRDNSKNTLYLQMNSLRAEDTAVYYCATDYGELHDAFDIWGQGTTVTVSS
CD7H7.BBZ, amino acid sequence
SEQ ID NO: 82
MALPVTALLLPLALLLHAARPDIVMTQSPSSLSASVGDRVTITCRASQSISSYLNWYQQKPGKAPK
LLIYAASSLQSGVPSRFSGSGSGTDFTLTISSLQPEDFATYYCQQSYSTLPYTFGQGTKLEIKGGG
GSGGGGSGGGGSEVQLVESGGGLIQPGGSLRLSCAASGFTVSSNYMSWVRQAPGKGLEWVS
VIYSGGSTYYADSVKGRFTISRDNSKNTLYLQMNSLRAEDTAVYYCATDYGELHDAFDIWGQGTT
VTVSSTTTPAPRPPTPAPTIASQPLSLRPEACRPAAGGAVHTRGLDFACDIYIWAPLAGTCGVLLL
SLVITLYCKRGRKKLLYIFKQPFMRPVQTTQEEDGCSCRFPEEEEGGCELRVKFSRSADAPAYKQ
GQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
anti-CD7 scFv H15, amino acid sequence
SEQ ID NO: 83
AIQLTQSPSSVSASVGDRVTITCRASQGISSWLAWYQQKPGKAPKLLIYAASSLQSGVPSRFSGS
GSGTDFTLTISSLQPEDFATYYCQQANSFPYTFGQGTKVEIKGGGGGGGGSGGGGSEVQLVQ
SGAEVKKPGASVKVSCKVSGYTLTELSMHWVRQAPGKGLEWMGGFDPEDGETIYAQKFQGRV
TMTEDTSTDTAYMELSSLRSEDTAVYYCATERASGSYEYYFDYWGQGTLVTVSS
anti-CD7 scFv H15, VL, amino acid sequence
SEQ ID NO: 84
AIQLTQSPSSVSASVGDRVTITCRASQGISSWLAWYQQKPGKAPKLLIYAASSLQSGVPSRFSGS
GSGTDFTLTISSLQPEDFATYYCQQANSFPYTFGQGTKVEIK
anti-CD7 scFv H15, VH, amino acid sequence
SEQ ID NO: 85
EVQLVQSGAEVKKPGASVKVSCKVSGYTLTELSMHWVRQAPGKGLEWMGGFDPEDGETIYAQ
KFQGRVTMTEDTSTDTAYMELSSLRSEDTAVYYCATERASGSYEYYFDYWGQGTLVTVSS
CD7H15.BBZ, amino acid sequence
SEQ ID NO: 86
MALPVTALLLPLALLLHAARPAIQLTQSPSSVSASVGDRVTITCRASQGISSWLAWYQQKPGKAPK
LLIYAASSLQSGVPSRFSGSGSGTDFTLTISSLQPEDFATYYCQQANSFPYTFGQGTKVEIKGGG
GSGGGGSGGGGSEVQLVQSGAEVKKPGASVKVSCKVSGYTLTELSMHWVRQAPGKGLEWMG
GFDPEDGETIYAQKFQGRVTMTEDTSTDTAYMELSSLRSEDTAVYYCATERASGSYEYYFDYWG
QGTLVTVSSTTTPAPRPPTPAPTIASQPLSLRPEACRPAAGGAVHTRGLDFACDIYIWAPLAGTCG
VLLLSLVITLYCKRGRKKLLYIFKQPFMRPVQTTQEEDGCSCRFPEEEEGGCELRVKFSRSADAPA
YKQGQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR

| SEQ ID | | |
|---|---|---|
| NO: | [symbol] | [symbol] |
| 87 | H1 HCDR1; H6 HCDR1; | GFTFSSY |
| | H10 HCDR1; H12 HCDR1; | |
| | H17 HCDR1; H18 HCDR1 | |
| 88 | H4 HCDR1 | GFTFSTY |
| 89 | H8 HCDR1 | GGSISGY |
| 90 | H9 HCDR1 | GYTFTGY |
| 91 | H13 HCDR1 | GGTFSSY |
| 92 | H5 HCDR1 | GYSFTSY |
| 93 | H7 HCDR1 | GFTVSSN |
| 94 | H15 HCDR1 | GYTLTEL |
| 95 | H1 HCDR2; H6 HCDR2; | SGSGGS |
| | H10 HCDR2; H12 HCDR2; | |
| | H17 HCDR2; H18 HCDR2 | |
| 96 | H4 HCDR2 | NQDGTE |
| 97 | H8 HCDR2 | NHSGS |
| 98 | H9 HCDR2 | NPNSGG |
| 99 | H13 HCDR2 | IPIFGT |
| 100 | H5 HCDR2 | DPSDSY |
| 101 | H7 HCDR2 | YSGGS |
| 102 | H15 HCDR2 | DPEDGE |
| 103 | H1 HCDR3; H10 HCDR3; | GYSGYPAVLDY |
| | H12 HCDR3; H17 HCDR3 | |
| 104 | H4 HCDR3 | EPGIALDY |
| 105 | H6 HCDR3 | DPDPGYFDWVAGDY |
| 106 | H8 HCDR3 | GGGSSGYYLDY |
| 107 | H9 HCDR3 | GVGLWFGEPYFDY |
| 108 | H13 HCDR3 | DQGVVPAAEIPYYD |
| | | FWSGPPFDP |
| 109 | H18 HCDR3 | KDPDPGYFDWVAGDY |
| 110 | H5 HCDR3 | HPVTNYYYYGMDV |
| 111 | H7 HCDR3 | DYGELHDAFDI |
| 112 | H15 HCDR3 | ERASGSYEYYFDY |
| 113 | H1 LCDR1 | SGSTSNTGNNYFS |
| 114 | H4 LCDR1 | SGSSSNIGNNYVS |
| 115 | H6 LCDR1 | TGTSSDVGGYNYVS |
| 116 | H8 LCDR1 | SGSSSNIGNNAVN |
| 117 | H9 LCDR1 | SGSSSNIGSNYVY |
| 118 | H10 LCDR1 | TGTSSDVGGSKYVS |
| 119 | H12 LCDR1 | SGSGSNIGSNTVT |
| 120 | H13 LCDR1 | SGSSSNIGSNSVN |
| 121 | H17 LCDR1 | TGSSSDIGASSRVS |
| 122 | H18 LCDR1 | TGTSSDVGGYTYVS |
| 123 | H5 LCDR1 | RASQGISNWLT |
| 124 | H7 LCDR1 | RASQSISSYLN |
| 125 | H15 LCDR1 | RASQGISSWLA |
| 126 | H1 LCDR2 | KNNLRPS |
| 127 | H4 LCDR2 | DNNKRPS |
| 128 | H6 LCDR2 | EVRKRPS |
| 129 | H8 LCDR2 | YDDLLPS |
| 130 | H9 LCDR2 | NSNQRPS |
| 131 | H10 LCDR2 | DVSYRPS |
| 132 | H12 LCDR2 | NSKLRPS |
| 133 | H13 LCDR2 | NNNQRPS |
| 134 | H17 LCDR2 | GVNNRPS |
| 135 | H18 LCDR2 | EVSKRPS |
| 136 | H5 LCDR2 | SASTLQS |
| 137 | H7 LCDR2; H15 LCDR2 | AASSLQS |
| 138 | H1 LCDR3 | ATWDDNLTLV |
| 139 | H4 LCDR3 | GTWDSSLSAGDWV |
| 140 | H6 LCDR3 | SSYAGSNNLG |
| 141 | H8 LCDR3 | ASWDDSLNGRV |
| 142 | H9 LCDR3 | AAWDDSLSGPV |
| 143 | H10 LCDR3 | SSYTSNSNLPL |
| 144 | H12 LCDR3 | AAWDDSLNGVV |
| 145 | H13 LCDR3 | ATWDDSLNGNVV |
| 146 | H17 LCDR3 | SSHSSGNTLL |
| 147 | H18 LCDR3 | SSYAGSNNVV |
| 148 | H5 LCDR3 | QQANNLRTLT |
| 149 | H7 LCDR3 | QQSYSTLPYT |
| 150 | H15 LCDR3 | QQANSFPYT |

CD8 signal peptide, amino acid sequence
SEQ ID NO: 151
MALPVTALLLPLALLLHAARP
CD8 signal peptide, nucleotide sequence
SEQ ID NO: 152
atggccttaccagtgaccgccttgctcctgccgctggccttgctgctccacgccgccaggccg
CD8 hinge region, amino acid sequence
SEQ ID NO: 153
TTTPAPRPPTPAPTIASQPLSLRPEACRPAAGGAVHTRGLDFACD
CD8 hinge region, nucleotide sequence
SEQ ID NO: 154
accacgacgccagcgccgcgaccaccaacaccggcgcccaccatcgcgtcgcagcccctgtccctgcgcccagaggcgtgccgg
ccagcggcggggggcgcagtgcacacgagggggctggacttcgcctgtgat
CD8 transmembrane domain, amino acid sequence
SEQ ID NO: 155
IYIWAPLAGTCGVLLLSLVITLYC
CD8 transmembrane domain, nucleotide sequence
SEQ ID NO: 156
atctacatctgggcgcccttggccgggacttgtggggtccttctcctgtcactggttatcaccctttactgc
4-1BB co-stimulatory domain, amino acid sequence
SEQ ID NO: 157
KRGRKKLLYIFKQPFMRPVQTTQEEDGCSCRFPEEEEGGCEL
4-1BB co-stimulatory domain, nucleotide sequence
SEQ ID NO: 158
aaacggggcagaaagaaactcctgtatatattcaaacaaccatttatgagaccagtacaaactactcaagaggaagatggctgtagct
gccgatttccagaagaagaagaaggaggatgtgaactg
CD3-zeta, amino acid sequence
SEQ ID NO: 159
RVKFSRSADAPAYKQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
CD3-zeta, nucleotide sequence
SEQ ID NO: 160
Agagtgaagttcagcaggagcgcagacgcccccgcgtacaagcagggccagaaccagctctataacgagctcaatctaggacga
agagaggagtacgacgttttggacaagagacgtggccgggaccctgagatggggggaaagccgagaaggaagaaccctcagga
aggcctgtacaatgaactgcagaaagataagatggcggaggcctacagtgagattgggatgaaaggcgagcgccggaggggcaa
ggggcacgatggcctttaccagggtctcagtacagccaccaaggacacctacgacgcccttcacatgcaggccctgccccctcgc
A40C2828, amino acid sequence
SEQ ID NO: 161
MALPVTALLLPLALLLHAARPQVQLVQSGAEVKKPGASVKVSCTASGFNIKDYYVHWVKQAPGQ
GLEWMGRIDPEDGDSKYAPKFQGKATMTADTSTSTVYMELSSLRSEDTAVYYCTTSYYVGTYGY
WGQGTLVTVSSGGGGSGGGGSGGGGSDIQMTQSPSSLSASVGDRVTITCSASSSVSYMLWFQ
QKPGKAPKLLIYSTSNLASGVPSRFSGSGSGTDFTLTISSLQPEDFATYYCQQRTFYPYTFGGGT
KVEIKRTASIEVMYPPPYLDNEKSNGTIIHVKGKHLCPSPLFPGPSKPFWVLVVVGGVLACYSLLV
TVAFIIFWVRSKRSRLLHSDYMNMTPRRPGPTRKHYQPYAPPRDFAAYRS
A40C2828, Nucleotide sequence
SEQ ID NO: 162
Atggccttaccagtgaccgccttgctcctgccgctggccttgctgctccacgccgccaggccgcaggtccagctggtacaatctggtgct
gaagtgaagaaaccaggagcgtcggtgaaggtctcatgcacagcctcaggcttcaacatcaaggactactacgtgcactgggtcaag
caagctcccggacaggggctggagtggatggggagaattgacccggaagacggcgactcgaaatacgcgccgaagttccagggc
aaggccaccatgacagccgataccagtacatcgaccgtttacatggagctgtccagcctccgctcagaggataccgcggtatactact
gtacgacctcctactacgtcggaacgtacggttactggggccagggtaccctggtgacagtgtcctcggggggcggcgggtctggcgg
gggaggcagcgggggcggcgggtcggacatccagatgactcaatccccctcttccctgagtgcgtccgtgggtgacagggtgacgat
cacttgtagcgcttcgtcctcggtgtcgtatatgctgtggttccagcagaagcccggtaaggcccccaaactgctcatttattccaccagca
acctcgccagtggcgtacccagtcgcttctccggtagtggctccggtaccgatttcacactgaccatctccagtctgcaacctgaagacttc
gcaacgtactattgtcagcagcgcacattctatccctatacgtttggggggggaccaaggtcgagatcaagcgcacagctagcattga
agttatgtatcctcctccttacctagacaatgagaagagcaatggaaccattatccatgtgaaagggaaacacctttgtccaagtccccta
tttcccggaccttctaagcccttttgggtgctggtggtggttggtggagtcctggcttgctatagcttgctagtaacagtggcctttattattttctg
ggtgaggagtaagaggagcaggctcctgcacagtgactacatgaacatgactccccgccgccccgggcccacccgcaagcattacc
agccctatgccccaccacgcgacttcgcagcctatcgctcctaa
Anti-CD40 scFv A40, amino acid sequence
SEQ ID NO: 163
QVQLVQSGAEVKKPGASVKVSCTASGFNIKDYYVHWVKQAPGQGLEWMGRIDPEDGDSKYAPK
FQGKATMTADTSTSTVYMELSSLRSEDTAVYYCTTSYYVGTYGYWGQGTLVTVSSGGGGSGGG
GSGGGGSDIQMTQSPSSLSASVGDRVTITCSASSSVSYMLWFQQKPGKAPKLLIYSTSNLASGV
PSRFSGSGSGTDFTLTISSLQPEDFATYYCQQRTFYPYTFGGGTKVEIKRTAS
CD28 hinge region, amino acid sequence
SEQ ID NO: 164
IEVMYPPPYLDNEKSNGTIIHVKGKHLCPSPLFPGPSKP
CD28 transmembrane domain, amino acid sequence
SEQ ID NO: 165
FWVLVVVGGVLACYSLLVTVAFIIFWV
CD28 intracellular domain, amino acid sequence
SEQ ID NO: 166
RSKRSRLLHSDYMNMTPRRPGPTRKHYQPYAPPRDFAAYRS
R196H1A (pAAV-HA gRNA19.6-CD7H1.BBZ-F2A-A40C2828)
SEQ ID NO: 167
cctgcaggcagctgcgcgctcgctcgctcactgaggccgcccgggcgtcgggcgacctttggtcgcccggcctcagtgagcgagcga
gcgcgcagagagggagtggccaactccatcactaggggttcctgcggccgcaacgcgtagatcctattaaataaaagaataagcagt
attattaagtagccctgcatttcaggtttccttgagtggcaggccaggcctggccgtgaacgttcactgaaatcatggcctcttggccaaga
ttgatagcttgtgcctgtccctgagtcccagtccatcacgagcagctggtttctaagatgctatttcccgtataaagcatgagaccgtgacttg
ccagccccacagagccccgcccttgtccatcactggcatctggactccagcctgggttggggcaaagagggaaatgagatcatgtcct
aaccctgatcctcttgtcccacagatatccagaaccctgaccctgccgtgtaccagctgagagactctaaatccagtgacaagtctgtctg
cctatttctagagcgcaagcgccgcagcggcagcggcgccaccaacttctccctgctgaagcaggccggcgacgtggaggagaacc
ccggccccaagcttatggccttaccagtgaccgccttgctcctgccgctggccttgctgctccacgccgccaggccgcagcctgtgctga
ctcagtcaccgtcagcgtctgagacccccgggcagagggtcaccatctcttgttctgggagcacctccaacaccggaaacaattatttttc
ttggtaccaacaagtcccaggaatggcccccaaactcctcatttataagaacaatctgcggccctcaggggtccctgaccgattctctgg
ctccaagtctggcacctcagcctccctgtccatcagtggactccggtccgaggatgagggtgattattattgtgcaacatgggatgacaac
ttgactctggtgttcggcggagggaccaagctgaccgtcctaggtggtggtggttctggcggcggcggctccggaggtggtggatccca
ggtgcagctgcaggagtcggggggaggcttggtacagcctggggggtccctgagactctcctgtgcagcctctggattcacctttagca
gctatgccatgagctgggtccgccaggctccagggaaggggctggagtgggtctcagctattagtggtagtggtggtagcacatactac
gcagactccgtgaagggccggttcaccatctccagagacaattccaagaacacgctgtatctgcaaatgaacagcctgagagccgag
gacacggccgtatattactgtgcgaaagggtacagtggctaccccgcagtgcttgactactggggcctgggaaccctggtcactgtctcc
tcaaccacgacgccagcgccgcgaccaccaacaccggcgcccaccatcgcgtcgcagcccctgtccctgcgcccagaggcgtgcc
ggccagcggcggggggcgcagtgcacacgagggggctggacttcgcctgtgatatctacatctgggcgcccttggccgggacttgtgg
ggtccttctcctgtcactggttatcaccctttactgcaaacggggcagaaagaaactcctgtatatattcaaacaaccatttatgagaccagt
acaaactactcaagaggaagatggctgtagctgccgatttccagaagaagaagaaggaggatgtgaactgagagtgaagttcagca
ggagcgcagacgcccccgcgtacaagcagggccagaaccagctctataacgagctcaatctaggacgaagagaggagtacgacg
ttttggacaagagacgtggccgggaccctgagatggggggaaagccgagaaggaagaaccctcaggaaggcctgtacaatgaact
gcagaaagataagatggcggaggcctacagtgagattgggatgaaaggcgagcgccggaggggcaaggggcacgatggccttta
ccagggtctcagtacagccaccaaggacacctacgacgcccttcacatgcaggccctgccccctcgcgtgaaacagactttgaattttg
accttctcaagttggcgggagacgtggagtccaacccagggccgatggccttaccagtgaccgccttgctcctgccgctggccttgctgc
tccacgccgccaggccgcaggtccagctggtacaatctggtgctgaagtgaagaaaccaggagcgtcggtgaaggtctcatgcacag
cctcaggcttcaacatcaaggactactacgtgcactgggtcaagcaagctcccggacaggggctggagtggatggggagaattgacc
cggaagacggcgactcgaaatacgcgccgaagttccagggcaaggccaccatgacagccgataccagtacatcgaccgtttacatg
gagctgtccagcctccgctcagaggataccgcggtatactactgtacgacctcctactacgtcggaacgtacggttactggggccaggg
taccctggtgacagtgtcctcggggggcggcgggtctggcgggggaggcagcgggggcggcgggtcggacatccagatgactcaat
ccccctcttccctgagtgcgtccgtgggtgacagggtgacgatcacttgtagcgcttcgtcctcggtgtcgtatatgctgtggttccagcaga
agcccggtaaggcccccaaactgctcatttattccaccagcaacctcgccagtggcgtacccagtcgcttctccggtagtggctccggta
ccgatttcacactgaccatctccagtctgcaacctgaagacttcgcaacgtactattgtcagcagcgcacattctatccctatacgtttgggg
gcgggaccaaggtcgagatcaagcgcacagctagcattgaagttatgtatcctcctccttacctagacaatgagaagagcaatggaac
cattatccatgtgaaagggaaacacctttgtccaagtcccctatttcccggaccttctaagcccttttgggtgctggtggtggttggtggagtc
ctggcttgctatagcttgctagtaacagtggcctttattattttctgggtgaggagtaagaggagcaggctcctgcacagtgactacatgaa
catgactccccgccgccccgggcccacccgcaagcattaccagccctatgccccaccacgcgacttcgcagcctatcgctcctaagg
atccaagcctcgactgtgccttctagttgccagccatctgttgtttgcccctcccccgtgccttccttgaccctggaaggtgccactcccactg
tcctttcctaataaaatgaggaaattgcatcgcattgtctgagtaggtgtcattctattctggggggtggggggggcaggacagcaaggg
ggaggattgggaagacaatagcaggcatgctggggatgcggtgggctgaattcactagtcaccgattttgaaaacaaatgtgtcacaa
agtaaggattctgatgtgtatatcacagacaaaactgtgctagacatgaggtctatggacttcaagagcaacagtgctgtggcctggagc
aacaaatctgactttgcatgtgcaaacgccttcaacaacagcattattccagaagacaccttcttccccagcccaggtaagggcagcttt
ggtgccttcgcaggctgtttccttgcttcaggaatggccaggttctgcccagagctctggtcaatgatgtctaaaactcctctgattggtggtct
cggccttatccattgccaccaaaaccctctttttactaagaaacagtgagccttgttctggcagtccagagaatgacacgggaaaaaagc
acacgtgcaactcaggggcctccaaaggtgcggaccgagcggccgcaggaacccctagtgatggagttggccactccctctctgcgc
gctcgctcgctcactgaggccgggcgaccaaaggtcgcccgacgcccgggctttgcccgggcggcctcagtgagcgagcgagcgc
gcagctgcctgcagg
R3H1A (pAAV-HA gRNA3.4-CD7H1.BBZ-F2A-A40C2828)
SEQ ID NO: 168
cctgcaggcagctgcgcgctcgctcgctcactgaggccgcccgggcgtcgggcgacctttggtcgcccggcctcagtgagcgagcga
gcgcgcagagagggagtggccaactccatcactaggggttcctgcggccgcaacgcgtagatcctattaaataaaaggtcctgtctctc
aagaatcccctgccactcctcacacccaccctgggcccatattcatttccatttgagttgttcttattgagtcatccttcctgtggtagcggaac
tcactaaggggcccatctggacccgaggtattgtgatgataaattctgagcacctaccccatccccagaagggctcagaaataaaata
agagccaagtctagtcggtgtttcctgtcttgaaacacaatactgttggccctggaagaatgcacagaatctgtttgtaaggggatatgca
cagaagctgcaagggacaggaggtgcaggagctgcaggcctcccccacccagcctgctctgccttggggaaaaccgtgggtgtgtc
ctgcaggccatgcaggcctgggacatgcaagcccataaccgctgtggcctcttggttttacagatacgaacctaaactttcaaaacctgt
cagtgattgggttccgaatcctcctcctgaaagtggccaagcttggctctggagccaccaacttctccctgctgaagcaggccggcgacg
tggaggagaacccaggcccatctagaatggccttaccagtgaccgccttgctcctgccgctggccttgctgctccacgccgccaggccg
cagcctgtgctgactcagtcaccgtcagcgtctgagacccccgggcagagggtcaccatctcttgttctgggagcacctccaacaccgg
aaacaattatttttcttggtaccaacaagtcccaggaatggcccccaaactcctcatttataagaacaatctgcggccctcaggggtccct
gaccgattctctggctccaagtctggcacctcagcctccctgtccatcagtggactccggtccgaggatgagggtgattattattgtgcaac
atgggatgacaacttgactctggtgttcggcggagggaccaagctgaccgtcctaggtggtggtggttctggcggcggcggctccggag
gtggtggatcccaggtgcagctgcaggagtcggggggaggcttggtacagcctggggggtccctgagactctcctgtgcagcctctgg
attcacctttagcagctatgccatgagctgggtccgccaggctccagggaaggggctggagtgggtctcagctattagtggtagtggtggt
agcacatactacgcagactccgtgaagggccggttcaccatctccagagacaattccaagaacacgctgtatctgcaaatgaacagc
ctgagagccgaggacacggccgtatattactgtgcgaaagggtacagtggctaccccgcagtgcttgactactggggcctgggaaccc
tggtcactgtctcctcaaccacgacgccagcgccgcgaccaccaacaccggcgcccaccatcgcgtcgcagcccctgtccctgcgcc
cagaggcgtgccggccagcggcggggggcgcagtgcacacgagggggctggacttcgcctgtgatatctacatctgggcgcccttgg
ccgggacttgtggggtccttctcctgtcactggttatcaccctttactgcaaacggggcagaaagaaactcctgtatatattcaaacaacca
tttatgagaccagtacaaactactcaagaggaagatggctgtagctgccgatttccagaagaagaagaaggaggatgtgaactgaga
gtgaagttcagcaggagcgcagacgcccccgcgtacaagcagggccagaaccagctctataacgagctcaatctaggacgaagag
aggagtacgacgttttggacaagagacgtggccgggaccctgagatggggggaaagccgagaaggaagaaccctcaggaaggc
ctgtacaatgaactgcagaaagataagatggcggaggcctacagtgagattgggatgaaaggcgagcgccggaggggcaagggg
cacgatggcctttaccagggtctcagtacagccaccaaggacacctacgacgcccttcacatgcaggccctgccccctcgcgtgaaac
agactttgaattttgaccttctcaagttggcgggagacgtggagtccaacccagggccgatggccttaccagtgaccgccttgctcctgcc
gctggccttgctgctccacgccgccaggccgcaggtccagctggtacaatctggtgctgaagtgaagaaaccaggagcgtcggtgaa
ggtctcatgcacagcctcaggcttcaacatcaaggactactacgtgcactgggtcaagcaagctcccggacaggggctggagtggatg
gggagaattgacccggaagacggcgactcgaaatacgcgccgaagttccagggcaaggccaccatgacagccgataccagtacat
cgaccgtttacatggagctgtccagcctccgctcagaggataccgcggtatactactgtacgacctcctactacgtcggaacgtacggtt
actggggccagggtaccctggtgacagtgtcctcggggggcggcgggtctggcgggggaggcagcgggggcggcgggtcggacat
ccagatgactcaatccccctcttccctgagtgcgtccgtgggtgacagggtgacgatcacttgtagcgcttcgtcctcggtgtcgtatatgct
gtggttccagcagaagcccggtaaggcccccaaactgctcatttattccaccagcaacctcgccagtggcgtacccagtcgcttctccg
gtagtggctccggtaccgatttcacactgaccatctccagtctgcaacctgaagacttcgcaacgtactattgtcagcagcgcacattctat
ccctatacgtttggggggggaccaaggtcgagatcaagcgcacagctagcattgaagttatgtatcctcctccttacctagacaatgag
aagagcaatggaaccattatccatgtgaaagggaaacacctttgtccaagtcccctatttcccggaccttctaagcccttttgggtgctggt
ggtggttggtggagtcctggcttgctatagcttgctagtaacagtggcctttattattttctgggtgaggagtaagaggagcaggctcctgca
cagtgactacatgaacatgactccccgccgccccgggcccacccgcaagcattaccagccctatgccccaccacgcgacttcgcagc
ctatcgctcctaagtcgacaggatccaagcctcgactgtgccttctagttgccagccatctgttgtttgcccctcccccgtgccttccttgacc
ctggaaggtgccactcccactgtcctttcctaataaaatgaggaaattgcatcgcattgtctgagtaggtgtcattctattctggggggtggg
gtggggcaggacagcaagggggaggattgggaagacaatagcaggcatgctggggatgcggtgggctgaattcactagctgcggct
gtggtccagctgaggtgaggggccttgaagctgggagtggggtttagggacgcgggtctctgggtgcatcctaagctctgagagcaaa
cctccctgcagggtcttgcttttaagtccaaagcctgagcccaccaaactctcctacttcttcctgttacaaattcctcttgtgcaataataatg
gcctgaaacgctgtaaaatatcctcatttcagccgcctcagttgcacttctcccctatgaggtaggaagaacagttgtttagaaacgaaga
aactgaggccccacagctaatgagtggaggaagagagacacttgtgtacaccacatgccttgtgttgtacttctctcaccgtgtaacctcc
tcatgtcctctctccccagtacggctctcttagctcagtagaaagaagacattacactcatattacaccccaatcctggctagagtctccgc
accctcctcccccagggtccccagtcgtcttgctgactagttgacacgggaaaaaagcacacgtgcggaccgagcggccgcaggaa
cccctagtgatggagttggccactccctctctgcgcgctcgctcgctcactgaggccgggcgaccaaaggtcgcccgacgcccgggctt
tgcccgggcggcctcagtgagcgagcgagcgcgcagctgcctgcagg
R3PGK (pAAV-HA gRNA3.4-PGK-CD7H1.BBZ-F2A-A40C2828)
SEQ ID NO: 169
cctgcaggcagctgcgcgctcgctcgctcactgaggccgcccgggcgtcgggcgacctttggtcgcccggcctcagtgagcgagcga
gcgcgcagagagggagtggccaactccatcactaggggttcctgcggccgcaacgcgtagatcctattaaataaaaggtcctgtctctc
aagaatcccctgccactcctcacacccaccctgggcccatattcatttccatttgagttgttcttattgagtcatccttcctgtggtagcggaac
tcactaaggggcccatctggacccgaggtattgtgatgataaattctgagcacctaccccatccccagaagggctcagaaataaaata
agagccaagtctagtcggtgtttcctgtcttgaaacacaatactgttggccctggaagaatgcacagaatctgtttgtaaggggatatgca
cagaagctgcaagggacaggaggtgcaggagctgcaggcctcccccacccagcctgctctgccttggggaaaaccgtgggtgtgtc
ctgcaggccatgcaggcctgggacatgcaagcccataaccgctgtggcctcttggttttacagatacgaacctaaactttcaaaacctgt
cagtgattgggttccgaatcctcctcctgaaagtggccaagctttaaggggttggggttgcgccttttccaaggcagccctgggtttgcgca
gggacgcggctgctctgggcgtggttccgggaaacgcagcggcgccgaccctgggtctcgcacattcttcacgtccgttcgcagcgtca
cccggatcttcgccgctacccttgtgggccccccggcgacgcttcctgctccgcccctaagtcgggaaggttccttgcggttcgcggcgtg
ccggacgtgacaaacggaagccgcacgtctcactagtaccctcgcagacggacagcgccagggagcaatggcagcgcgccgacc
gcgatgggctgtggccaatagcggctgctcagcagggcgcgccgagagcagcggccgggaaggggcggtgcgggaggcggggt
gtggggcggtagtgtgggccctgttcctgcccgcgcggtgttccgcattctgcaagcctccggagcgcacgtcggcagtcggctccctcg
ttgaccgaatcaccgacctctctccccagtctagaatggccttaccagtgaccgccttgctcctgccgctggccttgctgctccacgccgcc
aggccgcagcctgtgctgactcagtcaccgtcagcgtctgagacccccgggcagagggtcaccatctcttgttctgggagcacctccaa
caccggaaacaattatttttcttggtaccaacaagtcccaggaatggcccccaaactcctcatttataagaacaatctgcggccctcagg
ggtccctgaccgattctctggctccaagtctggcacctcagcctccctgtccatcagtggactccggtccgaggatgagggtgattattatt
gtgcaacatgggatgacaacttgactctggtgttcggcggagggaccaagctgaccgtcctaggtggtggtggttctggcggcggcggc
tccggaggtggtggatcccaggtgcagctgcaggagtcggggggaggcttggtacagcctggggggtccctgagactctcctgtgcag
cctctggattcacctttagcagctatgccatgagctgggtccgccaggctccagggaaggggctggagtgggtctcagctattagtggta
gtggtggtagcacatactacgcagactccgtgaagggccggttcaccatctccagagacaattccaagaacacgctgtatctgcaaatg
aacagcctgagagccgaggacacggccgtatattactgtgcgaaagggtacagtggctaccccgcagtgcttgactactggggcctg
ggaaccctggtcactgtctcctcaaccacgacgccagcgccgcgaccaccaacaccggcgcccaccatcgcgtcgcagcccctgtc
cctgcgcccagaggcgtgccggccagcggcggggggcgcagtgcacacgagggggctggacttcgcctgtgatatctacatctggg
cgcccttggccgggacttgtggggtccttctcctgtcactggttatcaccctttactgcaaacggggcagaaagaaactcctgtatatattca
aacaaccatttatgagaccagtacaaactactcaagaggaagatggctgtagctgccgatttccagaagaagaagaaggaggatgtg
aactgagagtgaagttcagcaggagcgcagacgcccccgcgtacaagcagggccagaaccagctctataacgagctcaatctagg
acgaagagaggagtacgacgttttggacaagagacgtggccgggaccctgagatggggggaaagccgagaaggaagaaccctc
aggaaggcctgtacaatgaactgcagaaagataagatggcggaggcctacagtgagattgggatgaaaggcgagcgccggaggg
gcaaggggcacgatggcctttaccagggtctcagtacagccaccaaggacacctacgacgcccttcacatgcaggccctgccccctc
gcgtgaaacagactttgaattttgaccttctcaagttggcgggagacgtggagtccaacccagggccgatggccttaccagtgaccgcct
tgctcctgccgctggccttgctgctccacgccgccaggccgcaggtccagctggtacaatctggtgctgaagtgaagaaaccaggagc
gtcggtgaaggtctcatgcacagcctcaggcttcaacatcaaggactactacgtgcactgggtcaagcaagctcccggacaggggctg
gagtggatggggagaattgacccggaagacggcgactcgaaatacgcgccgaagttccagggcaaggccaccatgacagccgat
accagtacatcgaccgtttacatggagctgtccagcctccgctcagaggataccgcggtatactactgtacgacctcctactacgtcgga
acgtacggttactggggccagggtaccctggtgacagtgtcctcggggggcggcgggtctggcgggggaggcagcgggggcggcg
ggtcggacatccagatgactcaatccccctcttccctgagtgcgtccgtgggtgacagggtgacgatcacttgtagcgcttcgtcctcggt
gtcgtatatgctgtggttccagcagaagcccggtaaggcccccaaactgctcatttattccaccagcaacctcgccagtggcgtacccag
tcgcttctccggtagtggctccggtaccgatttcacactgaccatctccagtctgcaacctgaagacttcgcaacgtactattgtcagcagc
gcacattctatccctatacgtttggggggggaccaaggtcgagatcaagcgcacagctagcattgaagttatgtatcctcctccttaccta
gacaatgagaagagcaatggaaccattatccatgtgaaagggaaacacctttgtccaagtcccctatttcccggaccttctaagccctttt
gggtgctggtggtggttggtggagtcctggcttgctatagcttgctagtaacagtggcctttattattttctgggtgaggagtaagaggagca
ggctcctgcacagtgactacatgaacatgactccccgccgccccgggcccacccgcaagcattaccagccctatgccccaccacgcg
acttcgcagcctatcgctcctaagtcgacaggatccaagcctcgactgtgccttctagttgccagccatctgttgtttgcccctcccccgtgc
cttccttgaccctggaaggtgccactcccactgtcctttcctaataaaatgaggaaattgcatcgcattgtctgagtaggtgtcattctattctg
gggggtggggggggcaggacagcaagggggaggattgggaagacaatagcaggcatgctggggatgcggtgggctgaattcact
agctgcggctgtggtccagctgaggtgaggggccttgaagctgggagtggggtttagggacgcgggtctctgggtgcatcctaagctct
gagagcaaacctccctgcagggtcttgcttttaagtccaaagcctgagcccaccaaactctcctacttcttcctgttacaaattcctcttgtgc
aataataatggcctgaaacgctgtaaaatatcctcatttcagccgcctcagttgcacttctcccctatgaggtaggaagaacagttgtttag
aaacgaagaaactgaggccccacagctaatgagtggaggaagagagacacttgtgtacaccacatgccttgtgttgtacttctctcacc
gtgtaacctcctcatgtcctctctccccagtacggctctcttagctcagtagaaagaagacattacactcatattacaccccaatcctggcta
gagtctccgcaccctcctcccccagggtccccagtcgtcttgctgactagttgacacgggaaaaaagcacacgtgcggaccgagcgg
ccgcaggaacccctagtgatggagttggccactccctctctgcgcgctcgctcgctcactgaggccgggcgaccaaaggtcgcccgac
gcccgggctttgcccgggcggcctcagtgagcgagcgagcgcgcagctgcctgcagg
CD7H1.BBZ-F2A-A40C2828, amino acid sequence
SEQ ID NO: 170
MALPVTALLLPLALLLHAARPQPVLTQSPSASETPGQRVTISCSGSTSNTGNNYFSWYQQVPG
MAPKLLIYKNNLRPSGVPDRFSGSKSGTSASLSISGLRSEDEGDYYCATWDDNLTLVFGGGTKLT
VLGGGGSGGGGSGGGGSQVQLQESGGGLVQPGGSLRLSCAASGFTFSSYAMSWVRQAPGK
GLEWVSAISGSGGSTYYADSVKGRFTISRDNSKNTLYLQMNSLRAEDTAVYYCAKGYSGYPAVL
DYWGLGTLVTVSSTTTPAPRPPTPAPTIASQPLSLRPEACRPAAGGAVHTRGLDFACDIYIWAPLA
GTCGVLLLSLVITLYCKRGRKKLLYIFKQPFMRPVQTTQEEDGCSCRFPEEEEGGCELRVKFSRS
ADAPAYKQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPRVKQTLNFDLLKLAGDVESNP
GPMALPVTALLLPLALLLHAARPQVQLVQSGAEVKKPGASVKVSCTASGFNIKDYYVHWVKQA
PGQGLEWMGRIDPEDGDSKYAPKFQGKATMTADTSTSTVYMELSSLRSEDTAVYYCTTSYYVGT
YGYWGQGTLVTVSSGGGGSGGGGGGGGSDIQMTQSPSSLSASVGDRVTITCSASSSVSYfW
FQQKPGKAPKLLIYSTSNLASGVPSRFSGSGSGTDFTLTISSLQPEDFATYYCQQRTFYPYTFGG
GTKVEIKRTASIEVMYPPPYLDNEKSNGTIIHVKGKHLCPSPLFPGPSKPFWVLVVVGGVLACYSL
LVTVAFIIFWVRSKRSRLLHSDYMNMTPRRPGPTRKHYQPYAPPRDFAAYRS
CD7H1.BBZ-F2A-A40C2828, nucleotide sequence
SEQ ID NO: 171
atggccttaccagtgaccgccttgctcctgccgctggccttgctgctccacgccgccaggccgcagcctgtgctgactcagtcaccgtca
gcgtctgagacccccgggcagagggtcaccatctcttgttctgggagcacctccaacaccggaaacaattatttttcttggtaccaacaa
gtcccaggaatggcccccaaactcctcatttataagaacaatctgcggccctcaggggtccctgaccgattctctggctccaagtctggc
acctcagcctccctgtccatcagtggactccggtccgaggatgagggtgattattattgtgcaacatgggatgacaacttgactctggtgtt
cggcggagggaccaagctgaccgtcctaggtggtggtggttctggcggcggcggctccggaggtggtggatcccaggtgcagctgca
ggagtcggggggaggcttggtacagcctggggggtccctgagactctcctgtgcagcctctggattcacctttagcagctatgccatgag
ctgggtccgccaggctccagggaaggggctggagtgggtctcagctattagtggtagtggtggtagcacatactacgcagactccgtga
agggccggttcaccatctccagagacaattccaagaacacgctgtatctgcaaatgaacagcctgagagccgaggacacggccgtat
attactgtgcgaaagggtacagtggctaccccgcagtgcttgactactggggcctgggaaccctggtcactgtctcctcaaccacgacg
ccagcgccgcgaccaccaacaccggcgcccaccatcgcgtcgcagcccctgtccctgcgcccagaggcgtgccggccagcggcg
gggggcgcagtgcacacgagggggctggacttcgcctgtgatatctacatctgggcgcccttggccgggacttgtggggtccttctcctgt
cactggttatcaccctttactgcaaacggggcagaaagaaactcctgtatatattcaaacaaccatttatgagaccagtacaaactactc
aagaggaagatggctgtagctgccgatttccagaagaagaagaaggaggatgtgaactgagagtgaagttcagcaggagcgcaga
cgcccccgcgtacaagcagggccagaaccagctctataacgagctcaatctaggacgaagagaggagtacgacgttttggacaag
agacgtggccgggaccctgagatggggggaaagccgagaaggaagaaccctcaggaaggcctgtacaatgaactgcagaaaga
taagatggcggaggcctacagtgagattgggatgaaaggcgagcgccggaggggcaaggggcacgatggcctttaccagggtctc
agtacagccaccaaggacacctacgacgcccttcacatgcaggccctgccccctcgcgtgaaacagactttgaattttgaccttctcaa
gttggcgggagacgtggagtccaacccagggccgatggccttaccagtgaccgccttgctcctgccgctggccttgctgctccacgccg
ccaggccgcaggtccagctggtacaatctggtgctgaagtgaagaaaccaggagcgtcggtgaaggtctcatgcacagcctcaggct
tcaacatcaaggactactacgtgcactgggtcaagcaagctcccggacaggggctggagtggatggggagaattgacccggaagac
ggcgactcgaaatacgcgccgaagttccagggcaaggccaccatgacagccgataccagtacatcgaccgtttacatggagctgtcc
agcctccgctcagaggataccgcggtatactactgtacgacctcctactacgtcggaacgtacggttactggggccagggtaccctggt
gacagtgtcctcggggggcggcgggtctggcgggggaggcagcgggggcggcgggtcggacatccagatgactcaatccccctctt
ccctgagtgcgtccgtgggtgacagggtgacgatcacttgtagcgcttcgtcctcggtgtcgtatatgctgtggttccagcagaagcccggt
aaggcccccaaactgctcatttattccaccagcaacctcgccagtggcgtacccagtcgcttctccggtagtggctccggtaccgatttca
cactgaccatctccagtctgcaacctgaagacttcgcaacgtactattgtcagcagcgcacattctatccctatacgtttggggggggac
caaggtcgagatcaagcgcacagctagcattgaagttatgtatcctcctccttacctagacaatgagaagagcaatggaaccattatcc
atgtgaaagggaaacacctttgtccaagtcccctatttcccggaccttctaagcccttttgggtgctggtggtggttggtggagtcctggcttg
ctatagcttgctagtaacagtggcctttattattttctgggtgaggagtaagaggagcaggctcctgcacagtgactacatgaacatgactc
cccgccgccccgggcccacccgcaagcattaccagccctatgccccaccacgcgacttcgcagcctatcgctcctaa
Homologous arm gRNA3.4-left (5′)
SEQ ID NO: 172
Gtcctgtctctcaagaatcccctgccactcctcacacccaccctgggcccatattcatttccatttgagttgttcttattgagtcatccttcctgtg
gtagcggaactcactaaggggcccatctggacccgaggtattgtgatgataaattctgagcacctaccccatccccagaagggctcag
aaataaaataagagccaagtctagtcggtgtttcctgtcttgaaacacaatactgttggccctggaagaatgcacagaatctgtttgtaag
gggatatgcacagaagctgcaagggacaggaggtgcaggagctgcaggcctcccccacccagcctgctctgccttggggaaaacc
gtgggtgtgtcctgcaggccatgcaggcctgggacatgcaagcccataaccgctgtggcctcttggttttacagatacgaacctaaacttt
caaaacctgtcagtgattgggttccgaatcctcctcctgaaagtggcc
Homologous arm gRNA3.4-right side (3′)
SEQ ID NO: 173
Ctgcggctgtggtccagctgaggtgaggggccttgaagctgggagtggggtttagggacgcgggtctctgggtgcatcctaagctctga
gagcaaacctccctgcagggtcttgcttttaagtccaaagcctgagcccaccaaactctcctacttcttcctgttacaaattcctcttgtgcaa
taataatggcctgaaacgctgtaaaatatcctcatttcagccgcctcagttgcacttctcccctatgaggtaggaagaacagttgtttagaa
acgaagaaactgaggccccacagctaatgagtggaggaagagagacacttgtgtacaccacatgccttgtgttgtacttctctcaccgt
gtaacctcctcatgtcctctctccccagtacggctctcttagctcagtagaaagaagacattacactcatattacaccccaatcctggctag
agtctccgcaccctcctcccccagggtccccagtcgtcttgctg
Homologous arm gRNA19.6-left (5′)
SEQ ID NO: 174
Agatcctattaaataaaagaataagcagtattattaagtagccctgcatttcaggtttccttgagtggcaggccaggcctggccgtgaacg
ttcactgaaatcatggcctcttggccaagattgatagcttgtgcctgtccctgagtcccagtccatcacgagcagctggtttctaagatgctat
ttcccgtataaagcatgagaccgtgacttgccagccccacagagccccgcccttgtccatcactggcatctggactccagcctgggttgg
ggcaaagagggaaatgagatcatgtcctaaccctgatcctcttgtcccacagatatccagaaccctgaccctgccgtgtaccagctgag
agactctaaatccagtgacaagtctgtctgcctat
Homologous arm gRNA19.6-right (3′)
SEQ ID NO: 175
tcaccgattttgaaaacaaatgtgtcacaaagtaaggattctgatgtgtatatcacagacaaaactgtgctagacatgaggtctatggactt
caagagcaacagtgctgtggcctggagcaacaaatctgactttgcatgtgcaaacgccttcaacaacagcattattccagaagacacct
tcttccccagcccaggtaagggcagctttggtgccttcgcaggctgtttccttgcttcaggaatggccaggttctgcccagagctctggtca
atgatgtctaaaactcctctgattggtggtctcggccttatccattgccaccaaaaccctctttttactaagaaacagtgagccttgttctggca
gtccagagaatgacacgggaaaaaagca
PGK promoter
SEQ ID NO: 176
Ggggttggggttgcgccttttccaaggcagccctgggtttgcgcagggacgcggctgctctgggcgtggttccgggaaacgcagcggc
gccgaccctgggtctcgcacattcttcacgtccgttcgcagcgtcacccggatcttcgccgctacccttgtgggccccccggcgacgcttc
ctgctccgcccctaagtcgggaaggttccttgcggttcgcggcgtgccggacgtgacaaacggaagccgcacgtctcactagtaccctc
gcagacggacagcgccagggagcaatggcagcgcgccgaccgcgatgggctgtggccaatagcggctgctcagcagggcgcgc
cgagagcagcggccgggaaggggcggtgcgggaggcggggtgtggggcggtagtgtgggccctgttcctgcccgcgcggtgttccg
cattctgcaagcctccggagcgcacgtcggcagtcggctccctcgttgaccgaatcaccgacctctctccccag
Linker 1
SEQ ID NO: 177
GGGGSGGGGSGGGGS
Linker 2
SEQ ID NO: 178
GGGSGGGGSGGGGS
F2A
SEQ ID NO: 179
VKQTLNFDLLKLAGDVESNPGP

1. Modified T cells, wherein the expression of endogenous TRAC, B2M and CD7 genes in said T cells is suppressed, and wherein said T cells express a chimeric antigen receptor targeting CD7, said chimeric antigen receptor comprising: a binding domain that specifically binds to CD7.

2. (canceled)

3. The modified T cells of claim 1, wherein the light chain variable region and heavy chain variable region of the binding domain that specifically binds to CD7 comprise LCDR1, LCDR2, LCDR3, HCDR1, HCDR2 and HCDR3 selected from the group consisting of:
(a) an LCDR1 having the amino acid sequence shown in SEQ ID NO:113, an LCDR2 having the amino acid sequence shown in SEQ ID NO:126, an LCDR3 having the amino acid sequence shown in SEQ ID NO:138, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(b) an LCDR1 having the amino acid sequence shown in SEQ ID NO:114, an LCDR2 having the amino acid sequence shown in SEQ ID NO:127, an LCDR3 having the amino acid sequence shown in SEQ ID NO:139, an HCDR1 having the amino acid sequence shown in SEQ ID NO:88, an HCDR2 having the amino acid sequence shown in SEQ ID NO:96, and an HCDR3 having the amino acid sequence shown in SEQ ID NO:104;
(c) an LCDR1 having the amino acid sequence shown in SEQ ID NO:115, an LCDR2 having the amino acid sequence shown in SEQ ID NO:128, an LCDR3 having the amino acid sequence shown in SEQ ID NO:140, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:105;
(d) an LCDR1 having the amino acid sequence shown in SEQ ID NO:116, an LCDR2 having the amino acid sequence shown in SEQ ID NO:129, an LCDR3 having the amino acid sequence shown in SEQ ID NO:141, an HCDR1 having the amino acid sequence shown in SEQ ID NO:89, an HCDR2 having the amino acid sequence shown in SEQ ID NO:97, and an HCDR3 having the amino acid sequence shown in SEQ ID NO:106;
(e) an LCDR1 having the amino acid sequence shown in SEQ ID NO:117, an LCDR2 having the amino acid sequence shown in SEQ ID NO:130, an LCDR3 having the amino acid sequence shown in SEQ ID NO:142, an HCDR1 having the amino acid sequence shown in SEQ ID NO:90, an HCDR2 having the amino acid sequence shown in SEQ ID NO:98, and an HCDR3 having the amino acid sequence shown in SEQ ID NO:107;
(f) an LCDR1 having the amino acid sequence shown in SEQ ID NO:118, an LCDR2 having the amino acid sequence shown in SEQ ID NO:131, an LCDR3 having the amino acid sequence shown in SEQ ID NO:143, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(g) an LCDR1 having the amino acid sequence shown in SEQ ID NO:119, an LCDR2 having the amino acid sequence shown in SEQ ID NO:132, an LCDR3 having the amino acid sequence shown in SEQ ID NO:144, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(h) an LCDR1 having the amino acid sequence shown in SEQ ID NO:120, an LCDR2 having the amino acid sequence shown in SEQ ID NO:133, an LCDR3 having the amino acid sequence shown in SEQ ID NO:145, an HCDR1 having the amino acid sequence shown in SEQ ID NO:91, an HCDR2 having the amino acid sequence shown in SEQ ID NO:99, and an HCDR3 having the amino acid sequence shown in SEQ ID NO:108;
(i) an LCDR1 having the amino acid sequence shown in SEQ ID NO:121, an LCDR2 having the amino acid sequence shown in SEQ ID NO:134, an LCDR3 having the amino acid sequence shown in SEQ ID NO:146, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(j) an LCDR1 having the amino acid sequence shown in SEQ ID NO:122, an LCDR2 having the amino acid sequence shown in SEQ ID NO:135, an LCDR3 having the amino acid sequence shown in SEQ ID NO:147, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:109;
(k) an LCDR1 having the amino acid sequence shown in SEQ ID NO:123, an LCDR2 having the amino acid sequence shown in SEQ ID NO:136, an LCDR3 having the amino acid sequence shown in SEQ ID NO:148, an HCDR1 having the amino acid sequence shown in SEQ ID NO:92, an HCDR2 having the amino acid sequence shown in SEQ ID NO:100, and an HCDR3 having the amino acid sequence shown in SEQ ID NO:110;
(l) an LCDR1 having the amino acid sequence shown in SEQ ID NO:124, an LCDR2 having the amino acid sequence shown in SEQ ID NO:137, an LCDR3 having the amino acid sequence shown in SEQ ID NO:149, an HCDR1 having the amino acid sequence shown in SEQ ID NO:93, an HCDR2 having the amino acid sequence shown in SEQ ID NO:101, and an HCDR3 having the amino acid sequence shown in SEQ ID NO:111; and
(m) an LCDR1 having the amino acid sequence shown in SEQ ID NO:125, an LCDR2 having the amino acid sequence shown in SEQ ID NO:137, an LCDR3 having the amino acid sequence shown in SEQ ID NO:150, an HCDR1 having the amino acid sequence shown in SEQ ID NO:94, an HCDR2 having the amino acid sequence shown in SEQ ID NO:102, and an HCDR3 having the amino acid sequence shown in SEQ ID NO:112.

4. The modified T cells of claim 1, wherein the binding domain that specifically binds to CD7 comprises a light chain variable region and a heavy chain variable region selected from the group consisting of:
(a) a light chain variable region comprising the amino acid sequence shown in SEQ ID NO:35 and a heavy chain variable region comprising the amino acid sequence shown in SEQ ID NO:36;
(b) a light chain variable region comprising the amino acid sequence shown in SEQ ID NO:40 and a heavy chain variable region comprising the amino acid sequence shown in SEQ ID NO:41;
(c) a light chain variable region comprising the amino acid sequence shown in SEQ ID NO:44 and a heavy chain variable region comprising the amino acid sequence shown in SEQ ID NO:45;
(d) a light chain variable region comprising the amino acid sequence shown in SEQ ID NO:48 and a heavy chain variable region comprising the amino acid sequence shown in SEQ ID NO:49;
(e) a light chain variable region comprising the amino acid sequence shown in SEQ ID NO:52 and a heavy chain variable region comprising the amino acid sequence shown in SEQ ID NO:53;
(f) a light chain variable region comprising the amino acid sequence shown in SEQ ID NO:56 and a heavy chain variable region comprising the amino acid sequence shown in SEQ ID NO:57;
(g) a light chain variable region comprising the amino acid sequence shown in SEQ ID NO:60 and a heavy chain variable region comprising the amino acid sequence shown in SEQ ID NO:61;
(h) a light chain variable region comprising the amino acid sequence shown in SEQ ID NO:64 and a heavy chain variable region comprising the amino acid sequence shown in SEQ ID NO:65;
(i) a light chain variable region comprising the amino acid sequence shown in SEQ ID NO:68 and a heavy chain variable region comprising the amino acid sequence shown in SEQ ID NO:69;
(j) a light chain variable region comprising the amino acid sequence shown in SEQ ID NO:72 and a heavy chain variable region comprising the amino acid sequence shown in SEQ ID NO:73;
(k) a light chain variable region comprising the amino acid sequence shown in SEQ ID NO:76 and a heavy chain variable region comprising the amino acid sequence shown in SEQ ID NO:77;
(l) a light chain variable region comprising the amino acid sequence shown in SEQ ID NO:80 and a heavy chain variable region comprising the amino acid sequence shown in SEQ ID NO:81; and
(m) a light chain variable region comprising the amino acid sequence shown in SEQ ID NO:84 and a heavy chain variable region comprising the amino acid sequence shown in SEQ ID NO:85.

5.-9. (canceled)

10. The modified T cells of claim 1, wherein the chimeric antigen receptor targeting CD7 comprises an amino acid sequence selected from SEQ ID NOs: 37, 42, 46, 50, 54, 58, 62, 66, 70, 74, 78, 82, 86.

11.-12. (canceled)

13. The modified T cells of claim 1, wherein the nucleic acid sequence encoding the chimeric antigen receptor is inserted into the TRAC locus of the T cells.

14. The modified T cells of claim 1, wherein the nucleic acid sequence encoding the chimeric antigen receptor is operably linked to a PGK promoter or an endogenous TRAC promoter of the T cells.

15. The modified T cells of claim 1, wherein the T cells further express a fusion protein of a lymphocyte-antigen presenting cell co-stimulator, wherein the fusion protein comprises a first domain that activates antigen presenting cells (APCs) and a second domain that activates immune effector cells, wherein
The first domain comprises: (i) a ligand that binds to an activating receptor of the APC, or a receptor-binding fragment thereof, or (ii) an antibody that binds to an activating receptor of the APC, or an antigen-binding fragment thereof; and
The second domain comprises: (i) a co-stimulatory receptor of the immune effector cell, or a functional fragment thereof, (ii) a co-stimulatory ligand of the immune effector cell, or a receptor binding fragment thereof, or (iii) an antibody that binds to the co-stimulatory receptor of the immune effector cell, or an antigen-binding fragment thereof.

16. (canceled)

17. The modified T cells of claim 15, wherein the first domain is an anti-CD40 antibody or an antigen-binding fragment thereof.

18.-27. (canceled)

28. The modified T cells of claim 1, wherein endogenous TRAC, B2M and CD7 genes are knocked out in the T cells.

29. A method for producing modified T cells, the method comprising:
(a) suppressing the expression of endogenous TRAC, B2M and CD7 genes in the T cells; and
(b) introducing a polynucleotide sequence comprising a nucleic acid sequence encoding a chimeric antigen receptor targeting CD7 into the T cell, so that the T cell expresses the chimeric antigen receptor, wherein the chimeric antigen receptor targeting CD7 comprises a binding domain that specifically binds to CD7.

30. (canceled)

31. The method of claim 29, wherein the light chain variable region and heavy chain variable region of the binding domain that specifically binds to CD7 comprise LCDR1, LCDR2, LCDR3, HCDR1, HCDR2 and HCDR3 selected from the group consisting of:
(a) an LCDR1 having the amino acid sequence shown in SEQ ID NO:113, an LCDR2 having the amino acid sequence shown in SEQ ID NO:126, an LCDR3 having the amino acid sequence shown in SEQ ID NO:138, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(b) an LCDR1 having the amino acid sequence shown in SEQ ID NO:114, an LCDR2 having the amino acid sequence shown in SEQ ID NO:127, an LCDR3 having the amino acid sequence shown in SEQ ID NO:139, an HCDR1 having the amino acid sequence shown in SEQ ID NO:88, an HCDR2 having the amino acid sequence shown in SEQ ID NO:96, and an HCDR3 having the amino acid sequence shown in SEQ ID NO:104;
(c) an LCDR1 having the amino acid sequence shown in SEQ ID NO:115, an LCDR2 having the amino acid sequence shown in SEQ ID NO:128, an LCDR3 having the amino acid sequence shown in SEQ ID NO:140, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:105;
(d) an LCDR1 having the amino acid sequence shown in SEQ ID NO:116, an LCDR2 having the amino acid sequence shown in SEQ ID NO:129, an LCDR3 having the amino acid sequence shown in SEQ ID NO:141, an HCDR1 having the amino acid sequence shown in SEQ ID NO:89, an HCDR2 having the amino acid sequence shown in SEQ ID NO:97, and an HCDR3 having the amino acid sequence shown in SEQ ID NO:106;
(e) an LCDR1 having the amino acid sequence shown in SEQ ID NO:117, an LCDR2 having the amino acid sequence shown in SEQ ID NO:130, an LCDR3 having the amino acid sequence shown in SEQ ID NO:142, an HCDR1 having the amino acid sequence shown in SEQ ID NO:90, an HCDR2 having the amino acid sequence shown in SEQ ID NO:98, and an HCDR3 having the amino acid sequence shown in SEQ ID NO:107;
(f) an LCDR1 having the amino acid sequence shown in SEQ ID NO:118, an LCDR2 having the amino acid sequence shown in SEQ ID NO:131, an LCDR3 having the amino acid sequence shown in SEQ ID NO:143, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(g) an LCDR1 having the amino acid sequence shown in SEQ ID NO:119, an LCDR2 having the amino acid sequence shown in SEQ ID NO:132, an LCDR3 having the amino acid sequence shown in SEQ ID NO:144, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(h) an LCDR1 having the amino acid sequence shown in SEQ ID NO:120, an LCDR2 having the amino acid sequence shown in SEQ ID NO:133, an LCDR3 having the amino acid sequence shown in SEQ ID NO:145, an HCDR1 having the amino acid sequence shown in SEQ ID NO:91, an HCDR2 having the amino acid sequence shown in SEQ ID NO:99, and an HCDR3 having the amino acid sequence shown in SEQ ID NO:108;
(i) an LCDR1 having the amino acid sequence shown in SEQ ID NO:121, an LCDR2 having the amino acid sequence shown in SEQ ID NO:134, an LCDR3 having the amino acid sequence shown in SEQ ID NO:146, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:103;
(j) an LCDR1 having the amino acid sequence shown in SEQ ID NO:122, an LCDR2 having the amino acid sequence shown in SEQ ID NO:135, an LCDR3 having the amino acid sequence shown in SEQ ID NO:147, an HCDR1 having the amino acid sequence shown in SEQ ID NO:87, an HCDR2 having the amino acid sequence shown in SEQ ID NO:95, and an HCDR3 having the amino acid sequence shown in SEQ ID NO:109;
(k) an LCDR1 having the amino acid sequence shown in SEQ ID NO:123, an LCDR2 having the amino acid sequence shown in SEQ ID NO:136, an LCDR3 having the amino acid sequence shown in SEQ ID NO:148, an HCDR1 having the amino acid sequence shown in SEQ ID NO:92, an HCDR2 having the amino acid sequence shown in SEQ ID NO:100, and an HCDR3 having the amino acid sequence shown in SEQ ID NO:110;
(l) an LCDR1 having the amino acid sequence shown in SEQ ID NO:124, an LCDR2 having the amino acid sequence shown in SEQ ID NO:137, an LCDR3 having the amino acid sequence shown in SEQ ID NO:149, an HCDR1 having the amino acid sequence shown in SEQ ID NO:93, an HCDR2 having the amino acid sequence shown in SEQ ID NO:101, and an HCDR3 having the amino acid sequence shown in SEQ ID NO:111; and
(m) an LCDR1 having the amino acid sequence shown in SEQ ID NO:125, an LCDR2 having the amino acid sequence shown in SEQ ID NO:137, an LCDR3 having the amino acid sequence shown in SEQ ID NO:150, an HCDR1 having the amino acid sequence shown in SEQ ID NO:94, an HCDR2 having the amino acid sequence shown in SEQ ID NO:102, and an HCDR3 having the amino acid sequence shown in SEQ ID NO:112.

32. The method of claim 29, wherein the binding domain that specifically binds to CD7 comprises a light chain variable region and a heavy chain variable region selected from the group consisting of:
(a) a light chain variable region comprising the amino acid sequence shown in SEQ ID NO:35 and a heavy chain variable region comprising the amino acid sequence shown in SEQ ID NO:36;
(b) a light chain variable region comprising the amino acid sequence shown in SEQ ID NO:40 and a heavy chain variable region comprising the amino acid sequence shown in SEQ ID NO:41;
(c) a light chain variable region comprising the amino acid sequence shown in SEQ ID NO:44 and a heavy chain variable region comprising the amino acid sequence shown in SEQ ID NO:45;
(d) a light chain variable region comprising the amino acid sequence shown in SEQ ID NO:48 and a heavy chain variable region comprising the amino acid sequence shown in SEQ ID NO:49;
(e) a light chain variable region comprising the amino acid sequence shown in SEQ ID NO:52 and a heavy chain variable region comprising the amino acid sequence shown in SEQ ID NO:53;
(f) a light chain variable region comprising the amino acid sequence shown in SEQ ID NO:56 and a heavy chain variable region comprising the amino acid sequence shown in SEQ ID NO:57;
(g) a light chain variable region comprising the amino acid sequence shown in SEQ ID NO:60 and a heavy chain variable region comprising the amino acid sequence shown in SEQ ID NO:61;
(h) a light chain variable region comprising the amino acid sequence shown in SEQ ID NO:64 and a heavy chain variable region comprising the amino acid sequence shown in SEQ ID NO:65;
(i) a light chain variable region comprising the amino acid sequence shown in SEQ ID NO:68 and a heavy chain variable region comprising the amino acid sequence shown in SEQ ID NO:69;
(j) a light chain variable region comprising the amino acid sequence shown in SEQ ID NO:72 and a heavy chain variable region comprising the amino acid sequence shown in SEQ ID NO:73;
(k) a light chain variable region comprising the amino acid sequence shown in SEQ ID NO:76 and a heavy chain variable region comprising the amino acid sequence shown in SEQ ID NO:77;
(l) a light chain variable region comprising the amino acid sequence shown in SEQ ID NO:80 and a heavy chain variable region comprising the amino acid sequence shown in SEQ ID NO:81; and
(m) a light chain variable region comprising the amino acid sequence shown in SEQ ID NO:84 and a heavy chain variable region comprising the amino acid sequence shown in SEQ ID NO:85.

33.-37. (canceled)

38. The method of claim 29, wherein the chimeric antigen receptor targeting CD7 comprises an amino acid sequence selected from SEQ ID NO:37, 42, 46, 50, 54, 58, 62, 66, 70, 74, 78, 82, 86.

39.-40. (canceled)

41. The method of claim 29, wherein step (a) comprises knocking out endogenous TRAC, B2M and CD7 genes in the T cells.

42.-46. (canceled)

47. The method of claim 29, wherein the polynucleotide sequence comprises homology arms flanking the nucleic acid sequence encoding the chimeric antigen receptor and homologous to the upstream and downstream sequences of the TRAC gene cleavage site, respectively, so that the nucleic acid sequence encoding the chimeric antigen receptor is inserted into the TRAC gene locus by homologous recombination.

48. The method of claim 29, wherein the nucleic acid sequence encoding the chimeric antigen receptor is inserted into the TRAC locus at a position operably linked to the TRAC endogenous promoter;
or the polynucleotide sequence comprises a PGK promoter operably linked to a nucleic acid sequence encoding a chimeric antigen receptor.

49.-51. (canceled)

52. The method of claim 29, further comprising:
(c) introducing a polynucleotide sequence comprising a nucleic acid sequence encoding a lymphocyte-antigen presenting cell co-stimulator fusion protein into the T cell,
The lymphocyte-antigen presenting cell co-stimulatory fusion protein comprises a first domain for activating antigen presenting cells (APCs) and a second domain for activating immune effector cells, wherein
The first domain comprises: (i) a ligand that binds to an activating receptor of the APC, or a receptor binding fragment thereof, or (ii) an antibody that binds to an activating receptor of the APC, or an antigen-binding fragment thereof; and
The second domain comprises: (i) a co-stimulatory receptor of the immune effector cell, or a functional fragment thereof, (ii) a co-stimulatory ligand of the immune effector cell, or a receptor binding fragment thereof, or (iii) an antibody that binds to the co-stimulatory receptor of the immune effector cell, or an antigen-binding-fragment thereof.

53. (canceled)

54. The method of claim 52, wherein the first domain is an anti-CD40 antibody or an antigen-binding fragment thereof.

55.-64. (canceled)

65. A pharmaceutical composition comprising the modified T cells of claim 1 and a pharmaceutically acceptable carrier.

66. A method for treating a disease associated with CD7 expression, comprising administering a therapeutically effective amount of the modified T cells of claim 1 to a subject in need thereof.

67. (canceled)
